SEQUENCE LISTING (4 sequences)

SEQ ID NO: 1 - protein, 447 aa, synthetic construct
QVQLVQSGAEVKKPGASVKVSCKASGYSFTGYFMNWVRQAPGQGLEWMGRIHPYDGDTFYAQKFQGRVTMTVDKSISTAYMELSRLRSDDTVVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 218 aa, synthetic construct
EIVLTQSPATLSLSPGERATLSCRASQSVSFAGTSLLHWYQQKPGQAPRLLIYRASNLETGIPARFSGSGSKTDFTLTISSLEPEDFAVYYCQQSREYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 472 aa, synthetic construct
MDPKGSLSWRILLFLSLAFELSYGQVQLVQSGAEVKKPGASVKVSCKASDYTFTYYGINWVRQAPGQGLEWMGWIDTTTGEPNYAQKLQGRVTFTLDTSASTAYMELRSLRSDDTAVYYCARRGPYNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 239 aa, synthetic construct
METDTLLLWVLLLWVPGSTGDIVMTQTPLSLPVTPGEPASISCRSSKSLLHSNGNTYLYWYLQKPGQSPQLLIYRMSNRASGVPDRFSGSGSGTDFTLKISRVEAEDVGVYYCLQHLEYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0345812 A1
(43) Pub. Date: Nov. 5, 2026

(54) ANTIBODY-DRUG CONJUGATE COMPOUNDS, AND METHODS OF USE AND TREATMENT

(71) Applicants: IKSUDA THERAPEUTICS LIMITED, Newcastle Upon Tyne, Tyne & Wear (GB); GEORG-AUGUST-UNIVERSITÄT GÖTTINGEN STIFTUNG ÖFFENTLICHEN RECHTS, Göttingen (DE)

(72) Inventors: Justyna Helena MYSLIWY, Newcastle upon Tyne (GB); Patrick Louis KERIGAN HIGGS, Newcastle upon Tyne (GB); Daniel John WILLIAMSON, Olesa de Montserrat (ES); Lutz F. TIETZE, Göttingen (DE); Mehrnoush KANGANI, Göttingen (DE)

(73) Assignees: IKSUDA THERAPEUTICS LIMITED, Newcastle Upon Tyne, Tyne & Wear (GB); GEORG-AUGUST-UNIVERSITÄT GÖTTINGEN STIFTUNG ÖFFENTLICHEN RECHTS, Göttingen (DE)

(21) Appl. No.: 19/476,651

(22) PCT Filed: Apr. 29, 2024
(86) PCT No.: PCT/GB2024/051130

§ 371 Date: Oct. 17, 2025

(30) Foreign Application Priority Data
Apr. 28, 2023 (GB) 2306325.8

(51) Int. Cl. A61K 47/68

(52) U.S. Cl. CPC A61K 47/6889; A61K 47/6803; A61K 47/6849; A61K 47/6851; A61K 47/6855; A61K 47/6869

(57) ABSTRACT

The present invention relates generally to antibodies conjugated to cyclopropylbenzoindole (CBI) dimer drug moieties to form antibody-drug conjugates and to compositions comprising the antibody-drug conjugates. Methods of making the antibody-drug conjugates are also provided. Methods of using the antibody-drug conjugates and compositions, including for the treatment of cancer, are further provided.

### FIELD OF THE INVENTION

The present invention relates generally to antibodies conjugated to cyclopropylbenzoindole (CBI) dimer drug moieties to form antibody-drug conjugates and to compositions comprising the antibody-drug conjugates. Methods of making the antibody-drug conjugates are also provided. Methods of using the antibody-drug conjugates and compositions, including for the treatment of cancer, are further provided.

### BACKGROUND OF THE INVENTION

Antibody-drug conjugates (ADCs) are a class of therapeutics that exploit the specificity of monoclonal antibodies (mAbs) to enable targeted delivery of a cytotoxic payload. Typically, the payload by itself is highly toxic and so unsuitable for direct administration. ADCs function by using the antibody to target a specific antigen associated with cancerous cells and then releasing the drug payload under selected conditions to induce cell death. This enables the targeted delivery of the highly potent drug directly into the tumour, thereby reducing systemic exposure and toxicity to normal tissues. Accordingly, ADCs have significant potential to improve the treatment and survival of patients suffering from diseases, such as cancer.

Three key elements typically define an ADC: the antibody, the cytotoxic drug (also called payload) and the linker connecting the drug to the antibody. The role of the linker is to maintain a stable linkage between the antibody and the drug during circulation while also providing a mechanism that allows efficient drug release to the targeted cancer cells.

Despite the relatively straightforward molecular platform of ADCs, their clinical development has been hampered by multiple factors, including a narrow therapeutic index (a ratio that compares toxic dose to efficacious dose), appropriate antibody selection, linker design and stability, and the internalisation rate of the payloads. As such, the successful generation of an efficacious and highly stable ADC requires optimisation of all these factors. A low or narrow therapeutic index, in particular, continues to be a problem and accounts for the discontinuance of many ADCs in clinical development. A narrow therapeutic window limits the dose that can be achieved, often resulting in toxic effects occurring before an ADC reaches its maximally efficacious dose.

Accordingly, there continues to be a need to identify and develop ADCs for targeted treatments and medicaments that exhibit a combination of high therapeutic efficacy and high in-vivo stability. One approach for improvement in this context may be the use of prodrug forms of highly potent cytotoxic drugs. A prodrug derivative possesses a functional group that must be cleaved in addition to the linker before the active cytotoxic drug is released, thereby mitigating against side effects caused by premature decomposition of the linker. Thus, the use of prodrugs in combination with the targeting capabilities of monoclonal antibodies in ADCs may offer more effective treatments of cancer while further reducing side effects, namely, on healthy cells.

Cyclopropylbenzoindole (CBI)-based dimers are a class of DNA minor groove alkylators that are reported to be highly cytotoxic (see Tietze et al., Angew. Chem. Int. Ed. Engl. 2010, 49, 7336-7339) and have been developed into prodrugs for cancer therapy. For instance, halogen-containing seco derivatives of CBI are generally believed to require a ring closure to their cyclopropyl containing spiro analogues to become cytotoxic. Thus, by trapping the CBI unit in its seco form through the protection of the hydroxyl group by a prodrug functional group, the drug can be substantially inactivated until the prodrug function group is removed such that ring closure to the spiro form can occur. In one example, the attachment of phosphate groups to the hydroxyl group of CBI has been proposed. However, such prodrugs are still associated with safety concerns due to poor stability in blood after administration.

The present invention aims to alleviate at least some of the problems with the prior art.

### SUMMARY OF INVENTION

The present invention is directed to antibody-drug conjugates comprising CBI-dimers, and to methods for using the same to treat cancer. Thus, the present invention relates to compounds and pharmaceutical compositions containing them, to their preparation, and to uses of the compounds as anti-cancer agents.

Advantageously, the ADCs of the present invention exhibit high efficacy as well as improved safety profiles.

In a first aspect, there is provided an antibody conjugate or a pharmaceutically acceptable salt or solvate thereof having the formula:

Ab-(L-D)n

wherein:
Ab is an antibody or antigen-binding fragment thereof;
D is a prodrug;
L is a linker covalently connecting Ab to D;
n is an integer from 1 to 20;
   wherein:
D is represented by formula I:

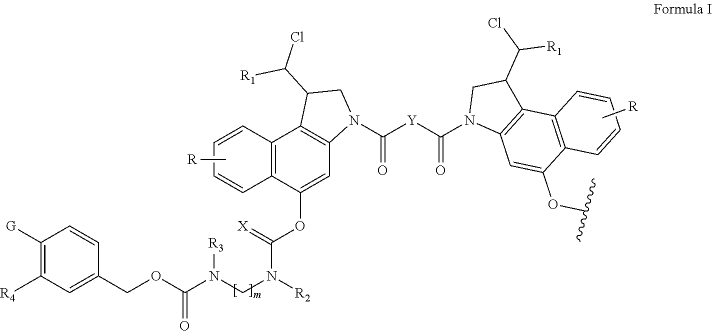

wherein:
the wavy line indicates the covalent attachment to L;
R is H or an optionally substituted C₁-C₄ alkyl group, an optionally substituted C₁-C₄ alkoxy group, an optionally substituted aryl group, an optionally substituted heteroaryl group, an optionally substituted C₁-C₄ alkyl carboxy C₁-C₄ alkyl group, F, Cl, Br, or I, CN, an optionally substituted C₁-C₄ alkylsulfonyl group, an optionally substituted arylsulfonyl group, or an NR_{Z} group, where NR_{Z} is selected from H, optionally substituted C₁-C₄ alkyl group or optionally substituted C₁-C₄ acyl;
R₁ is H, a C₁-C₄ alkyl group, or a C₁-C₄ alkoxy group;
Y is selected from an optionally substituted C₁-C₁₀ alkyl group, or a group having the formula:

ₒ[C(R_{A})₂]—X₃—[C(R_{A})₂]ₚ
where o and p are independently from one another selected from an integer of 1 to 20, whereby o and p may be the same integer or a different integer, X₃ is i) N, S or O, or ii) an aryl group or a heteroaryl group, wherein [C(R_{A})₂]ₒ and [C(R_{A})₂]ₚ are present in the meta position of said aryl group or said heteroaryl group, and each R_{A} is independently from one another selected from H or an optionally substituted C₁-C₄ alkyl group or an optionally substituted C₁-C₄ acyl group;
X is O or S;
m is an integer from 1 to 20;
R₂ and R₃ are independently selected from H, C₁-C₂₀ alkyl, and —C₁-C₈ heteroalkyl;
R₄ is selected from H or an electron-withdrawing group, and
G is a sugar moiety selected from β-D-galactoside, β-D-glucuronide, β-D-glucoside, α-D-mannoside, or fucose.

Accordingly, ADCs of the present invention comprise a CBI dimer prodrug whereby a first CBI unit of the CBI dimer incorporates a prodrug functional group and a second unit of the CBI dimer is attached to the antibody vialinker L. Thus, ADCs of the present invention incorporate a two-step activation mechanism, whereby the prodrug functional group must be cleaved in addition to the linker before the active cytotoxic drug is released. Advantageously, the CBI dimer prodrugs according to the present invention are believed to be more stable in circulation than the active form of the drug, thereby reducing the occurrence of side effects caused by the premature decomposition of the linker in normal cells.

The prodrug functional group comprises a sugar moiety represented by G that is coupled to a self-immolative linker via a glycosidic bond. The self-immolative linker provides a stable linkage between the sugar moiety and the first CBI unit and collapses upon the cleavage of glycosidic bond to liberate a free phenol at the first CBI unit. For example, the self-immolative linker may comprise para-hydroxybenzyloxycarbonyl or derivative thereof and a bisamine cyclization spacer. Advantageously, the inclusion of a self-immolative linker may improve the efficacy of the ADC compared to direct attachment of sugar moiety to the first CBI unit.

The prodrug functional group may be cleaved by enzymes that are overexpressed in tumours, resulting in higher tumour selectivity of the active form of the drug. An example of this is the β-D-glucuronidase, which has been detected in increased concentrations in necrotic areas of tumor tissue. Thus, in one example, G may be β-D-glucuronide. In another example, G may be β-D-galactoside.

Optionally, R₂ and R₃ may be independently selected from —C₁-C₂₀ alkyl and —(CH₂CH₂O)ᵣH, where r is an integer from 1 to 4. The hydrophobicity of some cleavable linker systems can lead to the aggregation of drug conjugates, particularly with strongly hydrophobic drugs. Advantageously, the incorporation of one or more hydrophilic substituents in these positions may reduce linker-drug hydrophobicity, which may decrease aggregation of the ADC.

The Linker (L) (sometimes referred to as “linker” herein) is a bifunctional compound which can be used to link the drug and the antibody. Various examples have been described in the art for linking the antibody moiety to the payload. These linker systems can generally be categorised as either cleavable or non-cleavable. For cleavable ADC linker systems, the release mechanism is typically enzymatically driven, although chemically labile cleavable systems are also known. In non-cleavable ADC linker systems, drug release is effected by degradation within the cell after internalisation of the ADC.

Optionally, L has the formula:

L^{A}-L^{B}-L^{C}-L^{D}

### Wherein:

L^{A} is connecting group or bond linking Ab to L^{B};
L^{B} is an enzyme cleavable linker;
L^{C} is a self-immolative spacer or absent; and
L^{D} is spacer group covalently bound to D.

The connecting group L^{A} may comprise a reactive site e.g., an electrophilic group, that is reactive to a nucleophilic group present on the antibody. Useful nucleophilic groups on an antibody include but are not limited to, sulfhydryl, hydroxyl and amino groups. The heteroatom of the nucleophilic group of an antibody is reactive to an electrophilic group on the connecting group and forms a covalent bond thereto. Useful electrophilic groups include, but are not limited to, vinylpyridine, maleimide and haloacetamide groups. Alternatively, connecting group L^{A} may comprise a reactive site which has a nucleophilic group that is reactive to an electrophilic group present on the antibody. Useful electrophilic groups on an antibody include, but are not limited to, aldehyde and ketone carbonyl groups. The heteroatom of a nucleophilic group of a linker unit can react with an electrophilic group on an antibody and form a covalent bond to the antibody. Useful nucleophilic groups on a linker unit include, but are not limited to, hydrazide, oxime, amino, hydrazine, thiosemicarbazone, hydrazine carboxylate, and arylhydrazide. Alternatively, the antibody may be modified to include an azide group (—Na) and the connecting group may incorporate an azide reactive moiety, such as a cyclooctyne group, for production of the antibody-drug conjugate according to the present invention. In this way, the linker can be bound to the antibody through a click-reaction between the azide group in the antibody and the cyclooctyne group in the linker.

Optionally, L_{B} may be selected from a β-D-glucuronide linker or a β-D-galactoside linker and L^{C} is absent. As used herein, a β-D-glucuronide linker comprises the formula:

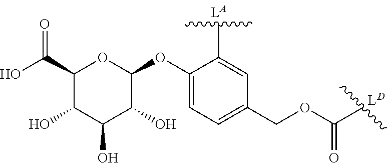 [SMILES: *C(=O)OCc1ccc(O[C@@H]2O[C@H](C(=O)O)[C@@H](O)[C@H](O)[C@H]2O)c(*)c1]

As used herein, a β-D-galactoside linker comprises the formula:

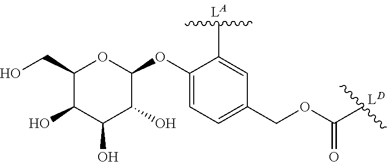 [SMILES: *C(=O)OCc1ccc(O[C@@H]2O[C@H](CO)[C@H](O)[C@H](O)[C@H]2O)c(*)c1]

Alternatively, L^{B} may be amino acids or a peptide, such as a dipeptide. For example, the dipeptide linker may be selected from -Valine-Citrulline- (-Val-Cit-), -Valine-Alanine- (-Val-Ala-), -Valine-Lysine- (-Val-Lys-), -Valine-Arginine- (-Val-Arg-), -Phenylalanine Acid-Citrulline- (-Phe-Cit-), -Phenylalanine-Lysine- (-Phe-Lys-), or -Phenylalanine-Arginine- (-Phe-Arg-). A linker including such dipeptides is cleavable, for example, by cathepsin B.

Where L^{B} is a dipeptide linker as described above, L^{C} may be para-aminobenzyl, para-aminobenzyloxycarbonyl.

L^{D} may be a bisamine cyclization spacer. For example, L^{D} may be selected from —C(O)N(R_{B})(R_{C}) or —C(S)N(R_{B})(R_{C}), wherein L^{D} is bonded to D at the carbon of the C(O) or C(S) group, wherein R_{B} is —C₁-C₂₀ alkylN(R_{D})—, and wherein R_{C} and R_{D} are independently selected from H, C₁-C₂₀ alkyl, and —C₁-C₈ heteroalkyl. Optionally, R_{C} and R_{D} may be independently selected from —C₁-C₂₀ alkyl and —(CH₂CH₂O)ᵣH, where r is an integer from 1 to 4.

Optionally, the electron-withdrawing group is selected from the group consisting of a nitro group, a cyano group, —CF₃ or a halide (e.g. F, Cl, Br, I).

Optionally, the antibody conjugate may comprise a structure selected from:

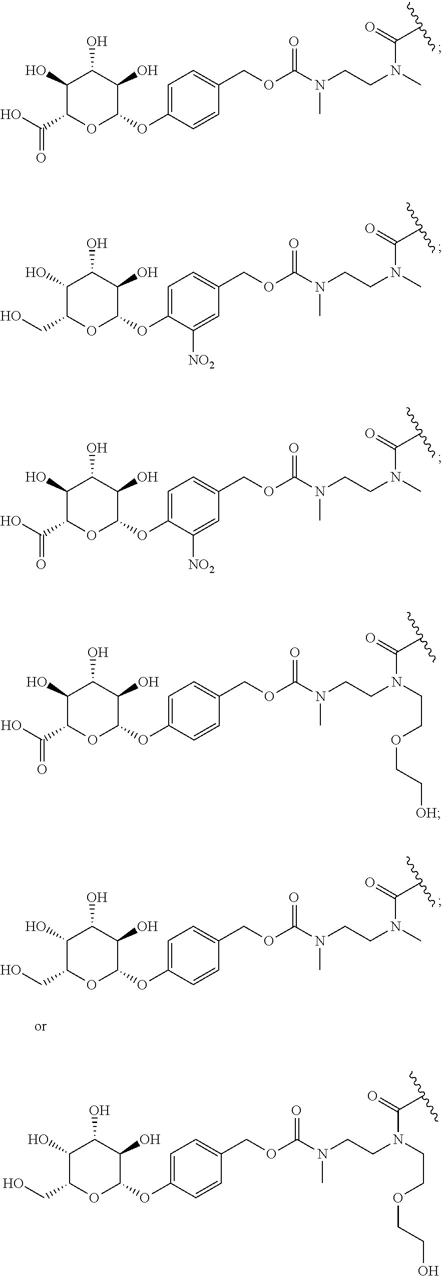

wherein the wavy line indicates the site of attachment to the phenolic oxygen of the first CBI unit.

Optionally, L^{A} may be represented by Formula II:

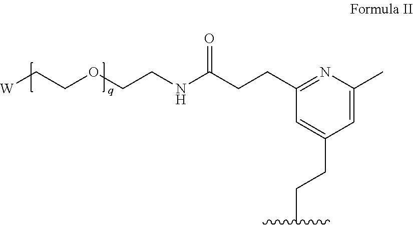

Wherein:
the wavy line indicates a point of attachment to Ab;
W is a coupling group for connecting L^{A} with L^{B}; and
q is an integer from 1 to 20.

For example, W may be an amide group (i.e. —NH—C(O)—) formed by coupling reactions well known in the art.

Linker stability is a critical factor in determining the efficacy and toxicity of an antibody-drug conjugate. An unstable linker can release the cytotoxic payload during blood circulation before it reaches the target site, leading to undesirable systemic toxicities. For example, some linkers, such as the widely-used maleimide attachment method, can suffer from non-specific release of payloads in non-tumorous tissues, leading to off-target toxicity and a limited therapeutic window. Advantageously, connecting groups L^{A} according to Formula II provide a stable connection between the antibody and the drug while allowing efficient cleavage of the drug in tumour cells.

As shown in Formula II, the connecting group L^{A} may include a hydrophilic poly(ethylene glycol) (PEG) spacer. The use of PEG-linkers is known in the art to improve pharmacokinetic profiles. As will be appreciated by the skilled person, the PEG spacer may have different lengths to keep the drug moiety or payload closer or further away from the antibody. For example, n may be 2, 4, 6, 8, 10, 12, 14, 16, 18 or 20. It will also be appreciated by the skilled person that the techniques disclosed herein may be used in conjunction with other poly(alkylene glycol) molecules, such as polypropylene glycol or polyethylene-polypropylene glycol copolymers. Branched or multi-arm poly(alkylene glycol) molecules, including branched or multi-arm PEG molecules, may also be used. Poly(alkylene glycol) molecules that may be used in accordance with the present invention are well known in the art and publicly available, for example from commercially available sources such as Sigma Aldrich.

Optionally, L may be covalently bound to the antibody by a thioether bond. For example, the thioether bond may comprise a sulfur atom of cysteine of the antibody. Cysteine-based conjugation methods offer greater control of drug loading, i.e. the drug-to-antibody ratio (DAR) and homogeneity, compared to lysine conjugation methods. Greater ADC homogeneity is known to be associated with improved pharmacokinetics and efficacy and reduced off-target toxicity. The covalent thioether bond may be formed using existing thiol groups or by introducing thiol groups in a precursor step, for example by reacting one or more functional groups of the antibody to produce a thiol group, or by introducing a thiol group or a precursor thereof into the antibody. By way of example, this may involve the step of introducing a cysteine residue into the antibody at a site where it is desired to bind the linker to the antibody. This may be useful in situations where a convenient cysteine residue for reaction according to the present invention is not present in a starting or wild-type antibody. Conveniently, this may be achieved using site directed mutagenesis of the antibody, the use of which is well established in the art. Advantageously, this may also enable attachment of a drug or other active agent to the antibody in a site-specific manner. This may allow for the preparation of homogeneous ADCs having a defined number of drugs, which is known to improve pharmacokinetics and efficacy and is more desirable from a regulatory perspective.

Optionally, the antibody may be selected from the group consisting of an anti-folate receptor alpha antibody, an anti-CanAg antibody, an anti-B7H3 antibody, an anti-MSLN antibody, an anti-Trop2 antibody, an anti-5T4 antibody, an anti-CD20 antibody, an anti-PSMA antibody, an anti-EGFR antibody, an anti-CD70 antibody, an anti-DLL3 antibody, an anti-ROR1 antibody, an anti-c-MET antibody, an anti-Her3 antibody, an anti-EphA3 antibody, an anti-CD30 antibody, an anti-CD79 antibody, an anti-NaPi3 antibody an anti-CD22 antibody, an anti-CD19 antibody, an anti-CD33 antibody, an anti-Her2 antibody, and an anti-MUC16.

In a second aspect, there is provided a linker-drug intermediate having the formula:

Lp-D

wherein:
Lₚ is a linker precursor comprising a thiol-reactive functional group; and
D is represented by Formula I as described above.

Optionally, Lₚ has the formula:

L^{A}-L^{B}-L^{C}-L^{D}

### Wherein:

L^{A} is a connecting group comprising the thiol-reactive functional group;
L^{B} is an enzyme cleavable linker;
L^{C} is a self-immolative spacer or absent; and
L^{D} is spacer group covalently bound to D.

Optionally, L_{B} may be selected from a β-glucuronide linker or a β-galactoside linker and L^{C} is absent.

Alternatively, L^{B} may be a dipeptide linker selected from -Valine-Citrulline- (-Val-Cit-), -Valine-Alanine- (-Val-Ala-), -Valine-Lysine- (-Val-Lys-), -Valine-Arginine- (-Val-Arg-), -Phenylalanine Acid-Citrulline- (-Phe-Cit-), -Phenylalanine-Lysine- (-Phe-Lys-), or -Phenylalanine-Arginine- (-Phe-Arg-).

When L^{B} is a dipeptide linker as described above, L^{C} may be para-aminobenzyl, para-aminobenzyloxycarbonyl.

Optionally, L^{D} may be selected from —C(O)N(R^{B})(R^{C}) or —C(S)N(R^{B})(R^{C}) and is bonded to D at the carbon of the C(O) or C(S) group, wherein R^{B} is —C₁-C₂₀ alkylN(R_{D})—, and wherein R^{C} and R^{D} are independently selected from H, C₁-C₂₀ alkyl, and —C₁-C₈ heteroalkyl.

Optionally, R^{C} and R^{D} may be independently selected from —C₁-C₂₀ alkyl and —(CH₂CH₂O)ᵣH, where r is an integer from 1 to 4.

Optionally, L^{A} is represented by Formula III:

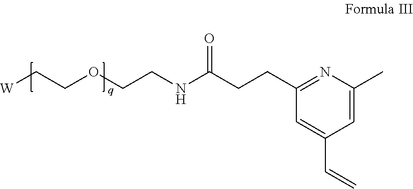

Wherein:
W is a coupling group for connecting L^{A} with L^{B}; and
q is an integer from 1 to 20.

For example, W may be an amide group (i.e. —NH—C(O)—) formed by coupling reactions well known in the art.

Advantageously, vinylpyridine-based linkers in accordance with Formula III have been shown to react selectively and irreversibly with thiol groups on an antibody to form highly stable thioether bonds.

Optionally, the linker-drug intermediate may be selected from:

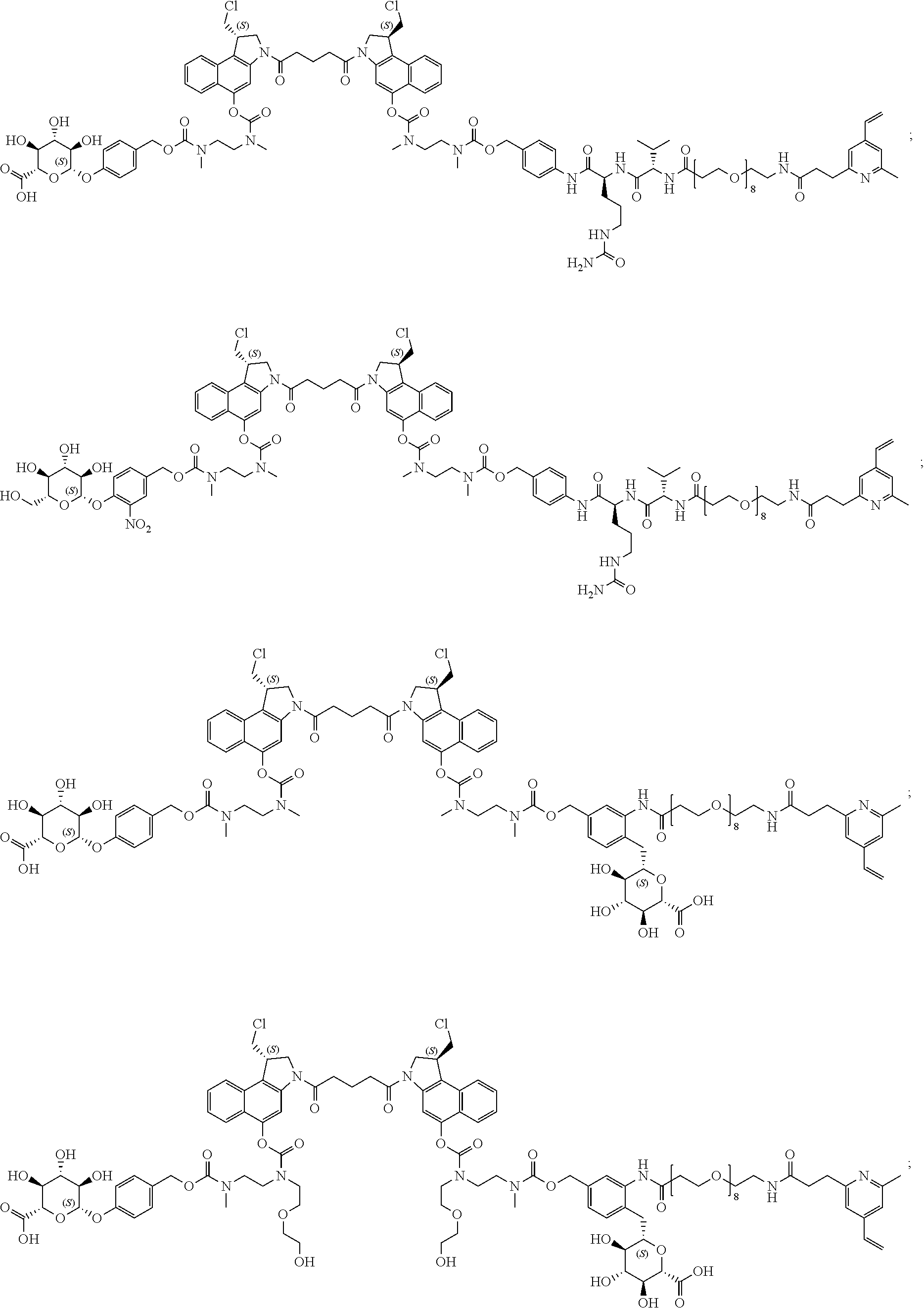

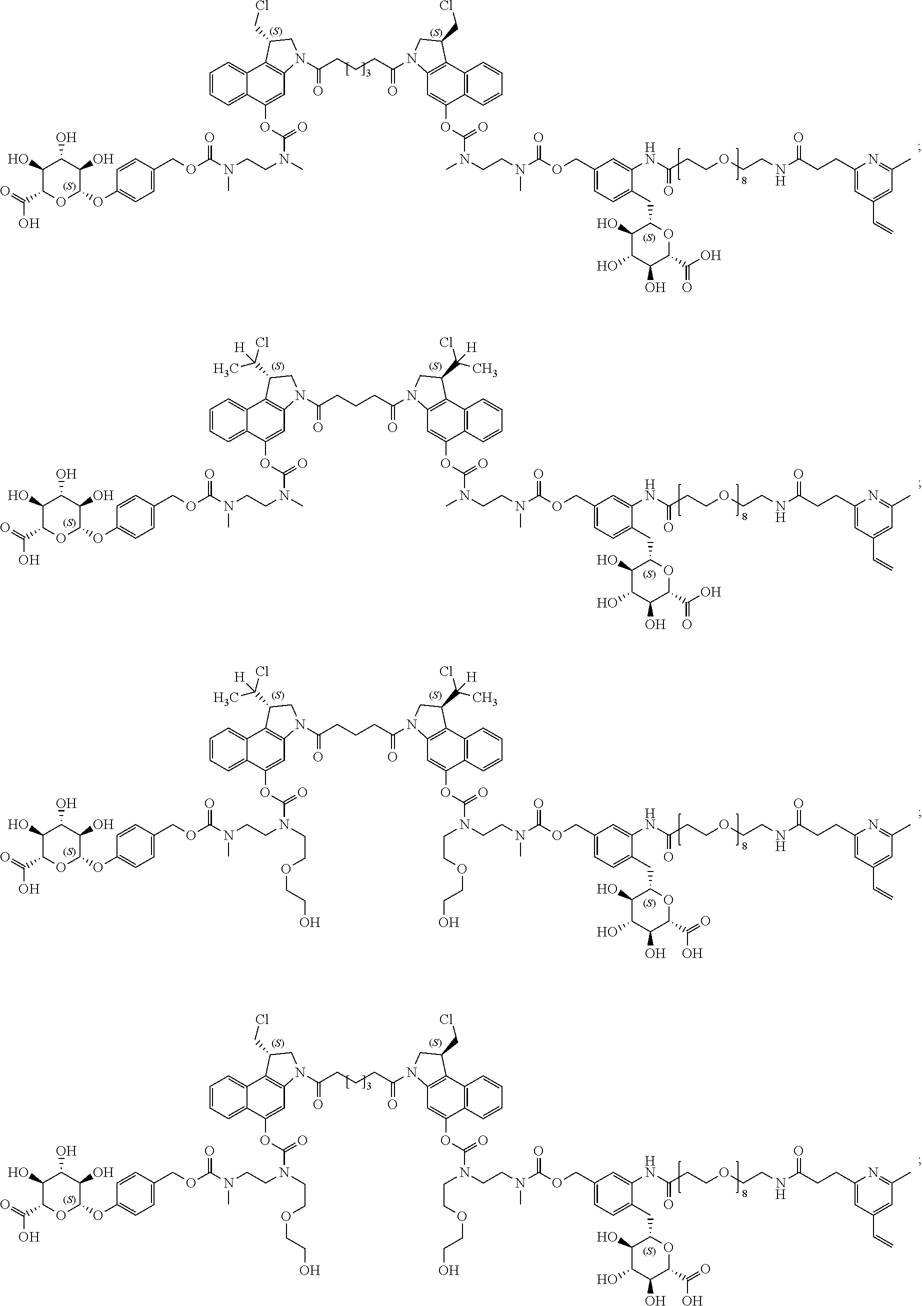

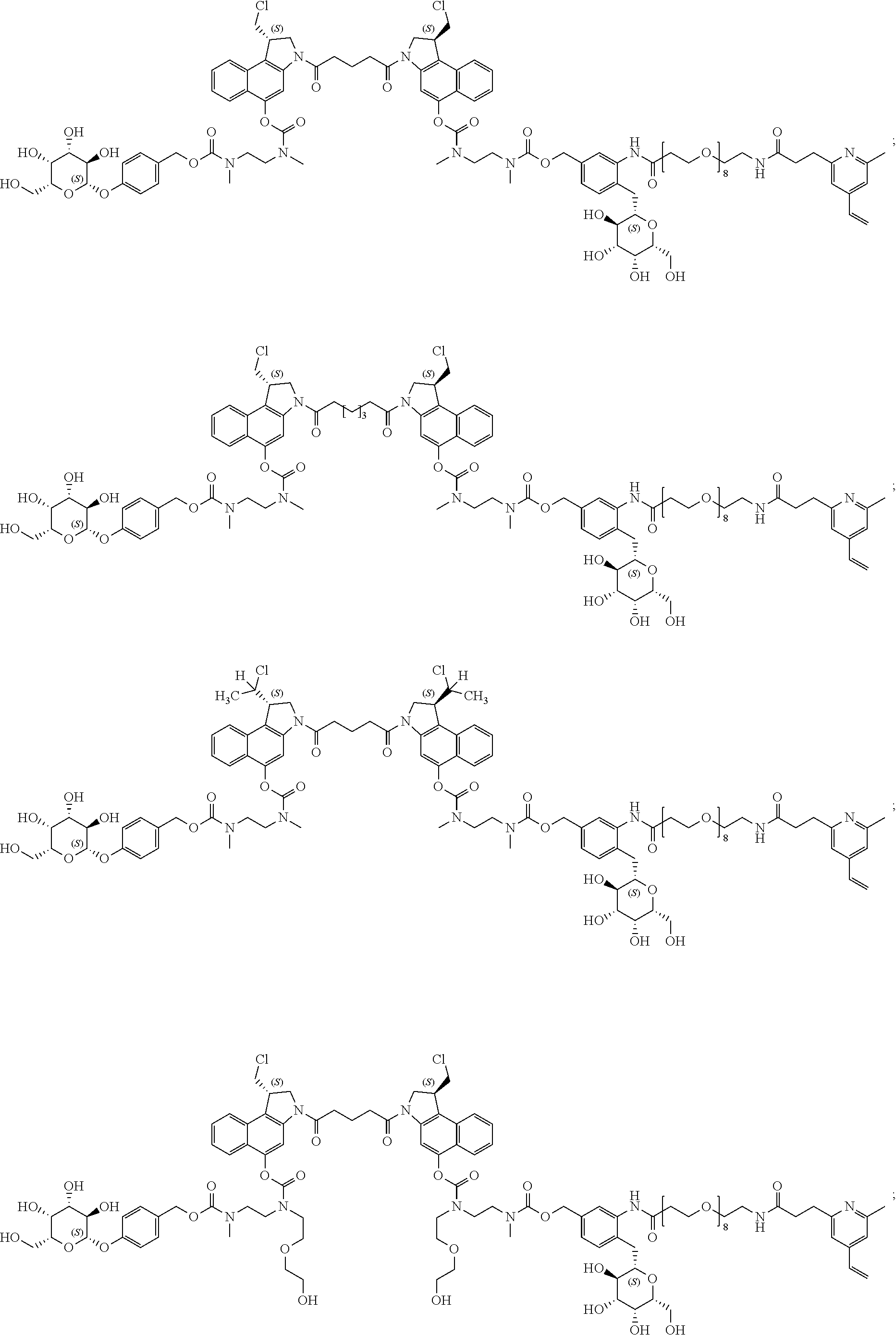

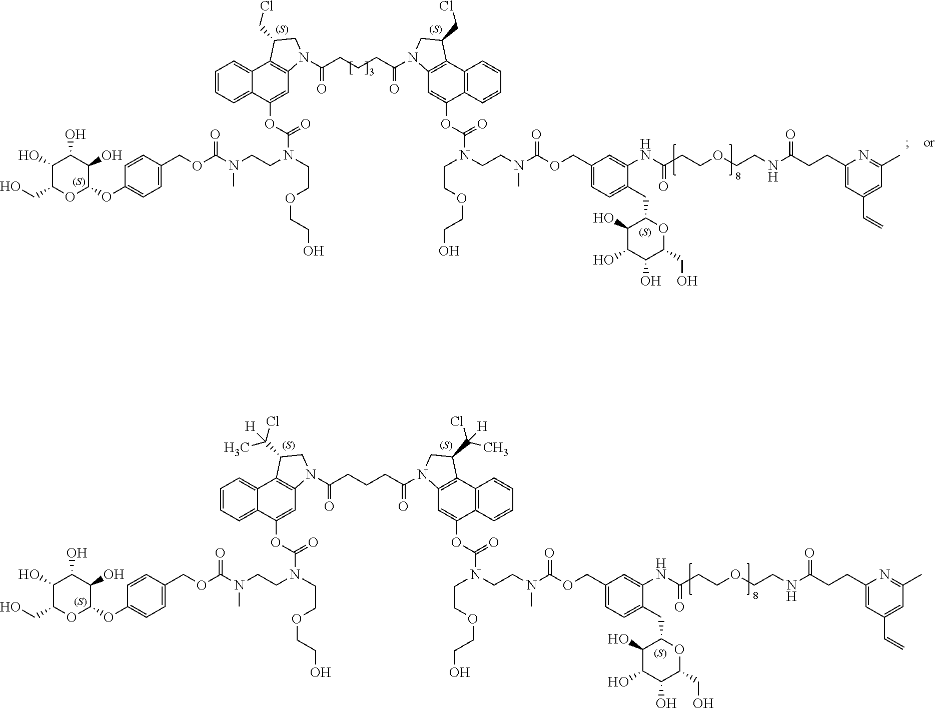

In a third aspect, there is provided a pharmaceutical composition comprising an antibody conjugate according to the first aspect; and one or more pharmaceutically acceptable excipients, diluents, or carriers.

Suitably, the pharmaceutical composition may be for use as a medicament. For example, the medicament may be for use in the treatment of cancer. Optionally, cancer may be selected from the group consisting of ovarian cancer cells, lung cancer cells, uterine cancer cells, testicular choriocarcinoma cells, ependymoma cells, mesothelioma cells, breast cancer cells, colon cancer cells, gastric cancer cells or renal cells carcinoma.

In a fourth aspect, there is provided use of the pharmaceutical composition according to the second aspect in the preparation of a medicament for the treatment of cancer.

In a fifth aspect, there is provided a method of treating cancer in a subject in need thereof, comprising the step of administering a therapeutic amount of the pharmaceutical composition according to the second aspect of the subject.

Optionally, cancer may be selected from the group consisting of ovarian cancer cells, lung cancer cells, uterine cancer cells, testicular choriocarcinoma cells, ependymoma cells, mesothelioma cells, breast cancer cells, colon cancer cells, gastric cancer cells or renal cell carcinoma.

Optionally, the method may comprise, prior to administering the pharmaceutical composition, the step of determining sensitivity of a subject population to ADCs according to the present invention. For example, the method may further comprise assaying a sample from a subject with cancer for the presence of Schlafen 11 (SLFN11). Advantageously, SLFN11 was identified as the most differentially expressed gene that is upregulated in cells sensitive to ADCs according to the present invention.

In another aspect, there is provided a method of producing the antibody-drug conjugate according to the first aspect, comprising contacting the antibody or antigen binding fragment thereof (Ab) with the linker-drug intermediate described above. Optionally, the method may further comprise an initial step of providing a thiol group at one or more desired positions on the antibody or antigen binding fragment thereof (Ab).

Embodiments of the present invention will now be described by way of example and not limitation with reference to the accompanying figures.

### BRIEF DESCRIPTION OF FIGURES

The accompanying drawings illustrate presently exemplary embodiments of the disclosure, and together with the general description given above and the detailed description of the embodiments given below, serve to explain, by way of example, the principles of the disclosure.

FIG. 1 shows a PLRP chromatogram (A214 nm) of an Isumab01-compound 1 ADC prepared in the presence of 20% (v/v) DMA with a DAR of 1.7. Numbers designate the amount of drug conjugated to light (L) or heavy (H) chain;

FIG. 2 shows a PLRP chromatogram (A214 nm) of an Isumab01-compound 1 ADC prepared in the presence of 26 mM DTAB with a DAR of 2.1. Numbers designate the amount of drug conjugated to light (L) or heavy (H) chain;

FIG. 3 shows a PLRP chromatogram (A214 nm) of an Isumab01-compound 2 ADC prepared in the presence of 26 mM DTAB with a DAR of 1.8. Numbers designate the amount of drug conjugated to light (L) or heavy (H) chain;

FIG. 4 shows a PLRP chromatogram (A214 nm) of an Isumab01-compound 2 ADC prepared in the presence of 28% (v/v) propylene glycol+2% (v/v) DMA with a DAR of 2.1. Numbers designate the amount of drug conjugated to light (L) or heavy (H) chain;

FIG. 5 shows a PLRP chromatogram (A214 nm) of an Isumab01-compound 3 ADC with a DAR of 2.1 prepared in the presence of 29% (v/v) propylene glycol+1% (v/v) DMA. Numbers designate the amount of drug conjugated to light (L) or heavy (H) chain;

FIG. 6 shows a PLRP chromatogram (A214 nm) of an Isumab01-compound 4 ADC prepared in the presence of 26 mM DTAB with a DAR of 2.6. Numbers designate the amount of drug conjugated to light (L) or heavy (H) chain;

FIG. 7 shows a PLRP chromatogram (A214 nm) of an Isumab01-compound 5 ADC with a DAR of 2.3 prepared without organic solvent. Numbers designate the amount of drug conjugated to light (L) or heavy (H) chain;

FIG. 8 shows a PLRP chromatogram (A214 nm) of an Isumab04-compound 5 ADC with a DAR of 3.7 prepared without organic solvent. Numbers designate the amount of drug conjugated to light (L) or heavy (H) chain;

FIG. 9 shows a PLRP chromatogram (A214 nm) of an Isumab01-compound 6 ADC with a DAR of 3.3 prepared in the presence of 2% (v/v) DMA. Numbers designate the amount of drug conjugated to light (L) or heavy (H) chain;

FIG. 10 shows a PLRP chromatogram (A214 nm) of an Isumab04-compound 6 ADC with a DAR of 3.7 prepared in the presence of 2% (v/v) DMA. Numbers designate the amount of drug conjugated to light (L) or heavy (H) chain;

FIG. 11 shows a PLRP chromatogram (A214 nm) of an Isumab01-compound 7 ADC with a DAR of 3.5 prepared in the presence of 2% (v/v) DMA. Numbers designate the amount of drug conjugated to light (L) or heavy (H) chain;

FIG. 12 shows a PLRP chromatogram (A214 nm) of an Isumab01-compound 8 ADC with a DAR of 3.0 prepared in the presence of 2% (v/v) DMA. Numbers designate the amount of drug conjugated to light (L) or heavy (H) chain;

FIG. 13 shows a SEC chromatogram (A214 nm) of an Isumab01-compound 1 ADC with a DAR of 2.1 in the presence of 26 mM DTAB. #Monomer (65%); * aggregate (35%);

FIG. 14 shows a SEC chromatogram (A214 nm) of an Isumab01-compound 2 ADC with a DAR of 1.8 in the presence of 26 mM DTAB. #Monomer (73%); * aggregate (27%);

FIG. 15 shows a SEC chromatogram (A214 nm) of an Isumab01-compound 3 ADC with a DAR of 2.1 prepared in the presence of 29% (v/v) propylene glycol+1% (v/v) DMA. #Monomer (86%); * aggregate (14%);

FIG. 16 shows a SEC chromatogram (A214 nm) of an Isumab01-compound 4 ADC with a DAR of 2.6 prepared in in the presence of 26 mM DTAB. #Monomer (73%); * aggregate (27%);

FIG. 17 shows a SEC chromatogram (A214 nm) of an Isumab01-compound 5 ADC with a DAR of 2.3 prepared without organic solvent. #Monomer (98%); * aggregate (2%);

FIG. 18 shows a SEC chromatogram (A214 nm) of an Isumab01-compound 6 ADC with a DAR of 3.3 prepared in the presence of 2% (v/v) DMA. #Monomer (95%); * aggregate (5%);

FIG. 19 shows a SEC chromatogram (A214 nm) of an Isumab04-compound 5 ADC with a DAR of 3.7 prepared without organic solvent. #Monomer (99%); * aggregate (1%);

FIG. 20 shows a SEC chromatogram (A214 nm) of an Isumab04-compound 6 ADC with a DAR of 3.7 prepared in the presence of 2% (v/v) DMA. #Monomer (94%); * aggregate (6%);

FIG. 21 shows a SEC chromatogram (A214 nm) of an Isumab01-compound 7 ADC with a DAR of 3.5 prepared in the presence of 2% (v/v) DMA. #Monomer (94%); * aggregate (6%);

FIG. 22 shows a SEC chromatogram (A214 nm) of an Isumab01-compound 8 ADC with a DAR of 3.0 prepared in the presence of 2% (v/v) DMA. #Monomer (98%); * aggregate (2%);

FIG. 23 shows Jeg-3 cell viability results. A) Isumab01-compound 1, B) Isumab01-compound 2, C) Isumab01-compound 3, D) Isumab01-compound 4;

FIG. 24 shows OVCAR-3 cell viability results. A) Isumab01-compound 1, B) Isumab01-compound 2, C) Isumab01-compound 4;

FIG. 25 shows OV90 cell viability results. A) Isumab01-compound 1, B) Isumab01-compound 2, C) Isumab01-compound 4;

FIG. 26 shows H2110 cell viability results. A) Isumab01-compound 1, B) Isumab01-compound 2, C) Isumab01-compound 4;

FIG. 27 shows Jeg-3 cell viability results. A) Isumab01-compound 5, B) Isumab01-compound 6, C) Isumab01-compound 7;

FIG. 28 shows H2110 cell viability results. A) Isumab01-compound 5, B) Isumab01-compound 6;

FIG. 29 shows Colo 205 cell viability results. A) Isumab04-compound 5 and B) Isumab04-compound 6;

FIG. 30 Volcano plot of gene expression (fold change) versus p-value. SLFN11 was identified as the most differentially expressed gene that is upregulated in sensitive cells;

FIG. 31 shows in vitro stability of A) Adcetris, B) Enhertu, C) Isumab01-compound 5 and D) Isumab01-compound 6 in human IgG-deprived plasma;

FIG. 32 shows in vitro stability of A) Adcetris, B) Enhertu, C) Isumab01-compound 5 and D) Isumab01-compound 6 in Ig-G deprived mouse plasma;

FIG. 33 shows in vitro stability of A) Isumab01-compound 5 and B) Isumab01-compound 6 in whole mouse plasma;

FIG. 34 shows in vivo activity of Isumab01-compound 5, Isumab01-compound 6, ADC non-binding control-compound 5 and ADC non-binding control-compound 6 in Jeg-3 xenograft (A). Body weight changes are shown in B;

FIG. 35A shows the synthesis of compound 20;

FIG. 35B shows the synthesis of compound 1;

FIG. 36A shows the synthesis of compound 39;

FIG. 36B shows the synthesis of compound 2;

FIG. 37A shows the synthesis of compound 53;

FIG. 37B shows the synthesis of compound 61;

FIG. 37C shows the synthesis of compound 3;

FIG. 38A shows the synthesis of compound 17;

FIG. 38B shows the synthesis of compound 78;

FIG. 38C shows the synthesis of compound 5;

FIG. 39A shows the synthesis of compound 93;

FIG. 39B shows the synthesis of compound 99;

FIG. 39C shows the synthesis of compound 6;

FIG. 40 shows the synthesis of compound 7;

FIG. 41A shows the synthesis of compounds 107 and 109; and

FIG. 41B shows the synthesis of compound 8.

### DETAILED DESCRIPTION

The present invention relates generally to antibodies conjugated to cyclopropylbenzoindole (CBI) dimer drug moieties to form antibody-drug conjugates and to compositions comprising the antibody-drug conjugates. Methods of making the antibody-drug conjugates are also provided. Methods of using the antibody-drug conjugates and compositions, including for the treatment of cancer, are further provided.

### DEFINITIONS AND ABBREVIATIONS

Unless stated otherwise, the following terms and phrases as used herein are intended to have the following meanings. When trade names are used herein, the trade name includes the product formulation, the generic drug, and the active pharmaceutical ingredient(s) of the trade name product, unless otherwise indicated by context.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. References to antibodies include immunoglobulins whether natural or partly or wholly synthetically produced. The term also covers any polypeptide or protein comprising an antigen binding domain. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, nanobodies, single chain antibodies, bispecific and multispecific antibodies formed from antibody fragments.

An antibody “specifically binds” to an epitope or antigenic molecule, which means that the antibody interacts or associates more frequently, more rapidly, with greater duration, with greater affinity, or with some combination of the foregoing to an epitope or antigenic molecule than alternative substances, including unrelated proteins. In specific embodiments, “specifically binds” means, for instance, that an antibody binds to a protein with a Ko of approximately 0.1 mM or less, but more usually, less than about 1 μM. In specific embodiments, “specifically binds” means that an antibody binds to a protein at times with a K_{D} of approximately 0.1 μM or less, and at other times, with a K_{D} of approximately 0.01 μM or less.

The terms “cancer” and “cancerous” refer to or describe the physiological condition in mammals in which a population of cells is characterised by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, fallopian tube cancer, gastric cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

“Tumour” refers to any mass of tissue that results from excessive cell growth or proliferation, either benign (noncancerous) or malignant (cancerous) including precancerous lesions.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical composition” refers to a preparation which is in such form as to permit the biological activity of the active ingredient to be effective, and which contains no additional components which are unacceptably toxic to a subject to which the formulation would be administered. Such formulation can be sterile.

An “effective amount” as disclosed herein is an amount sufficient to carry out a specifically stated purpose. An “effective amount” can be determined empirically and in a routine manner, in relation to the stated purpose.

The term “therapeutically effective amount” refers to an amount of an ADC or other drug effective to “treat” a disease or disorder in a subject or mammal. In the case of cancer, the therapeutically effective amount of the drug can reduce the number of cancer cells; reduce the tumour size; inhibit (i.e., slow to some extent and in a certain embodiment, stop) cancer cell infiltration into peripheral organs; inhibit (i.e., slow to some extent and in a certain embodiment, stop) tumour metastasis; inhibit, to some extent, tumour growth; and/or relieve to some extent one or more of the symptoms associated with cancer. See the definition of “treating” below. To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for cancer according to the methods of the present invention if the patient shows one or more of the following: a reduction in the number of or complete absence of cancer cells; a reduction in the tumour size; inhibition of or an absence of cancer cell infiltration into peripheral organs including, for example, the spread of cancer into soft tissue and bone; inhibition of or an absence of tumour metastasis; inhibition or an absence of tumour growth; relief of one or more symptoms associated with specific cancer; reduced morbidity and mortality; improvement in quality of life; reduction in tumorigenicity, tumorigenic frequency, or tumorigenic capacity, of a tumour; reduction in the number or frequency of cancer stem cells in a tumour; differentiation of tumorigenic cells to a non-tumorigenic state; or some combination of effects.

“Prodrug” refers to a compound that is metabolised, for example hydrolysed, in the host after administration to form a biologically active molecule. Typical examples of prodrugs include compounds that have biologically labile or cleavable protecting groups on a functional moiety of the active compound.

“Self-immolative spacer” refers to a moiety that spaces and covalently links together two or more components and degrades spontaneously in response to specific stimuli.

“Electron-withdrawing group” refers to a group that draws electron density from neighbouring atoms towards itself, typically by resonance or inductive effects.

The term “alkyl” by itself or as part of another term refers to a straight chain or branched, saturated hydrocarbon having the indicated number of carbon atoms (e.g., “C₁-C₈” alkyl refers to an alkyl group having from 1 to 8 carbon atoms). Alkyl groups typically comprise from 1 to 20 carbon atoms, preferably from 1 to 8 carbon atoms, and more preferably from 1 to 4 carbon atoms. When the number of carbon atoms is not indicated, the alkyl group has from 1 to 8 carbon atoms. Representative straight chain C₁-C₈ alkyls include, but are not limited to, methyl, ethyl, n-propyl, n-butyl, n-pentyl, n-hexyl, n-heptyl and n-octyl; while branched C₁-C₈ alkyls include, but are not limited to, -isopropyl, -sec-butyl, -isobutyl, -tent-butyl, -isopentyl, and -2-methylbutyl; unsaturated C2-C8 alkyls include, but are not limited to, vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexyl, 2-hexyl, 3-hexyl, acetylenyl, propynyl, 1-butynyl, 2-butynyl, 1-pentynyl, 2-pentynyl and 3-methyl-1-butynyl. Reference to “alkyl” herein refers to unsubstituted and substituted moieties as described above.

The term “alkylene,” by itself or as part of another term, refers to a saturated, branched or straight chain or cyclic hydrocarbon radical of the stated number of carbon atoms, typically 1-18 carbon atoms, and having two monovalent radical centers derived by the removal of two hydrogen atoms from the same or two different carbon atoms of a parent alkane. Examples include methylene, ethylene, propylene, butylene, pentylene, hexylene, heptylene, ocytylene, nonylene and decalene. Reference to “alkylene” herein refers to unsubstituted and substituted moieties as described above.

An “alkene” moiety refers to a group consisting of at least two carbon atoms and at least one carbon-carbon double bond.

The term “heteroalkyl,” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive. Heteroalkyl groups typically comprise from 1 to 15 carbon atoms, preferably from 1 to 12 carbon atoms, more preferably from 1 to 8 carbon atoms, and most preferably from 1 to 4 carbon atoms. Reference to “heteroalkyl” herein refers to unsubstituted and substituted moieties as described above. Unless otherwise indicated, the term “heteroalkylene” by itself or as part of another substituent means a divalent group derived from heteroalkyl (as discussed above). For heteroalkylene groups, heteroatoms can also occupy either or both of the chain termini. Reference to “heteroalkylene” herein refers to unsubstituted and substituted moieties as described above.

An “alkyne” moiety refers to a group consisting of at least two carbon atoms and at least one carbon-carbon triple bond. The alkyl moiety, whether saturated or unsaturated, may be branched, straight chain, or cyclic.

The term “aryl” or “aromatic ring” refers to a monoradical of an aromatic cyclic hydrocarbon. Preferably, the aryl group contains 3 to 14 (e.g. 5 to 10, such as 5, 6 or 10) carbon atoms, more preferably 6 to 10 carbon atoms. These can be arranged in one ring, e.g. phenyl, or two or more condensed rings (e.g. naphthyl). Preferably aryl refers to a monocyclic ring containing 6 carbon atoms or an aromatic bicyclic ring system containing 10 carbon atoms. In some embodiments, the aryl is unsubstituted in some embodiments the aryl is substituted.

The term “cycloalkyl” as used herein refers to saturated or unsaturated, non-aromatic cycloalkyl comprising 1, 2 or more rings. Examples include cyclopropyl, cyclo-butyl, cyclopentyl, cyclopentenyl, cyclopentadienyl, cyclohexonyl, etc.

The term “acyl” as used herein refers to a functional group with the general formula R_{ac}—C(O)H, wherein R_{ac} refers to an optionally substituted hydrocarbon radical, in particular, a hydrocarbon chain having C₁-C₈ carbon atoms.

The term “alkylsulfonyl” or “arylsulfonyl” refer to alkyl or aryl groups containing a SO₂ residue.

The term “Nitro” refers to the —NO₂ radical.

The following abbreviations are used herein and have the indicated definitions:

| ABBREVIATION | DEFINITION |
|---|---|
| Ac | Acetyl (protecting group) |
| AC₂O | Acetic anhydride |
| AcOH | Acetic acid |
| Boc | Tert-Butyloxycarbonyl (protecting group) |
| Cbz | N-Benzyloxycarbonyl (protecting group) |
| DCM | Dichloromethane |
| DIEA | Diethylamine |
| DIPEA | N,N-Diisopropylethylamine |
| DMA | N,N-Dimethylacetamide |
| DMAP | 4-(Dimethylamino)pyridine |
| DMF | N,N-Dimethylformamide |
| DMSO | Dimethyl sulfoxide |
| EDCI | N-Ethyl-N′-(3-dimethylaminopropyl)-carbodiimide hydrochloride |
| ESI | Electrospray ionisation |
| Et2O | Diethyl ether |
| EtOAc | Ethyl acetate |
| Ac | Acetyl (protecting group) |
| HATU | N-[(Dimethylamino)-1H-1,2,3-triazolo-[4,5-b]28yridine-1- |
| | ylmethylene]-N-methylmethanaminium hexafluorophosphate |
| | N-oxide |
| h | hours |
| LCMS | Liquid chromatography mass spectrometry |
| HPLC | High performance liquid chromatography |
| m/z | Mass to charge ratio |
| MeOH | Methanol |
| NMR | Nuclear magnetic resonance (spectroscopy) |
| O/N | Overnight |
| ProAlk | Protein Alkylator |
| PrOH | Propanol |
| R_{f} | Retention factor |
| RP | Reverse phase |
| TEA | Triethylamine |
| THF | Tetrahydrofuran |
| TLC | Thin layer chromatography |

The disclosure provides antibody-drug conjugates comprising CBI-dimers, and methods for using the same to treat cancer. Advantageously, ADCs of the present invention exhibit high efficacy as well as improved safety profiles compared to marketed ADCs.

The antibody used herein recognises an antigen that is natively expressed or overexpressed by target cells, e.g., cancer cells, and can function as a targeting agent to deliver drug moieties to cancer cells with a high degree of specificity. When the antibody binds to the antigen, the antigen-conjugate forms a complex, is internalized, and ultimately enters the lysosome, and the linker between the drug moiety and the antibody is cleaved to release the drug moiety and thereby providing a cytotoxic effect.

The antibody may be, for example, selected from the group consisting of an anti-folate receptor alpha antibody, an anti-CanAg antibody, an anti-171H3 antibody, an anti-MSLN antibody, an anti-Trop2 antibody, an anti-5T4 antibody, an anti-CD20 antibody, an anti-PSMA antibody, an anti-EGFR antibody, an anti-CD70 antibody, an anti-DLL3 antibody, an anti-ROR1 antibody, an anti-c-MET antibody, an anti-Her3 antibody, an anti-EphA3 antibody, an anti-CD30 antibody, an anti-CD79 antibody, an anti-NaPi3 antibody, an anti-CD22 antibody, an anti-CD19 antibody, an anti-CD33 antibody, an anti-Her2 antibody, an anti-MUC16, but is not limited thereto.

In a specific example, the following antibodies were used for ADC construction: anti-folate receptor alpha humanised antibody (Isumab01) and anti-CanAg humanised antibody (Isumab04).

### Isumab01 Sequence

### SEQ ID NO: 1—Heavy Chain

QVQLVQSGAEVKKPGASVKVSCKASGYSFTGYFMNWVRQAPGQGL
EWMGRIHPYDGDTFYAQKFQGRVTMTVDKSISTAYMELSRLRSDD
TVVYYCTRYDGSRAMDYWGQGTTVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSREEMT
KNQVSLTCLVKGFYPSDIAVEWESNGQPENNYKTTPPVLDSDGSF
FLYSKLTVDKSRWQQGNVFSCSVMHEALHNHYTQKSLSLSPG

### SEQ ID NO: 2—Light Chain

EIVLTQSPATLSLSPGERATLSCRASQSVSFAGTSLLHWYQQKPG
QAPRLLIYRASNLETGIPARFSGSGSKTDFTLTISSLEPEDFAVY
YCQQSREYPYTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC

### Isumab04 Sequence

### SEQ ID NO: 3—Heavy Chain

MDPKGSLSWRILLFLSLAFELSYGQVQLVQSGAEVKKPGASVKVS
CKASDYTFTYYGINWVRQAPGQGLEWMGWIDTTTGEPNYAQKLQG
RVTFTLDTSASTAYMELRSLRSDDTAVYYCARRGPYNWYFDVWGQ
GTLVTVSSASTKGPSVFPLAPSSKSTSGGTAALGCLVKDYFPEPV
TVSWNSGALTSGVHTFPAVLQSSGLYSLSSVVTVPSSSLGTQTYI
CNVNHKPSNTKVDKKVEPKSCDKTHTCPPCPAPELLGGPSVFLFP
PKPKDTLMISRTPEVTCVVVDVSHEDPEVKFNWYVDGVEVHNAKT
KPREEQYNSTYRVVSVLTVLHQDWLNGKEYKCKVSNKALPAPIEK
TISKAKGQPREPQVYTLPPSREEMTKNQVSLTCLVKGFYPSDIAV
EWESNGQPENNYKTTPPVLDSDGSFFLYSKLTVDKSRWQQGNVFS
CSVMHEALHNHYTQKSLSLSPG

### SEQ ID NO: 4—Light Chain

METDTLLLWVLLLWVPGSTGDIVMTQTPLSLPVTPGEPASISCRS
SKSLLHSNGNTYLYWYLQKPGQSPQLLIYRMSNRASGVPDRFSGS
GSGTDFTLKISRVEAEDVGVYYCLQHLEYPFTFGPGTKVDIKRTV
AAPSVFIFPPSDEQLKSGTASVVCLLNNFYPREAKVQWKVDNALQ
SGNSQESVTEQDSKDSTYSLSSTLTLSKADYEKHKVYACEVTHQG
LSSPVTKSFNRGEC

The antibody-drug conjugates of the invention may comprise cysteine engineered antibodies where one or more amino acids of a wild-type or parent antibody are replaced with a cysteine amino acid. Conveniently, this may be achieved using site directed mutagenesis of the antibody, the use of which is well established in the art. Any form of antibody may be so engineered, i.e. mutated. For example, a parent Fab antibody fragment may be engineered to form a cysteine engineered Fab, referred to herein as “ThioFab.” Similarly, a parent monoclonal antibody may be engineered to form a “ThioMab.” It should be noted that a single site mutation yields a single engineered cysteine residue in a ThioFab, while a single site mutation yields two engineered cysteine residues in a ThioMab, due to the dimeric nature of the IgG antibody.

The engineered cysteine thiols may react with linker reagents or the linker-drug intermediates of the present invention which have thiol-reactive, electrophilic groups such as vinylpyridine, maleimide or alpha-halo amides to form ADC with cysteine engineered antibodies (ThioMabs) and the drug (D) moiety. The location of the drug moiety can thus be designed, controlled, and known. The drug loading can be controlled since the engineered cysteine thiol groups typically react with thiol-reactive linker reagents or linker-drug intermediates in high yield. Engineering an antibody to introduce a cysteine amino acid by substitution at a single site on the heavy or light chain gives two new cysteines on the symmetrical antibody. A drug loading near 2 can be achieved and near homogeneity of the conjugation product ADC.

The “drug-antibody ratio” (DAR) in an antibody conjugate or composition of the invention is defined as the molar ratio between the drug moieties in the conjugate or composition and the antibodies in the conjugate or composition. Where an antibody has more than one site of attachment, more than one drug moiety may be linked to each antibody. In some instances, a mixture is obtained comprising more than one antibody-drug conjugate (ADC) molecules. The drug-antibody ratios of the antibody-drug conjugates can be measured by analytical methods known in the art, for example, as described below. In some embodiments, the antibody conjugates have an average DAR of about 1 to about 6, about 1 to about 5, about 1 to about 4, about 1 to about 6, or about 2 to about 4.

The antibody conjugates according to the present invention may be useful in a variety of applications including, but not limited to, therapeutic treatment methods, such as the treatment of cancer. In certain embodiments, the agents are useful for inhibiting tumour growth, inducing differentiation, reducing tumour volume, and/or reducing the tumorigenicity of a tumour. The methods of use may be in vitro, ex vivo, or in vivo methods. In certain embodiments, the disease treated with the antibody conjugate or compositions comprising the antibody conjugate is cancer.

The present invention provides for methods of treating cancer comprising administering a therapeutically effective amount of the antibody conjugates or compositions thereof to a subject (e.g., a subject in need of treatment). In certain embodiments, the cancer is a cancer selected from the group consisting of ovarian cancer cell, lung cancer cell, uterine cancer cell, testicular choriocarcinoma cell, ependymoma cell, mesothelioma cell, breast cancer cell, colon cancer cell, or renal cell carcinoma. In certain embodiments, the cancer is ovarian cell cancer. In certain embodiments, the subject is a human.

The pharmaceutical compositions of the present invention can be administered in any number of ways for either local or systemic treatment. Administration can be pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration.

For the treatment of the disease, the appropriate dosage of an antibody or agent of the present invention depends on the type of disease to be treated, the severity and course of the disease, the responsiveness of the disease, whether the antibody conjugate administered for therapeutic or preventative purposes, previous therapy, patient's clinical history, and so on all at the discretion of the treating physician. The antibody conjugate or compositions thereof can be administered one time or over a series of treatments lasting from several days to several months, or until a cure is effected or a diminution of the disease state is achieved (e.g. reduction in tumour size). Optimal dosing schedules can be calculated from measurements of drug accumulation in the body of the patient and will vary depending on the relative potency of an individual antibody or agent. The administering physician can easily determine optimum dosages, dosing methodologies and repetition rates.

### Experimental Data and Discussion

The invention is further illustrated by the following examples, which are not to be construed in any way as imposing limitations to the scope of this invention as defined by the appended claims.

The following experimental data was produced utilising the seco-CBI dimeric linker-drug molecules identified as, and referred to herein as, compounds 1 to 8, as shown below.

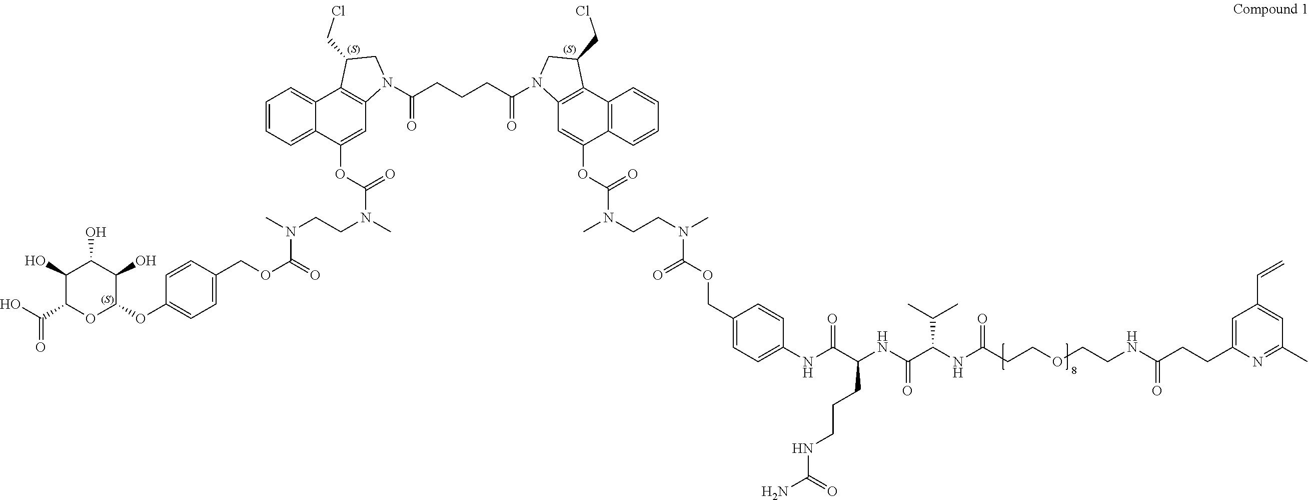

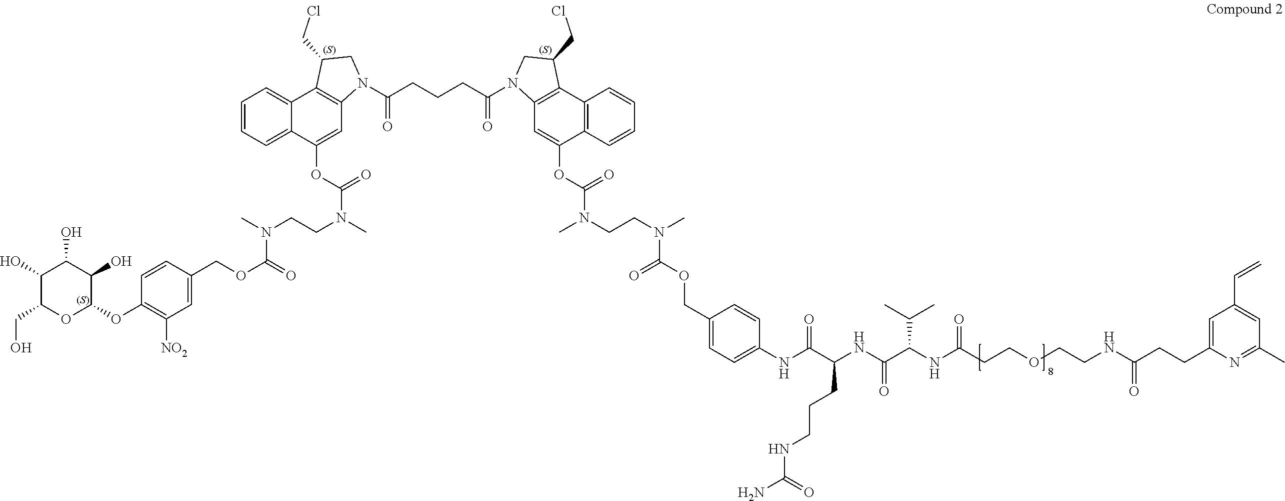

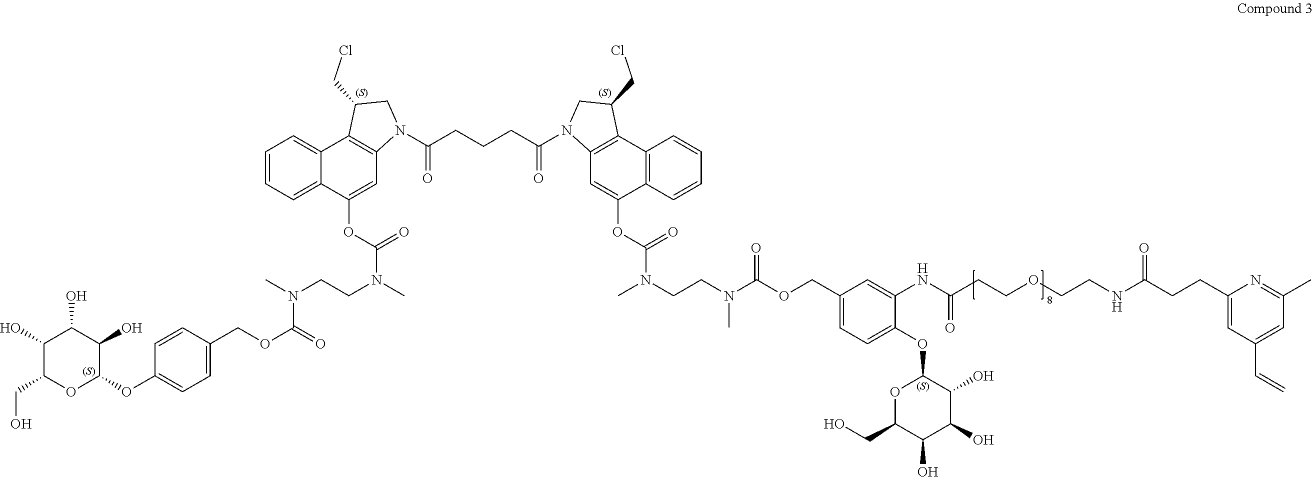

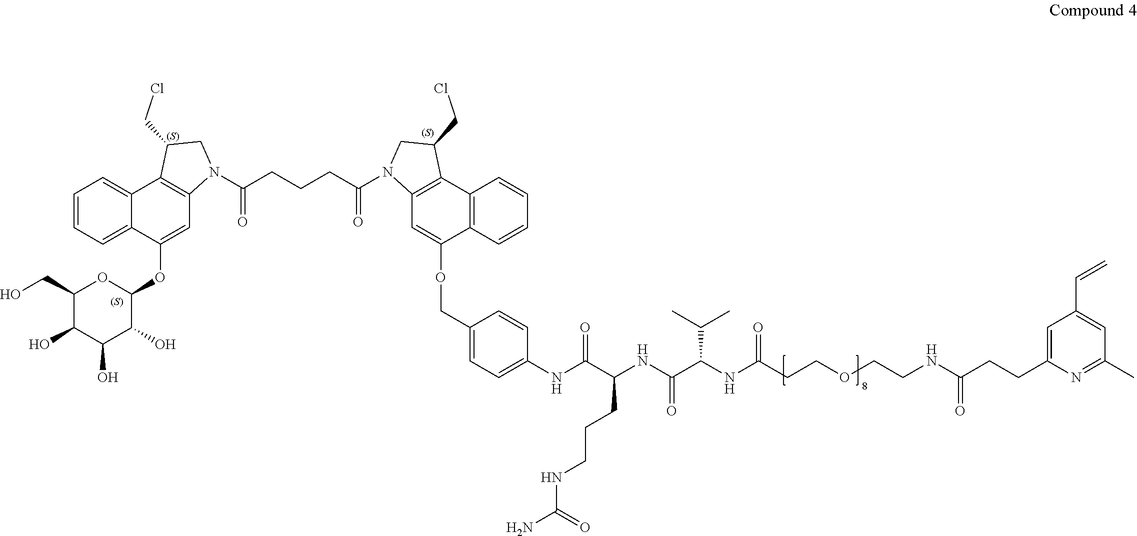

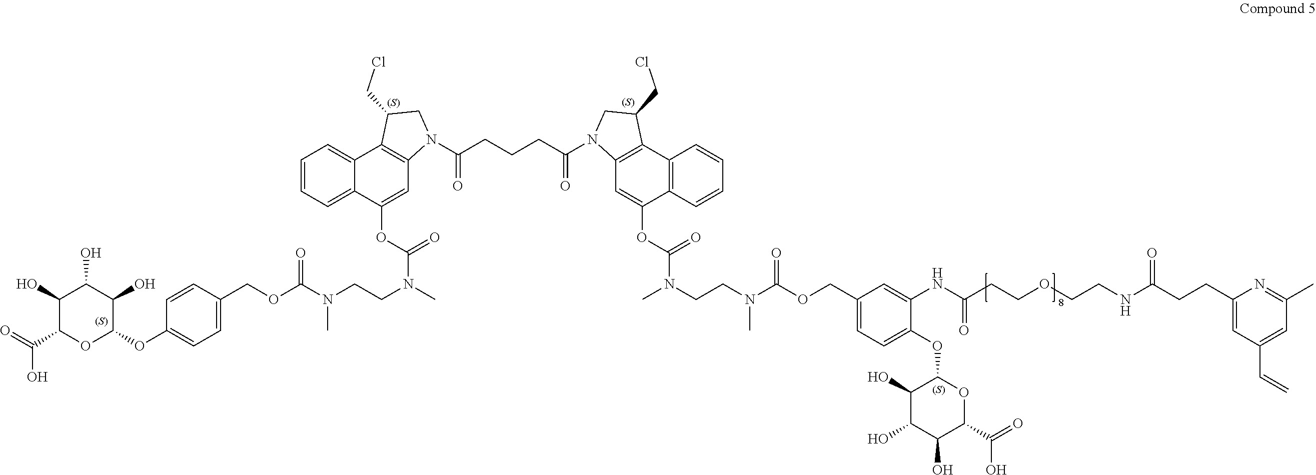

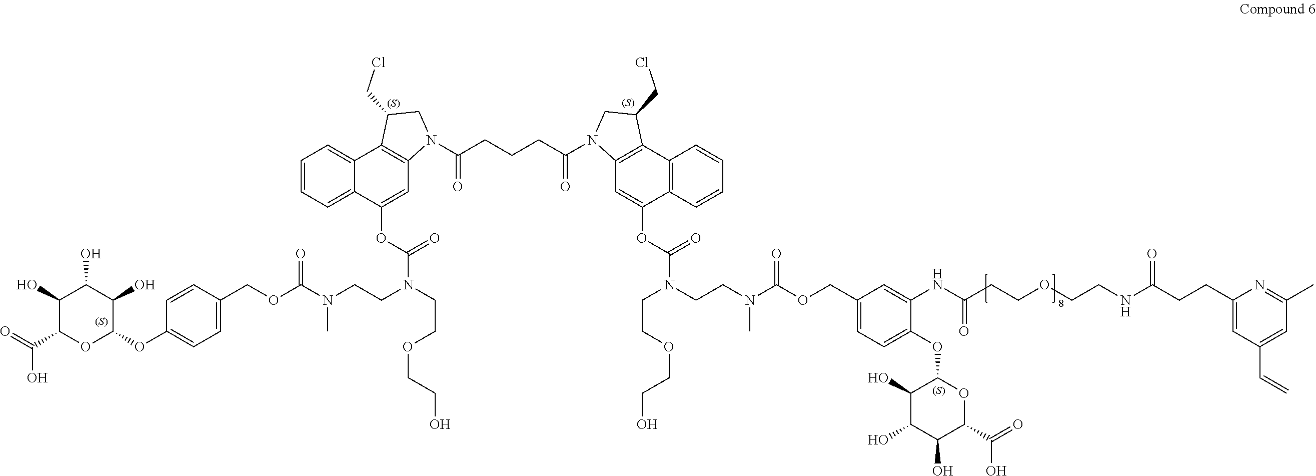

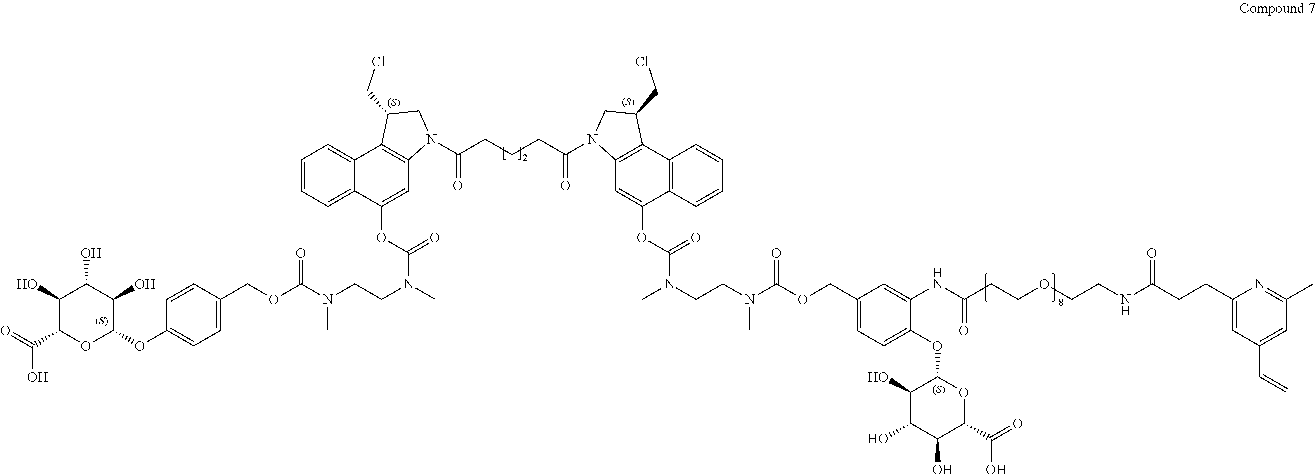

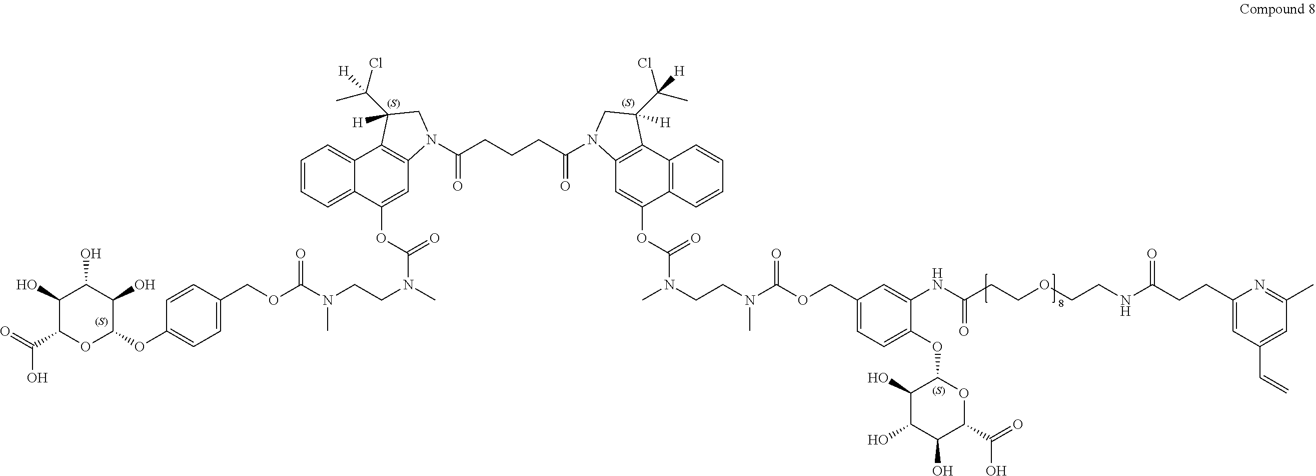

### Synthesis of Payloads and Linker-Payloads

The compounds and conjugates of the invention can be made using the synthetic procedures outlined below.

(2S,3R,4S,5S,6S)-2-(4-(hydroxymethyl)phenoxy)-6-(methoxycarbonyl)tetrahydro-2H-pyran-3,4,5-triyl triacetate (9), 4-((S)-2-((S)-2-amino-3-methylbutanamido)-5-ureidopentanamido)benzyl tert-butyl ethane-1,2-diylbis(methylcarbamate) (26), lithium 3-(6-methyl-4-vinylpyridin-2-yl)propanoate (21), tert-butyl (2-((chlorocarbonyl)(methyl)amino)ethyl)(methyl)carbamate (33) and (2R,3S,4S,5R,6S)-2-(acetoxymethyl)-6-(4-(((methyl(2-(methylamino)ethyl)carbamoyl)oxy)methyl)-2-nitrophenoxy)tetrahydro-2H-pyran-3,4,5-triyl triacetate (37) have been reported previously, for example, in Ghosh et al., Tetrahedron Lett. (1997), 38, 8795-8798), CN109464654A, WO2016067021, US20180072730A1, H. J. Schuster et. al., Org. Biomol. Chem. (2010), 8, 1833-1842, the contents of which are hereby incorporated by reference in their entirety.

(2R,3R,4S,5S,6S)-2-bromo-6-(methoxycarbonyl)tetrahydro-2H-pyran-3,4,5-triyl triacetate (66), tert-butyl methyl(2-(methylamino)ethyl)carbamate (11), tert-butyl(S)-5-(benzyloxy)-1-(chloromethyl)-1,2-dihydro-3H-benzo[e]indole-3-carboxylate (31), (S)-tert-butyl 1-(chloromethyl)-5-hydroxy-1H-benzo[e]indole-3(2H)-carboxylate (14) and tert-butyl (S)-5-(benzyloxy)-1-((R)-1-chloroethyl)-1,2-dihydro-3H-benzo[e]indole-3-carboxylate (103) are commercially available.

### Preparation of Compound 1

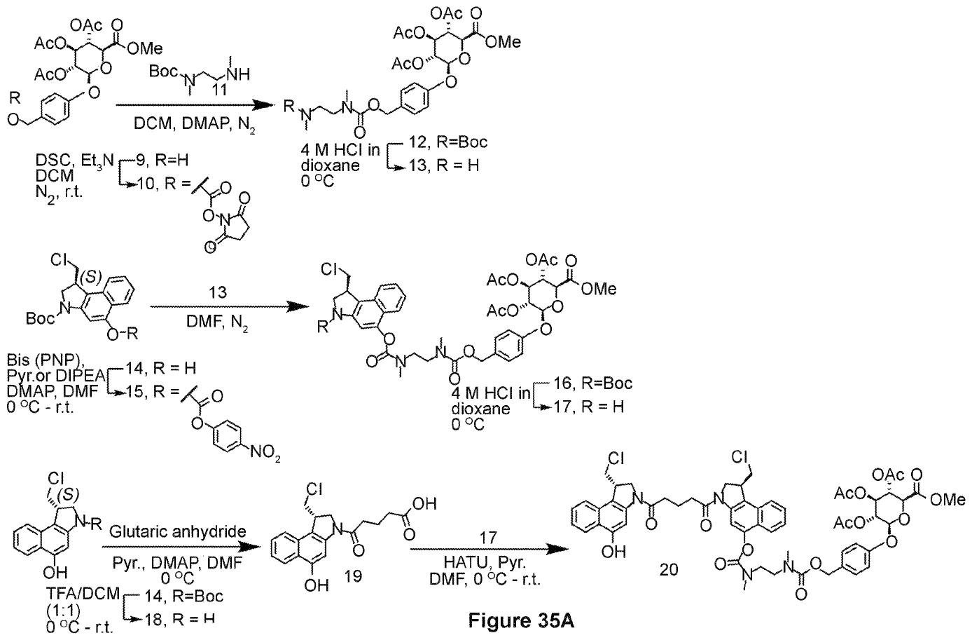

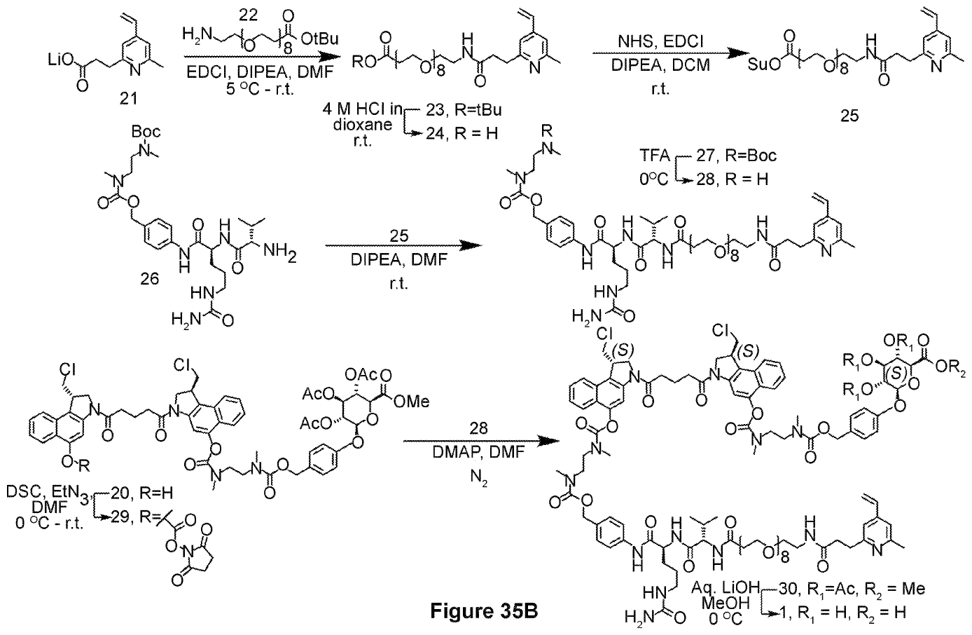

(2S,3S,4S,5R,6S)-6-(4-((((2-(((((S)-1-(chloromethyl)-3-(5-((S)-1-(chloromethyl)-5-(((2-((((4-((2S,5S)-5-isopropyl-37-(6-methyl-4-vinylpyridin-2-yl)-4,7,35-trioxo-2-(3-ureidopropyl)-10,13,16,19,22,25,28,31-octaoxa-3,6,34-triazaheptatriacontanamido)benzyl)oxy)carbonyl)(methyl)amino)ethyl)(methyl)carbamoyl)oxy)-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)phenoxy)-3,4,5-trihydroxytetrahydro-2H-pyran-2-carboxylic acid) (1) was prepared according to the schemes shown in FIGS. 35A and 35B.

(2S,3R,4S,5S,6S)-2-(4-(hydroxymethyl)phenoxy)-6-(methoxycarbonyl)tetrahydro-2H-pyran-3,4,5-triyl triacetate (9) (750 mg, 1.70 mmol) and N,N′-disuccinimidyl carbonate (654 mg, 2.55 mmol) were combined under nitrogen and suspended in anhydrous dichloromethane (80 mL). Triethylamine (356 μL, 2.56 mmol) was added and the reaction was stirred at room temperature. After 1 h, ted-butyl methyl(2-(methylamino)ethyl)carbamate (11) (545 mg, 2.90 mmol) in anhydrous dichloromethane (0.4 mL), and DMAP (354 mg, 2.90 mmol) were added. Stirring was continued for 1 h at room temperature before the reaction was concentrated in vacuo and purified by flash chromatography (silica gel; 0-5% methanol in dichloromethane) to give (2S,3S,4S,5R,6S)-2-(methoxycarbonyl)-6-(4-(4,7,10,10-tetramethyl-3,8-dioxo-2,9-dioxa-4,7-diazaundecyl)phenoxy)tetrahydro-2H-pyran-3,4,5-triyl triacetate (12) as a white solid (810 mg, 73%). ¹H NMR (CDCl₃, 300 MHz) δ (ppm): 7.31 (2H, d, J=9.0 Hz), 6.98 (2H, d, J=9.0 Hz), 5.32 (3H, m), 5.14 (1H, d, J=9.0 Hz), 5.05 (2H, s), 4.19 (1H, m, J=6.0 and 3.0 Hz), 3.73 (3H, s), 3.35 (3H, broad s), 2.94 (2H, s), 2.87 (1H, broad s), 2.78 (1H, broad s), 2.05 (15H, s), 1.26 (9H, s); ESI-LCMS: m/z=677.2 [M+Na]⁺. calculated for C₃₀H₄₁N₂O₁₄Na=677.2.

(2S,3S,4S,5R,6S)-2-(methoxycarbonyl)-6-(4-(4,7,10,10-tetramethyl-3,8-dioxo-2,9-dioxa-4,7-diazaundecyl)phenoxy)tetrahydro-2H-pyran-3,4,5-triyl triacetate (12) (347 mg, 0.53 mmol) was dissolved in 4 N hydrochloric acid in 1,4-dioxane (2.65 mL) at 0° C and stirred for 50 minutes. Upon completion, the reaction was concentrated under reduced pressure to give crude (2S,3S,4S,5R,6S)-2-(methoxycarbonyl)-6-(4-(((methyl(2-(methylamino)ethyl)carbamoyl)oxy)methyl)phenoxy)tetrahydro-2H-pyran-3,4,5-triyl triacetate (13), which was used directly without purification. Simultaneously, (S)-tert-butyl 1-(chloromethyl)-5-hydroxy-1H-benzo[e]indole-3(2H)-carboxylate (14) (97 mg, 0.29 mmol) was dissolved in anhydrous DMF (3 mL) at 0° C under nitrogen. A solution of bis(4-nitrophenyl) carbonate (106 mg, 0.35 mmol) and DMAP (43 mg, 0.35 mmol) in anhydrous DMF (1 mL), and pyridine (28 μL, 0.35 mol) were added sequentially. The reaction mixture was stirred at 0° C for 5 minutes and 30 minutes, respectively, at room temperature before it was concentrated in vacuo. The resulting residue, ted-butyl (S)-1-(chloromethyl)-5-(((4-nitrophenoxy)carbonyl)oxy)-1,2-dihydro-3H-benzo[e]indole-3-carboxylate (15) was dissolved in anhydrous DMF (6 mL) at 0° C under nitrogen before crude (2S,3S,4S,5R,6S)-2-(methoxycarbonyl)-6-(4-(((methyl(2-(methylamino)ethyl)carbamoyl)oxy)methyl)phenoxy)tetrahydro-2H-pyran-3,4,5-triyl triacetate (13) (0.53 mmol) in anhydrous DMF (0.5 mL) was added and the reaction stirred for 30 minutes at 0 °C. Upon completion, the reaction was concentrated under reduced pressure and purified by flash chromatography (silica gel; 0-100% petrol in ethyl acetate) to furnish (2S,3R,4S,5S,6S)-2-(4-((((2-(((((S)-3-(tert-butoxycarbonyl)-1-(chloromethyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (16) as a pale-yellow oil (233 mg, 88%). ESI-LCMS: m/z=936.0 [M+Na]⁺. calculated for C₄₄H₅₂ClN₃O₁₆Na=936.3; 89% purity by LC (A254 nm).

Trifluoroacetic acid (0.5 mL) was added dropwise to a solution of (S)-tert-butyl 1-(chloromethyl)-5-hydroxy-1H-benzo[e]indole-3(2H)-carboxylate (14) (137 mg, 0.411 mmol) in anhydrous dichloromethane (1.5 mL) at 0° C under nitrogen. The reaction mixture was stirred for 1 h at 0° C before being concentrated in vacuo. The resulting residue, (S)-1-(chloromethyl)-2,3-dihydro-1H-benzo[e]indol-5-ol (18) was dissolved in anhydrous DMF (1.2 mL) before pyridine (0.33 mL, 4.11 mmol), DMAP (5 mg, 41 μmol) in anhydrous DMF (0.12 mL), and glutaric anhydride (47 mg, 0.41 mmol) in anhydrous DMF (0.44 mL) were added at 0° C under nitrogen. After 10 minutes stirring at 0° C, the reaction was allowed to warm to room temperature and stirred for a further 2.5 h. The reaction mixture was concentrated under reduced pressure and purified by flash chromatography (silica gel; 0-50% methanol in dichloromethane) giving (S)-5-(1-(chloromethyl)-5-hydroxy-1,2-dihydro-3H-benzo[e]indol-3-yl)-5-oxopentanoic acid (19) as a brown solid (119 mg, 82% yield) ESI-LCMS: m/z=347.9 [M+H]⁺. calculated for C₁₈H₁₉ClNO₄=348.0; m/z=369.9 [M+Na]⁺. calculated for C₁₈H₁₈ClNO₄Na=370.1; 72% purity by LC (A254 nm).

16 (62 mg, 67 μmol) was dissolved in 4 N hydrochloric acid in 1,4-dioxane (0.5 mL) at 0 °C. The reaction mixture was stirred for 2.5 h at 0° C until completion before being concentrated under reduced pressure. The resulting residue, (2S,3R,4S,5S,6S)-2-(4-((((2-(((((S)-1-(chloromethyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (17) and (S)-5-(1-(chloromethyl)-5-hydroxy-1,2-dihydro-3H-benzo[e]indol-3-yl)-5-oxopentanoic acid (19) (15 mg, 43 μmol) were combined and dissolved in anhydrous DMF (1.5 mL) at 0° C under nitrogen before pyridine (5 μL, 62 μmol) and HATU (36 mg, 94 μmol) were added. The reaction was stirred overnight at room temperature, concentrated under reduced pressure and partially purified by flash chromatography (silica gel; 0-20% methanol in dichloromethane) to give (2S,3R,4S,5S,6S)-2-(4-((((2-(((((S)-1-(chloromethyl)-3-(5-((S)-1-(chloromethyl)-5-hydroxy-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (20) as a pale-yellow residue (30 mg, 61% yield). ESI-LCMS: m/z=1143.0 [M+H]⁺. calculated for C₅₇H₆₁Cl₂N₄O₁₇=1143.3; m/z=1165.0 [M+Na]⁺. calculated for C₅₇H₅₀Cl₂N₄O₁₇Na=1165.3; 52% purity by LC (A254 nm).

Lithium 3-(6-methyl-4-vinylpyridin-2-yl)propanoate (21) (50 g, 253.6 mmol) and EDCl (95.7 g, 500.6 mmol) were added sequentially to a solution of tert-butyl 1-amino-3,6,9,12,15,18,21,24-octaoxaheptacosan-27-oate (22) (124.8 g, 250.3 mmol) in DMF (1.5 L) with stirring. The solution was cooled to 5° C before DIPEA (175 mL, 1005 mmol) was added dropwise over 30 minutes. The reaction was stirred at room temperature for 12 h before being split in two with each 750 mL volume diluted with ethyl acetate (1 L) and washed with 5% aqueous lithium chloride (1 L). The aqueous layers were extracted twice with ethyl acetate (1 L), the organic layers were combined, dried over anhydrous sodium sulfate and concentrated under reduced pressure. The crude product was purified by flash chromatography (silica gel; 3% methanol in dichloromethane) to afford tert-butyl 1-(6-methyl-4-vinylpyridin-2-yl)-3-oxo-7,10,13,16,19,22,25,28-octaoxa-4-azahentriacontan-31-oate (23) as an oil (113.5 g, 68% yield). ¹H NMR (CDCl₃, 400 MHz) δ (ppm): 6.98 (1H, s), 6.96 (1H, s), 6.78 (1H, broad t), 6.58 (1H, dd, J=10.8 Hz and 17.6 Hz), 5.91 (1H, d, J=17.6 Hz), 5.42 (1H, d, J=10.8 Hz), 3.69 (2H, t, J=6.56 Hz), 3.62 (26H, m), 3.56 (2H, m), 3.48 (2H, t, J=4.9 Hz), 3.42 (2H, m, J=5 Hz), 3.07 (2H, t, 7.4 Hz), 2.62 (2H, t, J=7.4 Hz), 2.50 (3H, s,), 2.49 (2H, m), 2.19 (2H, broad t, J=9.2 Hz), 1.43 (9H, s). ESI-LCMS: m/z=671.4 [M+H]⁺. calculated for C₃₄H₅₉N₂O₁₁=671.4.

4 N Hydrochloric acid in 1,4-dioxane (300 mL, 1200 mmol) was added to a solution of 23 (113.5 g, 169 mmol) dissolved in 1,4-dioxane (200 mL). The solution was stirred for 8 h at room temperature and concentrated under reduced pressure to give 1-(6-methyl-4-vinylpyridin-2-yl)-3-oxo-7,10,13,16,19,22,25,28-octaoxa-4-azahentriacontan-31-oic acid hydrochloride (24) as an oily solid (100 g, 96% yield). ¹H NMR (CDCl₃, 400 MHz) δ (ppm): 7.95 (1H, broad s), 7.65 (1H, d, J=3.7 Hz), 7.36 (1H, s), 6.73 (1H, dd, J=10.8 Hz and 17.6 Hz), 6.27 (1H, d, J=17.6 Hz), 5.88 (1H, d, J=10.8 Hz), 3.76 (2H, m), 2.68 (3H, s), 3.64 (30H, broad m), 3.54 (2H, t, J=5.2 Hz), 3.50 (2H, t, J=7.5 Hz), 3.42 (2H, t, J=4.9 Hz), 2.98 (2H, t, J=7.5 Hz), 2.92 (3H, s), 2.59 (2H, t, J=6.0 Hz). ESI-LCMS: m/z=615.5 [M+H]⁺. calculated for C₃₀H₅₁N₂O₁₁=615.34.

N-Hydroxysuccinamide (293 mg, 2.54 mmol), N-(3-dimethylaminopropyl)-N′-ethylcarbodiimide hydrochloride (487 mg, 2.54 mmol) and DIPEA (890 μL, 5.10 mmol) were added to a solution of 24 (1.10 g, 1.79 mmol) in dichloromethane (30 mL). The resulting mixture was stirred at room temperature under nitrogen overnight and followed by LCMS analysis. The reaction mixture was diluted with dichloromethane (75 mL) and was washed with water (30 mL). The aqueous layer was further extracted with dichloromethane and the combined organic layers were dried over sodium sulfate, filtered and concentrated under reduced pressure in order to afford 2,5-dioxopyrrolidin-1-yl 1-(6-methyl-4-vinylpyridin-2-yl)-3-oxo-7,10,13,16,19,22,25,28-octaoxa-4-azahentriacontan-31-oate (25), as a yellow viscous oil (1.10 g, 86% yield). ¹H NMR (CDCl₃, 400 MHz) δ (ppm): 6.98 (1H, s), 6.97 (1H, s), 6.71 (1H, broad t, J=6.0 Hz), 6.61 (1H, dd, J=17.6 and 10.9 Hz), 5.93 (1H, d, J=17.6 Hz), 5.43 (1H, d, J=10.9), 3.84 (2H, t, J=6.5 Hz), 3.61 (26H, broad m), 3.49 (2H, t, J=5.1 Hz), 3.42 (2H, t, J=5.1 Hz), 3.25 (1H, dt, J=7.6 Hz), 3.06 (2H, t, J=7.4 Hz), 2.90 (2H, t, J=6.5 Hz), 2.83 (4H, broad d, J=2.7 Hz), 2.63 (2H, t, J=7.4 Hz), 2.51 (3H, s), 2.23 (1H, t, J=7.2 Hz), 2.22 (2H, s), 1.73 (5H, broad m), 1.23 (1H, t, J=7.2 Hz), 1.03 (3H, broad s). ESI-LCMS: m/z=712.8 [M+H]⁺. calculated for C₃₄H₅₄N₃O₁₃=712.4.

A mixture of 2,5-dioxopyrrolidin-1-yl 1-(6-methyl-4-vinylpyridin-2-yl)-3-oxo-7,10,13,16,19,22,25,28-octaoxa-4-azahentriacontan-31-oate (25) (900 mg, 1.26 mmol), 4-((S)-2-((S)-2-amino-3-methylbutanamido)-5-ureidopentanamido)benzyl tert-butyl ethane-1,2-diylbis(methylcarbamate) (26) (900 mg, 1.52 mmol) and DIPEA (488 mg, 3.78 mmol) in DMF (10 mL) was stirred under nitrogen for 2 h at room temperature. Solvent was removed in vacuo and the crude material was purified by flash chromatography (silica gel; 0-10% methanol in dichloromethane) to afford tert-butyl (4-((2S,5S)-5-isopropyl-37-(6-methyl-4-vinylpyridin-2-yl)-4,7,35-trioxo-2-(3-ureidopropyl)-10,13,16,19,22,25,28,31-octaoxa-3,6,34-triazaheptatriacontanamido)benzyl) ethane-1,2-diylbis(methylcarbamate) (27) as a yellow semi-solid (800 mg, 53%). ¹H NMR (CDCl₃, 400 MHz) δ (ppm): 9.16 (1H, s), 7.67 (2H, d, J=5.6 Hz), 7.62 (1H, d, J=7.5 Hz), 7.41 (1H, d, J=8.4 Hz), 7.24 (1H, broad t, J=5.7 Hz), 6.98 (2H, d, J=9.0 Hz), 6.75 (1H, m), 6.58 (1H, dd, J=10.8 Hz and 17.6 Hz), 5.89 (2H, broad overlapping singlets), 5.41 (1H, d, J=11.3 Hz), 5.17 (1H, broad s), 5.03 (1H, broad s), 4.63 (1H, t, J=8.5 Hz), 4.29 (1H, t, J=7.7 Hz), 3.79 (1H, m), 3.73 (1H, s), 3.60 (36H, broad m), 3.49 (3H, broad m), 3.40 (3H, broad m), 3.33 (5H, broad m), 3.21 (2H, broad m, 3.05 (2H, t, J=7.6 Hz), 2.91 (4H, broad m), 2.86 (2H, broad overlapping singlets), 2.75 (2H, broad overlapping singles), 2.62 (3H, t, J=7.7 Hz), 2.49 (3H, s), 2.22 (1H, q, J=6.4 Hz), 1.99 (1H, broad m), 1.72 (1H, broad m), 1.55 (2H, m), 1.42 (9H, s), 0.96 (8H, t, J=6.7 Hz). ESI-LCMS: m/z=596.2 [M+2H]²⁺. calculated for C₅₈H₉₇N₉O₁₇=1191.7; m/z=606.90 [M+H+Na]²⁺. calculated for C₅₈H₉₆NO₁₇Na=1213.7.

27 (55 mg, 46 μmol) was dissolved in trifluoroacetic acid at 0 °C. The solution was stirred at 0° C for 15 minutes and concentrated under reduced pressure to afford crude 4-((2S,5S)-5-isopropyl-37-(6-methyl-4-vinylpyridin-2-yl)-4,7,35-trioxo-2-(3-ureidopropyl)-10,13,16,19,22,25,28,31-octaoxa-3,6,34-triazaheptatriacontanamido)benzyl methyl(2-(methylamino)ethyl)carbamate (28). Di(N-succinimidyl) carbonate (4 mg, 16 μmol) and triethylamine (2.2 μL, 16 μmol) were added to (2S,3R,4S,5S,6S)-2-(4-((((2-(((((S)-1-(chloromethyl)-3-(5-((S)-1-(chloromethyl)-5-hydroxy-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (20) (13 mg, 10 μmol) in anhydrous dichloromethane at 0° C under nitrogen. The reaction was stirred at 0° C for 15 minutes then at room temperature for a further 30 minutes before crude 28 (19 μmol) in DMF (1 mL) and DMAP (2 mg, 19 μmol) were added under nitrogen. The reaction was stirred at 0° C for 1 h and then at room temperature overnight. After stirring overnight, more TEA (3 μL, 22 μmol) was added and the reaction continued for a further 6 h before it was concentrated under reduced pressure and purified by flash chromatography (silica gel; 3-100% methanol in dichloromethane) to give (2S,3R,4S,5S,6S)-2-(4-((((2-(((((S)-1-(chloromethyl)-3-(5-((S)-1-(chloromethyl)-5-(((2-((((4-((2S,5S)-5-isopropyl-37-(6-methyl-4-vinylpyridin-2-yl)-4,7,35-trioxo-2-(3-ureidopropyl)-10,13,16,19,22,25,28,31-octaoxa-3,6,34-triazaheptatriacontanamido)benzyl)oxy)carbonyl)(methyl)amino)ethyl)(methyl)carbamoyl)oxy)-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (30) (20 mg, 78%) as a residue. ESI-LCMS: m/z=1130.7 [M+2H]². calculated for C₁₁₁H₁₄₇Cl₂N₁₃O₃₃=2260.0; m/z=754.2 [M+3H]³⁺. calculated for C₁₁₁H₁₄₈Cl₂N₁₃O₃₃=2261.0; 81% purity by LC (A254 nm).

A solution of lithium hydroxide monohydrate (1 mg, 22 μmol) in water (0.5 mL) was added to 30 (5 mg, 2.2 μmol) in methanol (0.5 mL) at 0 °C. The reaction was stirred at 0° C for 1.5 h until complete. The methanol was removed under reduced pressure before the remaining aqueous solution was cooled to 0° C and neutralised with 0.1 M hydrochloric acid. The neutralised solution was concentrated in vacuo to give 6 mg of crude product. The reaction was repeated using lithium hydroxide monohydrate (2 mg, 44 μmol) in water (1 mL) and 30 (10 mg, 4.4 μmol) in methanol (1 mL) to give another 8 mg of crude product. The crude materials were combined and purified by reverse-phase semi-preparative HPLC to afford (2S,3S,4S,5R,6S)-6-(4-((((2-(((((S)-1-(chloromethyl)-3-(5-((S)-1-(chloromethyl)-5-(((2-((((4-((2S,5S)-5-isopropyl-37-(6-methyl-4-vinylpyridin-2-yl)-4,7,35-trioxo-2-(3-ureidopropyl)-10,13,16,19,22,25,28,31-octaoxa-3,6,34-triazaheptatriacontanamido)benzyl)oxy)carbonyl)(methyl)amino)ethyl)(methyl)carbamoyl)oxy)-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)phenoxy)-3,4,5-trihydroxytetrahydro-2H-pyran-2-carboxylic acid (1) as a white solid (2 mg, 14% yield). ESI-LCMS: m/z=1060.5 [M+2H]²⁺. calculated for C₁₀₄H₁₃₉Cl₂N₁₃O₃₀=2119.9; m/z=1070.5 [M+H+Na]²⁺. calculated for C₁₀₄H₁₃₈Cl₂N₁₃O₃₀Na=2141.9. HPLC: >99% Purity (A260 nm).

### Preparation of Compound 2

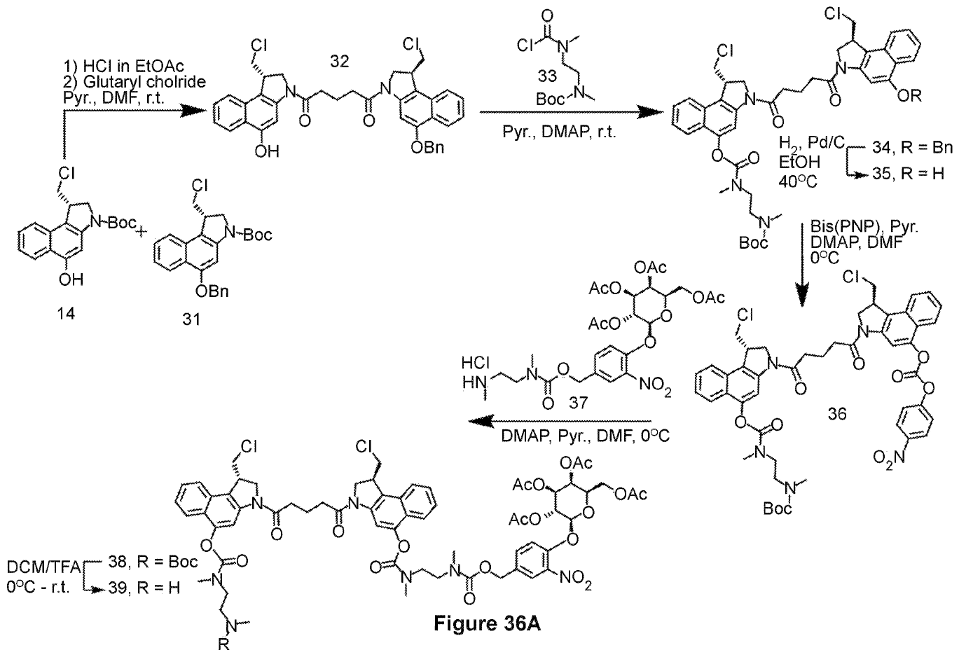

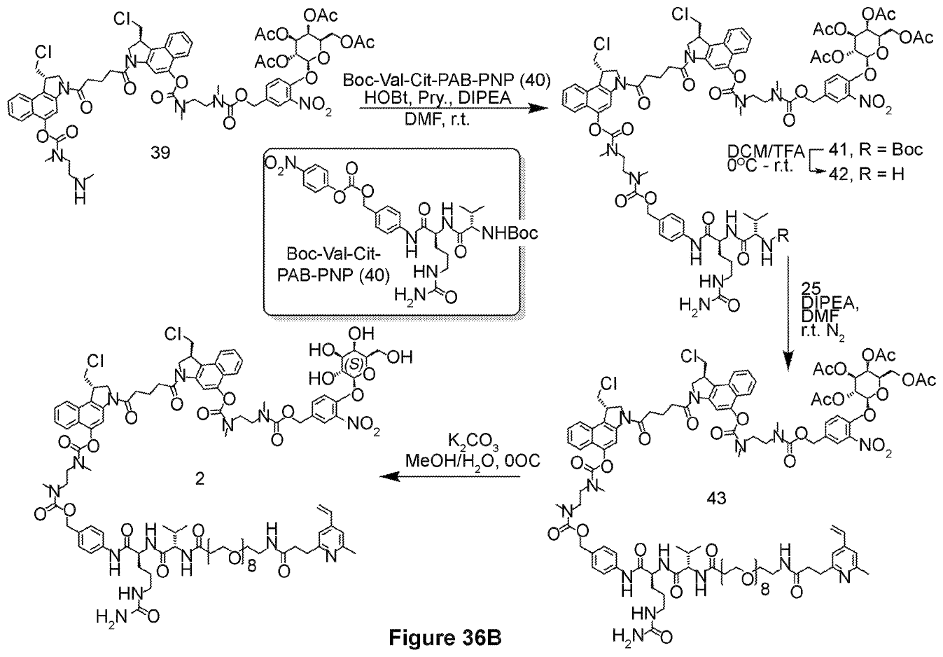

(S)-1-(chloromethyl)-3-(5-((S)-1-(chloromethyl)-5-(((2-((((4-((2S,5S)-5-isopropyl-37-(6-methyl-4-vinylpyridin-2-yl)-4,7,35-trioxo-2-(3-ureidopropyl)-10,13,16,19,22,25,28,31-octaoxa-3,6,34-triazaheptatriacontanamido)benzyl)oxy)carbonyl)(methyl)amino)ethyl)(methyl)carbamoyl)oxy)-1,2-dihydro-3H-benzo[e]indol-3-yl)-5-oxopentanoyl)-2,3-dihydro-1H-benzo[e]indol-5-yl (3-nitro-4-(((2S,3R,4S,5R,6R)-3,4,5-trihydroxy-6-(hydroxymethyl)tetrahydro-2H-pyran-2-yl)oxy)benzyl) ethane-1,2-diylbis(methylcarbamate) (2) was prepared according to the schemes shown in FIGS. 36A and 36B.

4 N Hydrochloric acid in ethyl acetate (20 mL) was added to (S)-tert-butyl 1-(chloromethyl)-5-hydroxy-1H-benzo[e]indole-3(2H)-carboxylate (14) (200 mg, 0.60 mmol) and (S)-tert-butyl 5-(benzyloxy) 1-(chloromethyl)-1H-benzo[e]indole-3(2H)-carboxylate (31) (254 mg, 0.60 mmol) at room temperature. After stirring for 3 h, the reaction mixture was concentrated under reduced pressure and dried under high vacuum for 1 h. The residue was dissolved in DMF (5 mL), and pyridine (144 μL, 1.79 mmol) was added at 0° C followed by glutaryl chloride (76 μL, 0.60 mmol). The resulting mixture was allowed to warm to room temperature and was stirred for 20 minutes. Upon completion, the reaction mixture was quenched with ice cold 1 N hydrochloric acid (10 mL) and extracted with ethyl acetate (4×20 mL). The combined organic layers were washed with aqueous sodium bicarbonate, brine, dried over magnesium sulphate and concentrated under reduced pressure. The crude product was partially purified by flash chromatography (silica gel; 35-65% ethyl acetate in petroleum ether) to furnish a brown solid (70 mg) of 1-((S)-5-(benzyloxy)-1-(chloromethyl)-1,2-dihydrobenzo[e]indol-3-yl)-5-((S)-1-(chloromethyl)-1,2-dihydro-5-hydroxybenzo[e]indol-3-yl)pentane-1,5-dione (32) as mixture with 1-((S)-1-(chloromethyl)-1,2-dihydro-5-hydroxybenzo[e]indol-3-yl)-5-((S)-1-(chloromethyl)-1,2-dihydro-5-hydroxybenzo[e]indol-3-yl)pentane-1,5-dione. ESI-HRMS (32): m/z=653.1968 [M+H]⁺. calculated for C₃₈H₃₅Cl₂N₂O₄=653.1944.

Partially purified 32 (70 mg, 0.107 mmol) and tert-butyl (2-((chlorocarbonyl)(methyl)amino)ethyl)(methyl)carbamate (33) (54 mg, 0.215 mmol) were dissolved in dry pyridine (2 mL) and DMAP (2.6 mg, 21.4 μmol, 20 mol %) was added. The reaction mixture was stirred overnight at room temperature, diluted with 1N hydrochloric acid (15 mL) and extracted with ethyl acetate (4×20 mL). The combined organic layers were washed with aqueous sodium bicarbonate, brine, dried over magnesium sulphate and concentrated under reduced pressure. The crude product was purified by flash column chromatography (silica gel; 50% ethyl acetate in petroleum ether) to furnish (S)-3-(5-((S)-5-(benzyloxy)-1-(chloromethyl)-1H-benzo[e]indol-3(2H)-yl)-5-oxopentanoyl)-1-(chloromethyl)-2,3-dihydro-1H-benzo[e]indol-5-yl tert-butyl ethane-1,2-diylbis(methylcarbamate) (34) as a brown solid (70 mg, 75% yield). ¹H NMR (500 MHz, DMSO-d₆) δ (ppm): 1.34-1.44 (m, 9H, Boc), 1.96-2.01 (m, 2H, 2-H), 2.62-2.71 (m, 4H, 1-H, 3-H), 2.72-3.19 (m, 6H, 2×CH₃—N), 3.40-3.47 (m, 2H, CH₂—N), 3.48-3.60 (m, 1H, CH₂—N), 3.64-3.68 (m, 1H, CH₂—N), 3.85 (t, J=10 Hz, 1H, 10′-H_{b}), 3.93 (dd, J=11.1, 7.4 Hz, 1H, 10′-Hₐ), 3.99-4.64 (m, 2H, 10′-H_{b}, 10′-Hₐ), 4.21-4.44 (m, 6H, 1′-H, 2′-H_{b}, 2′-Hₐ), 5.27 (s, 2H, Bn-H), 7.34-7.58 (m, 10H, 5×PhH, 7′-H, 8′-H, 9′-H), 7.86 (d, J=7.4 Hz, 1H, 9′-H), 7.96 (d, J=8.3 Hz, 1H, 6′-H), 8.18 (d, J=8.5 Hz, 1H, 6′-H), 8.20 (s, 1H, 4′-H), 8.24 (s, 1H, 4′-H). ¹³C NMR (125 MHz, DMSO-d₆) δ (ppm): 19.05 (C-2), 28.02, 28.12 (C-Boc), 31.21, 34.33, 34.42 (CH₃, C-1 or C-3), 40.69 (C-1′), 40.84 (C-1′), 44.50 (C—CH₂), 47.76 (C-10′), 52.61 (C-2′), 52.68 (C-2′), 69.60 (C-Bn), 78.69 (Boc), 97.81 (C-4′), 110.11 (C-4′), 115.46 (C-9b′), 121.04 (C-5a′), 122.16 (C-6′), 122.40 (C-9′), 122.68 (C-6′), 122.81 (C-9b′), 123.21 (C-4′), 123.60 (C-9b′), 127.53 (2×Ph-Cₒ), 127.63 (C-9′), 127.96 (Ph-Cₚ), 128.57 (Ph-Cₘ), 129.60 (C-9a′), 129.76 (C-9a′), 136.86 (C-1″), 141.22 (C-9a′), 142.06 (C-9a′), 147.55 (C-3a′), 153.88 (CO), 154.61 (C-5′), 171.02 (CO). ESI-HRMS: m/z=889.3105 [M+Na]⁺. calculated for C₄₈H₅₂Cl₂N₄O₇Na=889.3089. Optical Rotation: [α]D=−20.5 (c=0.30, MeOH, 21.4° C). IR (ATR): \tilde{v} (cm⁻¹)=2976, 1715, 1656, 1460, 1399, 1036, 754.

A mixture of 34 (20 mg, 0.023 mmol) and Pd/C (10 wt. % loading, 10 mg, 9.4 μmol) in ethanol (2 mL) was evacuated, flushed with argon, again evacuated and then flushed with hydrogen using a balloon. The reaction mixture was heated to 40° C with stirring. After completion of the reaction, the mixture was filtered over celite and the residue washed with ethyl acetate (3×5 mL). The combined filtrate was concentrated under reduced pressure and the crude tert-butyl ((S)-1-(chloromethyl)-3-(5-((S)-1-(chloromethyl)-5-hydroxy-1,2-dihydro-3H-benzo[e]indol-3-yl)-5-oxopentanoyl)-2,3-dihydro-1H-benzo[e]indol-5-yl) ethane-1,2-diylbis(methylcarbamate) (35) was used without further purification. ESI-HRMS: m/z=799.2636 [M+Na]⁺. calculated for C₄₁H₄₆C₂N₄O₇Na=799.2623.

To a solution of crude 35 (49.8 mg, 64 μmol) in DMF (0.5 mL), bis(4-nitro-phenyl)carbamate (38.9 mg, 128 μmol), pyridine (10 mg, 128 μmol, 10 μL) and DMAP (15 mg, 124 μmol) were added at 0 °C. After 10 minutes, (2R,3S,4S,5R,6S)-2-(acetoxymethyl)-6-(4-(((methyl(2-(methylamino)ethyl)carbamoyl)oxy)methyl)-2-nitrophenoxy)tetrahydro-2H-pyran-3,4,5-triyl triacetate hydrochloride salt (37) (92 mg, 142 μmol) was added and stirring continued for 30 minutes at 0 °C. Upon completion, the mixture was concentrated under reduced pressure and purified by preparative TLC (petroleum ether/ethyl acetate=1:9) to furnish (2R,3S,4S,5R,6S)-2-(acetoxymethyl)-6-(4-((((2-(((((S)-3-(5-((S)-5-(((2-((tert butoxycarbonyl)(methyl)amino)ethyl)(methyl)carbamoyl)oxy)-1-(chloromethyl)-1H-benzo[e]indol-3(2H)-yl)-5-oxopentanoyl)-1-(chloromethyl)-2,3-dihydro-1H-benzo[e]indol-5-yl)oxy)carbonyl)(methyl)amino)ethyl)(methyl)carbamoyl)oxy)methyl)-2-nitrophenoxy)tetrahydro-2H-pyran-3,4,5-triyl triacetate (38) as a white solid (23 mg, 25% yield). ¹H NMR (500 MHz, DMSO-d₆, 80° C) δ (ppm): 1.38-1.43 (m, 9H, Boc), 1.98-2.14 (m, 14H, 3×OAc, 2-H), 2.61-2.77 (m, 4H, 1-H, 3-H), 2.84-3.0 (m, 9H, 3×CH₃), 3.19-3.21 (m, 3H, CH₃), 3.44-4.41 (m, 19H, 10′-Hb, 10′-Hₐ, 4×CH₂, 1′-H, 6′″-H, 5′″-H, 2′-H_{b}, 2′-Hₐ), 5.06-5.47 (m, 6H, 1′″-H, 2′″-H, 3′″-H, 4′″-H, Bn-H), 7.31-8.22 (m, 13H, Ar). ¹³C NMR (125 MHz, DMSO-d₆, 80° C) δ (ppm): 18.90 (C-2), 19.86, 19.87, 19.95, 20.01 (OAc), 27.81 (Boc), 34.02, 34.04, 34.56 (C-1, C-3, N—CH₃), 40.63 (C-1′), 46.49 (CH₂), 47.23 (C-10′), 47.25 (C-10′), 52.47 (C-2′), 60.92 (C-6′″), 64.56 (C-Bn), 66.95, 67.70, 69.7 (C-2′″, C-3′″, C-4′″), 70.63 (C-5′″), 98.64 (C-1′″), 99.00 (C-4′), 109.69 (C-4′), 117.86 (C-9b′), 120.76, 121.91, 122.06, 122.75, 123.44, 123.76, 124.14, 124.63, 127.07, 127.09, 128.33, 128.42, 129.32, 129.34, 129.36, 129.38, 131.13, 131.21, 131.62, 131.64, 132.24, 132.42, 132.88, 133.05, 140.15, 140.90, 147.37, 147.60, 153.55, 154.70, 168.43 (OAc), 169.03 (OAc), 169.40 (OAc), 169.49 (OAc), 170.58 (CO), 170.64 (CO). ESI-HRMS: m/z=1438.4547 [M+Na]⁺. calculated for C₆₈H₇₉Cl₂N₇O₂₂Na=1438.4521. Optical Rotation: [α]D=−78.0 (c=0.59, MeOH, 23.3° C). IR (ATR): \tilde{v} (cm⁻¹)=2944, 2831, 1723, 1656, 1402, 1107, 1020, 754.

Trifluoracetic acid in dichloromethane (0.2 mL, 1:1) was added to 38 (20 mg, 14 μmol) at 0 °C. The reaction mixture was allowed to warm to room temperature and stirred for 1 h. The solvent was evaporated under reduced pressure. The resulting residue (39) was dissolved in DMF (0.1 mL) and Boc-Val-Cit-PAB-PNP (40) (22 mg, 34 μmol), 1-hydroxybenzotriazole (4 mg, 28 μmol), pyridine (1 mg, 14 μmol, 1 μL) and DIPEA (4 mg, 28 μmol, 5 μL) were added sequentially and the reaction mixture was stirred overnight at room temperature. Upon completion, the mixture was concentrated under reduced pressure and purified by preparative TLC (ethyl acetate/methanol=9.5:0.5) to afford (2R,3S,4S,5R,6S)-2-(acetoxymethyl)-6-(4-((((2-(((((S)-3-(5-((S)-5-(((2-((((4-((S)-2-((S)-2-((tert-butoxycarbonyl)amino)-3-methylbutanamido)-5-ureidopentanamido)benzyl)oxy)carbonyl)(methyl)amino)ethyl)(methyl)carbamoyl)oxy)-1-(chloromethyl)-1,2-dihydro-3H-benzo[e]indol-3-yl)-5-oxopentanoyl)-1-(chloromethyl)-2,3-dihydro-1H-benzo[e]indol-5-yl)oxy)carbonyl)(methyl)amino)ethyl)(methyl)carbamoyl)oxy)methyl)-2-nitrophenoxy)tetrahydro-2H-pyran-3,4,5-triyl triacetate (41) as a white solid (5 mg, 20%). ¹H NMR (500 MHz, DMSO-d₆, 80° C) δ (ppm): 0.86 (ddd, J=17.8, 6.8, 4.2 Hz, 10H, Isopropyl-Peptide), 1.12-1.52 (m, 27H, Boc, H-Peptide), 1.58-1.69 (m, 1H, H-Peptide), 1.69-1.73 (m, 1H, H-Peptide), 1.93-2.13 (m, 16H, 4×OAc, 2-H, H-Peptide), 2.60-2.67 (m, 2H, 1-H or 3-H), 2.69-2.76 (m, 2H, 3-H or 1-H), 2.90-3.04 (m, 13, N—CH₃, H-Peptide, under H₂O peak), 3.36-3.78 (m, 8H, N—CH₂), 3.83-3.93 (m, 3H, 2×10′-H_{b}, H-Peptide), 4.04 (ddd, J=11.0, 3.3, 1.2 Hz, 2H, 10′-Hₐ), 4.10-4.13 (m, 2H, 6″-Hₐ, 6″-H_{b}), 4.22-4.49 (m, 8H, 2×2′-H_{b}, 2×2′-Hₐ, 2×1′-H, 5″-H,H-Peptide), 5.04 (s, 2H, Bn), 5.13 (s, 2H, Bn), 5.23 (d, J=8.7 Hz, 4H, 2′″-H, 4′″-H, NH₂), 5.35 (d, J=4.4 Hz, 1H, 3′″-H), 5.50 (m, 1H, 1′″-H), 5.85 (t, J=5.8 Hz, 1H, NH), 6.12 (d, J=9.1 Hz, 0.5H, NH), 6.45 (s, 0.5H, NH), 7.13-8.38 (m, 17H, Ar—H), 9.82 (s, 1H, NH). ¹³C NMR (125 MHz, DMSO-d₆, 80° C) δ (ppm): 18.04, 18.06 (Isopropyl), 19.15 (Isopropyl), 19.22 (C-2), 20.15, 20.16, 20.24, 20.30 (OAc), 21.96, 25.07, 26.61, 28.20, 28.22, 28.54, 28.60 (Boc), 29.06, 29.68, 29.74, 30.42, 31.19 (Peptide), 34.35 (C-3 Or C-1), 34.86 (N—CH₃), 35.18 (N—CH₃), 38.81 (Peptide), 40.96 (C-1′), 46.82 (N—CH₂), 47.53 (C-10′), 52.81 (C-2′), 53.07 (Peptide), 61.23 (C-6′″), 62.74 (Peptide), 64.88 (Bn), 66.19 (Bn), 67.28 (C-3′″), 68.04 (C-2′″ or C-4′″), 70.11 (C-2′″ or C-4′″), 70.96 (C-5′″), 78.29 (Boc), 98.97 (C-1′″), 109.96 (C-4′), 118.21, 119.24, 119.30, 121.09, 122.26, 123.05, 124.10, 124.49, 126.89, 127.39, 128.25, 129.67, 137.43, 137.65, 138.47, 140.49, 147.72, 147.90, 158.87 (NCO), 168.73 (OAc), 169.32 (OAc), 169.69 (OAc), 169.78 (OAc), 170.28 (CO), 170.44 (CO), 170.91 (CO), 170.94 (CO), 171.33 (CO). ESI-HRMS: m/z=1843.6560 [M+Na]⁺. calculated for C₈₇H₁₀₆Cl₂N₁₂O₂₇Na=1843.6559. Optical Rotation: [α]_{D}=−17.2 (c=0.5, MeOH, 22.3° C). IR (ATR): \tilde{v} (cm⁻¹)=3323, 2834, 1650, 1543, 1012, 750.

A solution of 41 (25 mg, 13.7 μmol) in dichloromethane (1 mL) was treated with trifluoracetic acid (0.2 mL) at 0 °C. The reaction mixture was allowed to warm to room temperature and stirred for 2 h. Evaporation of the solvent under reduced pressure gave (2R,3S,4S,5R,6S)-2-(acetoxymethyl)-6-(4-((((2-(((((S)-3-(5-((S)-5-(((2-((((4-((S)-2-((S)-2-amino-3-methylbutanamido)-5-ureidopentanamido)benzyl)oxy)carbonyl)(methyl)amino)ethyl)(methyl)carbamoyl)oxy)-1-(chloromethyl)-1,2-dihydro-3H-benzo[e]indol-3-yl)-5-oxopentanoyl)-1-(chloromethyl)-2,3-dihydro-1H-benzo[e]indol-5-yl)oxy)carbonyl)(methyl)amino)ethyl)(methyl)carbamoyl)oxy)methyl)-2-nitrophenoxy)tetrahydro-2H-pyran-3,4,5-triyl triacetate (42) as a white or green solid in quantitative yield and was used without further purification. ¹H NMR (500 MHz, DMSO-d₆, 80° C) δ (ppm): 0.82-0.96 (m, 8H, Isopropyl-Peptide), 1.23-1.63 (m, 17H, H-Peptide), 1.78-1.76 (m, 1H, H-Peptide), 1.90-2.12 (m, 12H, 3×OAc, 2-H, H-Peptide), 2.10 (s, 3H, OAc), 2.56-2.59 (m, 4H, 1-H or 3-H), 2.61-2.91 (m, 13H, N—CH₃, H-Peptide, under H₂O peak), 3.50-3.67 (8H, N—CH₂), 3.86-3.90 (m, 2H, 2×10′-H_{b}), 3.99-4.02 (m, 2H, 10′-H_{b}), 4.08-4.42 (m, 9H, 6″-Hₐ, 6″-H_{b}, 2′-H_{b}, 2′-Hₐ, 1′-H, 5″-H), 5.01-5.43 (m, 8H, Bn, 2′″-H, 4′″-H, 3′″-H,1′″-H), 7.20-8.36 (m, 17H, Ar—H), 9.82 (s, 1H, NH). ESI-HRMS: m/z=1721.6192 [M+H]⁺. calculated for C₈₂H₉₉Cl₂N₁O₂₅=1721.6216.

To a solution of 42 (14 mg, 8.12 μmol) in anhydrous DMF (0.5 mL), 2,5-dioxopyrrolidin-1-yl 1-(6-methyl-4-vinylpyridin-2-yl)-3-oxo-7,10,13,16,19,22,25,28-octaoxa-4-azahentriacontan-31-oate (25) (13 mg, 18.3 μmol) in DMF (0.5 mL), and DIPEA (10.3 mg, 79.7 μmol, 14 μL) were added sequentially under nitrogen. The reaction was stirred at room temperature overnight before being concentrated under reduced pressure to give crude product. A repeat reaction was performed using a solution of 42 (3 mg, 1.74 μmol) in anhydrous DMF (0.15 mL) 25 (2 mg, 2.18 μmol) in DMF (0.15 mL), and DIPEA (2 mg, 13.93 μmol, 2.4 μL). The crude product from both reactions were combined and purified by flash chromatography (silica gel; 2.5-50% methanol in dichloromethane) to afford (2R,3S,4S,5R,6S)-2-(acetoxymethyl)-6-(4-((((2-(((((S)-1-(chloromethyl)-3-(5-((S)-1-(chloromethyl)-5-(((2-((((4-((2S,5R)-5-isopropyl-37-(6-methyl-4-vinylpyridin-2-yl)-4,7,35-trioxo-2-(3-ureidopropyl)-10,13,16,19,22,25,28,31-octaoxa-3,6,34-triazaheptatriacontanamido)benzyl)oxy)carbonyl)(methyl)amino)ethyl)(methyl)carbamoyl)oxy)-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)-2-nitrophenoxy)tetrahydro-2H-pyran-3,4,5-triyl triacetate (43) (12 mg, 52% yield). ESI-LCMS: m/z=1160.0 [M+2H]². calculated for C₁₁₂H₁₄₈Cl₂N₁₄O₃₅=2319.0; m/z=773.8 [M+3H]³⁺. calculated for C₁₁₂H₁₄₉Cl₂N₁₄O₃₅=2320.0; 80% purity by LC (A254 nm).

Potassium carbonate (6 mg, 43 μmol) in water (1 mL) was added to a solution of 43 (10 mg, 4.3 μmol) in methanol (1 mL). The reaction was stirred at room temperature for 5 h until complete. The methanol was removed under reduced pressure and the remaining aqueous solution neutralised with 0.1 N hydrochloric acid before being concentrated in vacuo. The crude product was dissolved in a minimal amount of water, applied to a C18 cartridge and eluted in acetonitrile to give (S)-1-(chloromethyl)-3-(5-((S)-1-(chloromethyl)-5-(((2-((((4-((2S,5S)-5-isopropyl-37-(6-methyl-4-vinylpyridin-2-yl)-4,7,35-trioxo-2-(3-ureidopropyl)-10,13,16,19,22,25,28,31-octaoxa-3,6,34-triazaheptatriacontanamido)benzyl)oxy)carbonyl)(methyl)amino)ethyl)(methyl)carbamoyl)oxy)-1,2-dihydro-3H-benzo[e]indol-3-yl)-5-oxopentanoyl)-2,3-dihydro-1H-benzo[e]indol-5-yl (3-nitro-4-(((2S,3R,4S,5R,6R)-3,4,5-trihydroxy-6-(hydroxymethyl)tetrahydro-2H-pyran-2-yl)oxy)benzyl) ethane-1,2-diylbis(methylcarbamate) (2) as a residue (8.6 mg, 93% yield). ESI-LCMS: m/z=1076.0 [M+2H]²⁺. calculated for C₁₀₄H₁₄₀Cl₂N₁₄O₃₁=2150.9; m/z=717.8 [M+3H]³⁺; calculated for C₁₀₄H₁₄₁Cl₂N₁₄O₃₁=2151.9; 80% purity by LC (A254 nm).

### Preparation of Compound 3

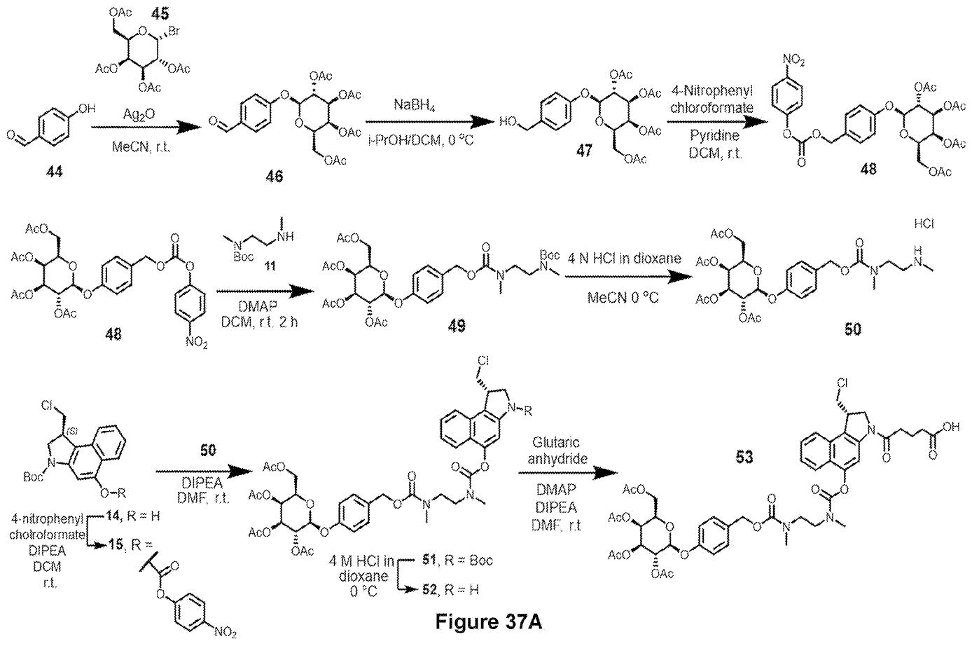

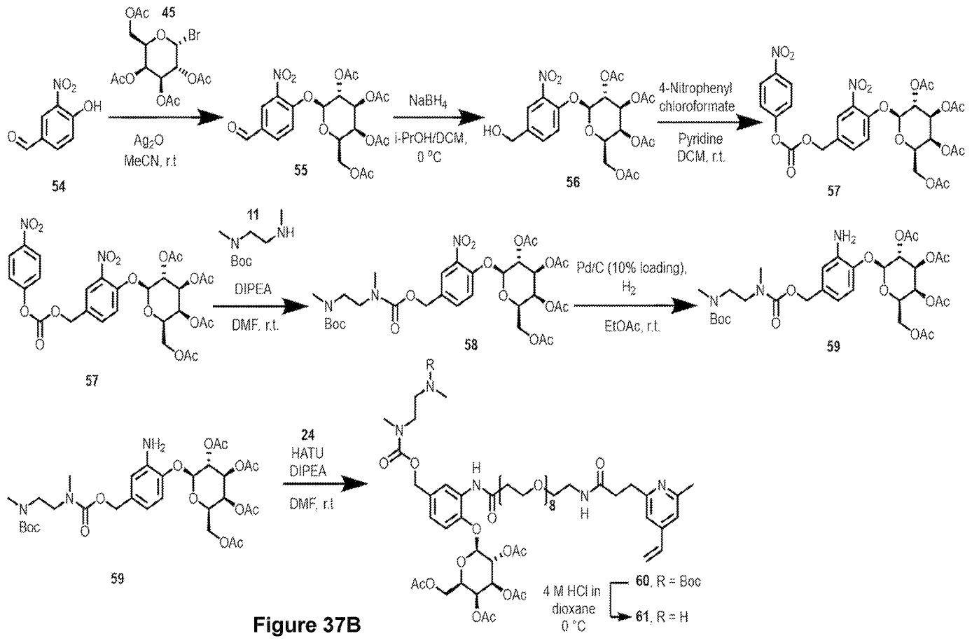

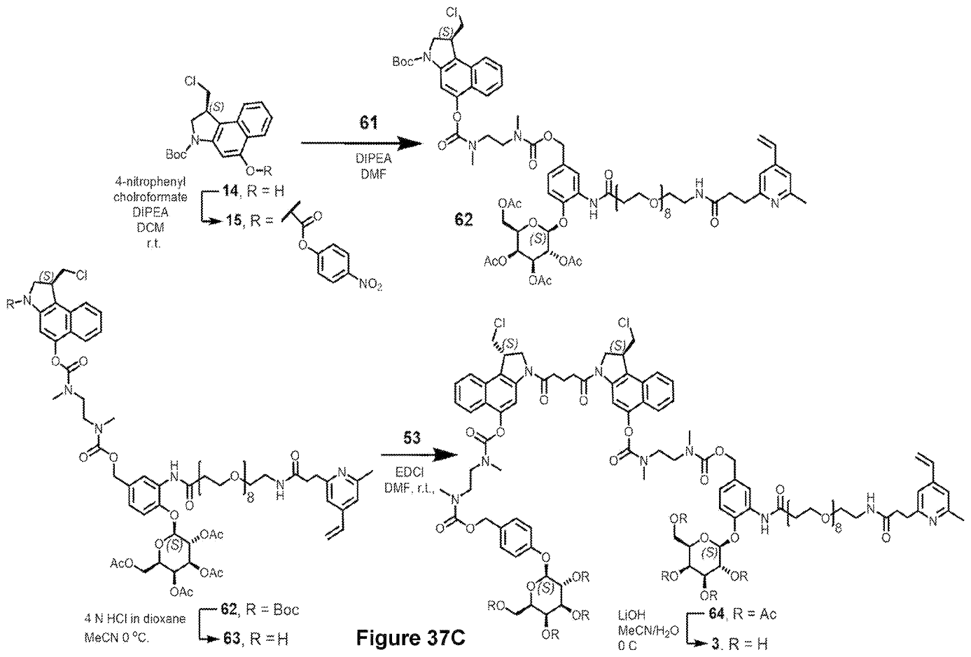

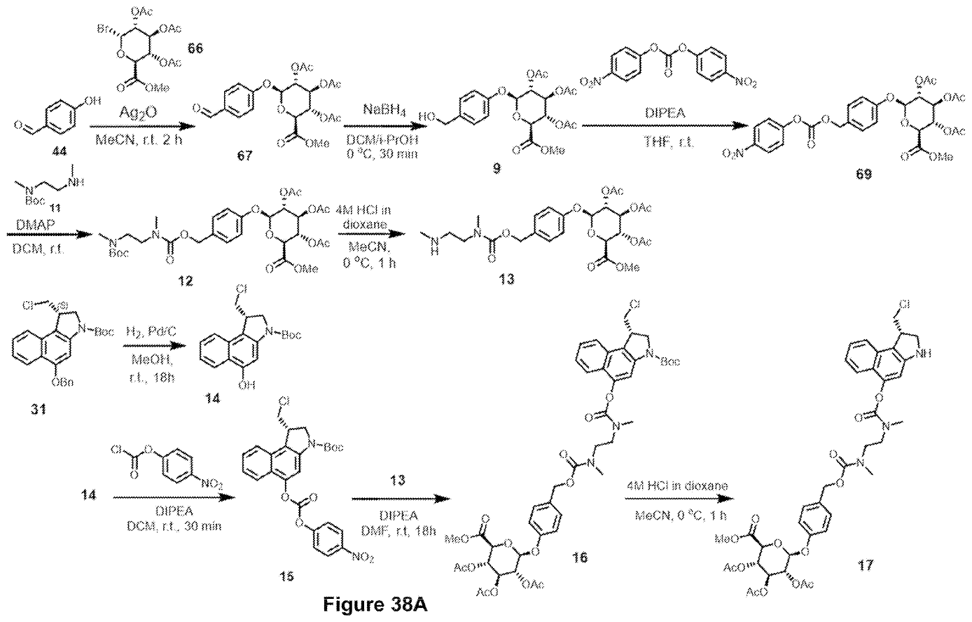

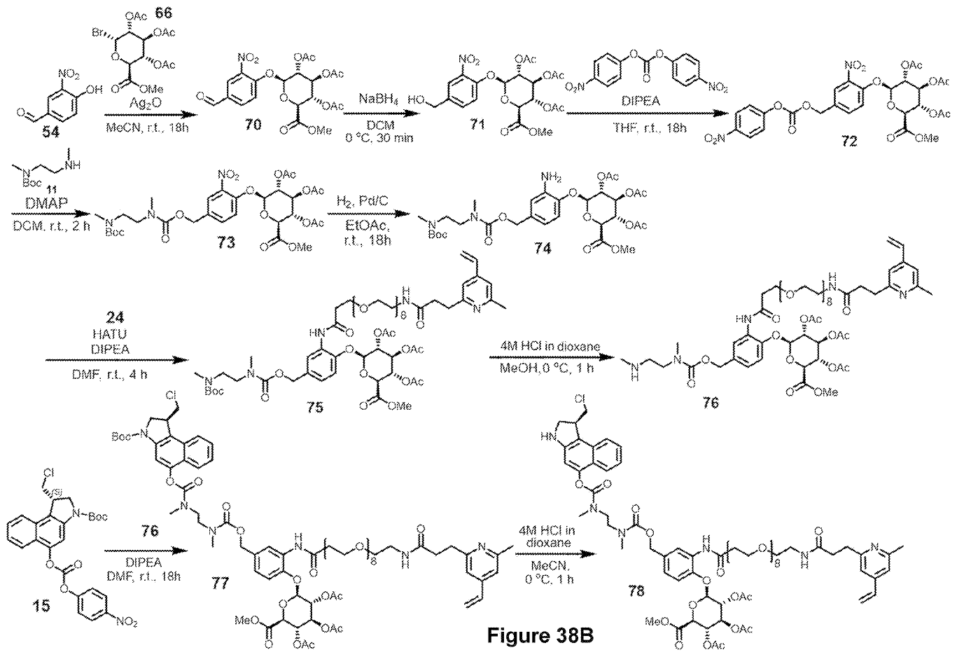

(S)-1-(chloromethyl)-3-(5-((S)-1-(chloromethyl)-5-((methyl(2-(methyl(((3-(1-(6-methyl-4-vinylpyridin-2-yl)-3-oxo-7,10,13,16,19,22,25,28-octaoxa-4-azahentriacontan-31-amido)-4-(((2S,3R,4S,5R,6R)-3,4,5-trihydroxy-6-(hydroxymethyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoyl)-2,3-dihydro-1H-benzo[e]indol-5-yl (4-(((2S,3R,4S,5R,6R)-3,4,5-trihydroxy-6-(hydroxymethyl)tetrahydro-2H-pyran-2-yl)oxy)benzyl) ethane-1,2-diylbis(methylcarbamate) (3) was prepared according to the schemes shown in FIGS. 37A to 37C.

To a solution of 4-hydroxybenzaldehyde (44) (5.0 g, 40.9 mmol) in acetonitrile (80 mL) was added (2R,3S,4S,5R,6R)-2-(acetoxymethyl)-6-bromotetrahydro-2H-pyran-3,4,5-triyl triacetate (45) (20.2 g, 49.13 mmol) and silver (1) oxide (18.98 g, 81.89 mmol). The mixture was stirred at room temperature overnight. The reaction mixture was filtrated, washed with dichloromethane (200 mL) and the filtrate was concentrated. The residue was purified by flash chromatography (silica gel; 25% ethyl acetate in petroleum ether) to afford (2R,3S,4S,5R,6S)-2-(acetoxymethyl)-6-(4-formylphenoxy)tetrahydro-2H-pyran-3,4,5-triyl triacetate (46) as a white solid (12.6 g, 68%). R_{f}=0.3 (silica gel; petroleum ether/ethyl acetate=2:1). ¹H NMR (400 MHz, DMSO-d₆) δ (ppm): 9.91 (s, 1H), 7.95-7.89 (m, 2H), 7.21-7.15 (m, 2H), 5.67 (d, J=7.4 Hz, 1H), 5.37 (dd, J=3.2, 1.2 Hz, 1H), 5.33-5.22 (m, 2H), 4.53-4.46 (m, 1H), 4.10 (d, J=6.4 Hz, 2H), 2.15 (s, 3H), 2.03 (s, 3H), 2.01 (s, 3H), 1.95 (s, 3H). ESI-LCMS: m/z=453.1 [M+H]⁺; calculated for C₂₁H₂₅O₁₁=453.1.

To a solution of 46 (11.6 g, 25.64 mmol) and silica gel (580 mg) in dichloromethane/isopropanol (4:1) at 0° C was slowly added sodium borohydride (1.07 g, 28.2 mmol). The mixture was stirred at 0° C for 1.5 h. The mixture was diluted with ethyl acetate (30 mL) and filtered. The filtrate was poured into water (100 mL) and extracted with dichloromethane (3×30 mL). The combined organic phases were washed with brine (40 mL), dried over sodium sulfate and concentrated to afford (2R,3S,4S,5R,6S)-2-(acetoxymethyl)-6-(4-(hydroxymethyl)phenoxy)tetrahydro-2H-pyran-3,4,5-triyl triacetate (47) as a white solid (11.2 g, 96%). R_{f}=0.3 (petroleum ether/ethyl acetate=1:1). 1H NMR (400 MHz, DMSO-d₆) δ (ppm): 7.26 (d, J=8.6 Hz, 2H), 6.93 (d, J=8.6 Hz, 2H), 5.40 (d, J=7.8 Hz, 1H), 5.32 (d, J=3.4 Hz, 1H), 5.27 (dd, J=10.4, 3.4 Hz, 1H), 5.22-5.18 (m, 1H), 5.17-5.13 (m, 1H), 4.41 (dd, J=13.6, 6.2 Hz, 3H), 4.09 (dd, J=6.2, 3.2 Hz, 2H), 2.14 (s, 3H), 2.03 (s, 3H), 2.00 (s, 3H), 1.94 (s, 3H). ESI-LCMS: m/z=472.2 [M+NH₄]⁺. calculated for C₂₁NH₃₀O₁₁=472.2.

47 (1.5 g, 3.3 mmol) was dissolved in dichloromethane (20 mL) and added to 4-nitrophenyl chloroformate (1.66 g, 8.25 mmol) and pyridine (260.7 mg, 3.3 mmol). The mixture was stirred at room temperature for 16 h. The mixture was poured into water and extracted with dichloromethane (3×30 mL). The combined organic layers were washed with brine (40 mL), dried over magnesium sulfate and concentrated. The residue was purified by flash chromatography (silica gel 50% ethyl acetate in petroleum ether) to afford (2R,3S,4S,5R,6S)-2-(acetoxymethyl)-6-(4-((((4-nitrophenoxy)carbonyl)oxy)methyl)phenoxy)tetrahydro-2H-pyran-3,4,5-triyl triacetate (48) as a white solid (1.79 g, 88%). R_{f}=0.65 (petroleum ether/ethyl acetate=1:1). ¹H NMR (400 MHz, DMSO-d₆) δ (ppm): 8.35-8.28 (m, 2H), 7.60-7.54 (m, 2H), 7.45 (d, J=8.6 Hz, 2H), 7.03 (d, J=8.6 Hz, 2H), 5.50 (d, J=7.7 Hz, 1H), 5.37-5.33 (m, 1H), 5.32-5.18 (m, 4H), 4.44 (t, J=6.4 Hz, 1H), 4.10 (d, J=6.2 Hz, 2H), 2.15 (s, 3H), 2.04 (s, 3H), 2.01 (s, 3H), 1.95 (s, 3H). ESI-LCMS: m/z=637.0 [M+NH₄]⁺. calculated for C₂₈H₃₃N₂O₁₅=637.2.

To a solution of 48 (2.3 g, 3.71 mmol) in dichloromethane (30 mL) was added tert-butyl methyl(2-(methylamino)ethyl)carbamate (1.46 g, 7.78 mmol) and DMAP (47.5 mg, 0.39 mmol). The mixture was stirred at room temperature for 3 h. The mixture was diluted with water (50 mL) and extracted with dichloromethane (2×50 mL). The combined organic phases were washed with brine (30 mL) and dried over magnesium sulfate. The filtrate was concentrated and dried under vacuum. The residue was purified by flash chromatography (silica gel; 33% ethyl acetate in petroleum ether) to afford (2R,3S,4S,5R,6S)-2-(acetoxymethyl)-6-(4-(4,7,10,10-tetramethyl-3,8-dioxo-2,9-dioxa-4,7-diazaundecyl)phenoxy)tetrahydro-2H-pyran-3,4,5-triyl triacetate (49) as a white solid (2.34 g, 94%). R_{f}=0.3 (petroleum ether/ethyl acetate=1:1). ¹H NMR (400 MHz, DMSO-d₆) δ (ppm): 7.32 (d, J=8.3 Hz, 2H), 7.00-6.94 (m, 2H), 5.45 (d, J=7.8 Hz, 1H), 5.36-5.32 (m, 1H), 5.28 (dd, J=10.2, 3.6 Hz, 1H), 5.20 (dd, J=10.2, 7.8 Hz, 1H), 4.98 (s, 2H), 4.42 (s, 1H), 4.15-4.06 (m, 2H), 3.29 (s, 2H), 2.90-2.65 (m, 6H), 2.15 (s, 3H), 2.03 (s, 3H), 2.00 (s, 3H), 1.95 (s, 3H), 1.35 (s, 9H). ESI-LCMS: m/z=691.4 [M+Na]⁺. calculated for C₃₁H₄₄N₂O₁₄Na=691.3.

A solution of 49 (1.0 g, 1.5 mmol) in 4 N hydrochloric acid in 1,4-dioxane (5 mL, 20 mmol) and acetonitrile (5 mL) was stirred at 0° C for 1 h. The solvent was removed under vacuum to afford crude (2R,3S,4S,5R,6S)-2-(acetoxymethyl)-6-(4-(((methyl(2-(methylamino)ethyl)carbamoyl)oxy)methyl)phenoxy)tetrahydro-2H-pyran-3,4,5-triyl triacetate hydrochloride (50) as a white solid (900 mg, crude), which was used directly in the next step without further purification. To a solution of tert-butyl (S)-1-(chloromethyl)-5-hydroxy-1,2-dihydro-3H-benzo[e]indole-3-carboxylate (14) (200 mg, 0.599 mmol) and 4-nitrophenyl chloroformate (181 mg, 0.898 mmol) in dichloromethane (5 mL) was added DIPEA (232 mg, 313 μL, 1.80 mmol) and the mixture was stirred at room temperature for 1 h. The mixture was diluted with water (60 mL) and extracted with dichloromethane (3×50 mL). The combined organic layers were washed with brine, dried over sodium sulfate, filtered and concentrated to afford tert-butyl (S)-1-(chloromethyl)-5-(((4-nitrophenoxy)carbonyl)oxy)-1,2-dihydro-3H-benzo[e]indole-3-carboxylate (15), which was used without further purification. The residue (15) was dissolved into DMF (5 mL) then 50 (682 mg, 1.2 mmol) and DIPEA (233 mg, 1.8 mmol) were added. The mixture was stirred at room temperature for overnight, diluted with water (60 mL) and extracted with ethyl acetate (2×60 mL). The combined organic layers were washed with brine (50 mL), dried over sodium sulfate, filtered and concentrated. The residue was purified by flash chromatography (silica gel; 0-50% ethyl acetate in petroleum ether) to afford (2R,3S,4S,5R,6S)-2-(acetoxymethyl)-6-(4-((((2-(((((S)-3-(tert-butoxycarbonyl)-1-(chloromethyl)-2,3-dihydro-1H-benzo[e]indol-5-yl)oxy)carbonyl)(methyl)amino)ethyl)(methyl)carbamoyl)oxy)methyl)phenoxy)tetrahydro-2H-pyran-3,4,5-triyl triacetate (51) as a yellow oil (400 mg, 72%). ¹H NMR (400 MHz, CDCl₃) δ (ppm): 8.15-7.97 (m, 1H), 7.89-7.74 (m, 1H), 7.73-7.66 (m, 1H), 7.58-7.26 (m, 4H), 7.02-6.82 (m, 2H), 5.51-5.42 (m, 2H), 5.14-4.92 (m, 4H), 4.34-4.09 (m, 4H), 4.07-3.89 (m, 3H), 3.79-3.42 (m, 5H), 3.26 (s, 1H), 3.11 (d, J=21.0 Hz, 1H), 3.03-2.87 (m, 5H), 2.18 (s, 3H), 2.07-2.04 (m, 6H), 2.01 (s, 3H), 1.57 (s, 9H). ESI-LCMS: m/z=950.5 [M+Na]⁺. calculated for C₄₅H₅₄ClN₃O₁₆Na=950.3.

To a solution of 51 (400 mg, 0.430 mmol) in acetonitrile (5 mL) at 0° C was added 4 N hydrochloric acid in 1,4-dioxane (5 mL, 20 mol). The reaction mixture was stirred at 0° C for 1 h. The solvent was removed under vacuum to afford (2R,3S,4S,5R,6S)-2-(acetoxymethyl)-6-(4-((((2-(((((S)-1-(chloromethyl)-2,3-dihydro-1H-benzo[e]indol-5-yl)oxy)carbonyl)(methyl)amino)ethyl)(methyl)carbamoyl)oxy)methyl)phenoxy)tetrahydro-2H-pyran-3,4,5-triyl triacetate (52) (930 mg, crude) as a brown oil, which was used directly in the next step. To a solution of 52 (360 mg, 0.434 mmol) in DMF (10 mL) was added glutaric anhydride (248 mg, 2.17 mmol), DMAP (80 mg, 0.651 mmol), and DIPEA (280 mg, 377 μL, 2.17 mmol). The reaction mixture was stirred at room temperature overnight. The mixture was diluted with water (100 mL) and extracted with ethyl acetate (3×50 mL). The combined organic layers were washed with brine (40 mL), dried over sodium sulfate, filtered and concentrated. The residue was purified by flash chromatography (silica gel; 0-95% methanol in dichloromethane) to afford 5-((S)-1-(chloromethyl)-5-((methyl(2-(methyl(((4-(((2S,3R,4S,5S,6R)-3,4,5-triacetoxy-6-(acetoxymethyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoic acid (53) as a yellow oil (200 mg, 49%). ¹H NMR (400 MHz, DMSO-d₆) δ (ppm): 8.20 (s, 1H), 7.95 (d, J=8.4 Hz, 1H), 7.89-7.71 (m, 1H), 7.65-7.23 (m, 4H), 6.88-6.89 (m, 2H), 5.37 (d, J=37.0 Hz, 2H), 5.29-5.15 (m, 2H), 5.04 (t, J=17.4 Hz, 2H), 4.43-4.17 (m, 4H), 4.11-3.86 (m, 4H), 3.78-3.47 (m, 6H), 2.91 (dd, J=32.4, 12.5 Hz, 4H), 2.34 (t, J=8.1 Hz, 2H), 2.23 (t, J=7.3 Hz, 2H), 2.14 (d, J=2.5 Hz, 3H), 2.06-1.91 (m, 9H), 1.83 (s, 2H). ESI-LCMS: m/z=964.5 [M+Na]⁺. calculated for C₄₅H₅₂ClN₃O₁₇Na=964.3.

A solution of 4-hydroxy-3-nitrobenzaldehyde (54) (5.0 g, 29.94 mmol) and (2R,3S,4S,5R,6R)-2-(acetoxymethyl)-6-bromotetrahydro-2H-pyran-3,4,5-triyl triacetate (45) (14.73 g, 35.93 mmol) in acetonitrile (50 mL) was stirred at room temperature overnight. The mixture was diluted with ethyl acetate (50 mL), washed with water (20 mL) and brine (20 mL), dried over sodium sulfate, filtered and concentrated. The residue was purified by trituration (petroleum ether/ethyl acetate=10:1) to afford (2R,3S,4S,5R,6S)-2-(acetoxymethyl)-6-(4-formyl-2-nitrophenoxy)tetrahydro-2H-pyran-3,4,5-triyl triacetate (55) as a yellow solid (15 g, 100%). ¹H NMR (400 MHz, DMSO-d₆) δ (ppm): 9.98 (s, 1H), 8.44 (d, J=2.0 Hz, 1H), 8.25 (dd, J=8.6, 2.2 Hz, 1H), 7.60 (d, J=8.8 Hz, 1H), 5.79 (d, J=6.4, 1.3 Hz, 1H), 5.44-5.36 (m, 1H), 5.32-5.24 (m, 2H), 4.55 (t, 1H), 4.19-4.09 (m, 2H), 2.15 (s, 3H), 2.04 (d, J=2.2 Hz, 6H), 1.95 (s, 3H). ESI-LCMS: m/z=515.1 [M+NH₄]⁺; calculated for C₂₁H₂₇N₂O₁₃=515.2.

To a solution of 55 (15 g, 30.1 mmol) in dichloromethane/isopropanol (4:1, 220 mL) was added sodium borohydride (1.26 g, 33.15 mmol) and silica gel (6.6 g). After stirring at room temperature overnight, the mixture was filtered. The filtrate washed with water (100 mL) and extracted with dichloromethane (3×40 mL). The combined organic phases were washed with water (40 mL) and brine (40 mL), dried over sodium sulfate and concentrated. The residue was purified by flash chromatography (silica gel; petroleum ether/ethyl acetate/dichloromethane=50:1:1 to 1:1:2) to afford (2R,3S,4S,5R,6S)-2-(acetoxymethyl)-6-(4-(hydroxymethyl)-2-nitrophenoxy)tetrahydro-2H-pyran-3,4,5-triyl triacetate (56) as a yellow solid (7 g, 46%). ¹H NMR (400 MHz, CDCl₃) δ (ppm): 7.79 (d, J=2.2 Hz, 1H), 7.50 (dd, J=8.6 Hz, 2.2 Hz, 1H), 7.34 (d, J=8.6 Hz, 1H), 5.55-5.48 (m, 1H), 5.48-5.43 (m, 1H), 5.09 (dd, J=10.6, 3.4 Hz, 1H), 5.04 (d, J=7.8 Hz, 1H), 4.71 (d, J=5.6 Hz, 2H), 4.29-4.11 (m, 2H), 4.08-4.04 (m, 1H), 2.18 (s, 3H), 2.15 (t, J=5.6 Hz, 1H), 2.12 (s, 3H), 2.06 (s, 3H), 2.00 (s, 3H). ESI-LCMS: m/z=522.3 [M+Na]⁺. calculated for C₂₁H₂₅NO₁₃Na=522.1.

To a solution of 56 (3 g, 6 mmol) dichloromethane (96 mL) was added 4-nitrophenyl chloroformate (3.03 g, 15.02 mmol) and pyridine (713 mg, 729 μL, 9.02 mmol). After stirring at room temperature overnight, the mixture was poured into water and extracted with dichloromethane (3×10 mL). The combined organic layers were washed with water (15 mL) and brine (15 mL), dried over sodium sulfate and concentrated in vacuo. The residue was purified by flash chromatography (silica gel; petroleum ether/ethyl acetate/dichloromethane=50:1:1 to 2:1:1) to afford (2R,3S,4S,5R,6S)-2-(acetoxymethyl)-6-(2-nitro-4-((((4-nitrophenoxy)carbonyl)oxy)methyl)phenoxy)tetrahydro-2H-pyran-3,4,5-triyl triacetate (57) as a white solid (2.67 g, 67%). ¹H NMR (400 MHz, DMSO-d₆) δ=8.36-8.29 (m, 2H), 8.04 (d, J=2.2 Hz, 1H), 7.83 (dd, J=8.8, 2.2 Hz, 1H), 7.62-7.55 (m, 2H), 7.46 (d, J=8.8 Hz, 1H), 5.63 (d, J=7.4 Hz, 1H), 5.42-5.21 (m, 5H), 4.50 (t, J=6.6 Hz, 1H), 4.21-4.08 (m, 2H), 2.15 (s, 3H), 2.04 (s, 4H), 1.98 (s, 2H), 1.95 (s, 3H). ESI-LCMS: m/z=687.3 [M+Na]⁺. calculated for C₂₈H₂₈N₂O₁₇Na=687.1.

To a solution of (2R,3S,4S,5R,6S)-2-(acetoxymethyl)-6-(2-nitro-4-((((4-nitrophenoxy)carbonyl)oxy)methyl)phenoxy)tetrahydro-2H-pyran-3,4,5-triyl triacetate (57) (4 g, 6.02 mmol) in DMF (80.0 mL) was added tert-butyl methyl(2-(methylamino)ethyl)carbamate (11) (1.70 g, 9.03 mmol) and DIPEA (1.56 g, 2.10 mL, 12.07 mmol). After stirring at room temperature for 2 h, the mixture was poured into water and extracted with ethyl acetate (3×10 mL). The combined organic layers were washed with water and brine, dried over sodium sulfate and concentrated in vacuo. The residue was purified by reverse-phase flash chromatography (60% acetonitrile in water) to afford (2R,3S,4S,5R,6S)-2-(acetoxymethyl)-6-(2-nitro-4-(4,7,10,10-tetramethyl-3,8-dioxo-2,9-dioxa-4,7-diazaundecyl)phenoxy)tetrahydro-2H-pyran-3,4,5-triyl triacetate (58) as a yellow oil (3 g, 70%). ¹H NMR (400 MHz, DMSO-d₆) δ (ppm): 7.88 (s, 1H), 7.69 (d, 1H), 7.40 (d, J=8.8 Hz, 1H), 5.58 (d, J=7.6 Hz, 1H), 5.40-5.35 (m, 1H), 5.30-5.21 (m, 2H), 5.10-5.02 (m, 2H), 4.48 (t, J=6.4 Hz, 1H), 4.19-4.08 (m, 2H), 3.34 (s, 4H), 2.79 (dd, J=49.6, 16.8 Hz, 6H), 2.15 (s, 3H), 2.05-1.97 (m, 9H), 1.38-1.27 (m, 9H). ESI-LCMS: m/z=614.2 [(M-Boc)+H]⁺. calculated for C₂₆H₃₆N₃O₁₄=614.2.

To a solution of 58 (100 mg, 140.25 μmol) in ethyl acetate (5.0 mL) was added Pd/C (10 wt. % loading, 5 mg, 5%). After stirring at room temperature for 1.5 h under H₂ atmosphere, the mixture was filtered and concentrated in vacuo. The residue was purified by preparative TLC (petroleum ether/ethyl acetate=1:1) to afford (2R,3S,4S,5R,6S)-2-(acetoxymethyl)-6-(2-amino-4-(4,7,10,10-tetramethyl-3,8-dioxo-2,9-dioxa-4,7-diazaundecyl)phenoxy)tetrahydro-2H-pyran-3,4,5-triyl triacetate (59) as a yellow oil (50 mg, 52%). ¹H NMR (400 MHz, CDCl₃) δ (ppm): 7.22-6.74 (m, 2H), 5.61-5.40 (m, 2H), 5.19-4.93 (m, 3H), 4.36-3.97 (m, 4H), 3.52-3.17 (m, 4H), 2.98-2.89 (m, 3H), 2.89-2.75 (m, 3H), 2.20-2.01 (m, 13H), 1.52-1.36 (m, 9H). ESI-LCMS: m/z=684.5 [M+H]⁺. calculated for C₃₁H₄₆N₃O₁₄=684.2.

To a solution of 59 (800 mg, 1.17 mmol) and 1-(6-methyl-4-vinylpyridin-2-yl)-3-oxo-7,10,13,16,19,22,25,28-octaoxa-4-azahentriacontan-31-oic acid (24) (838 mg, 1.29 mmol) in DMF (20.0 mL) were added DIPEA (302 mg, 407 μL, 2.34 mmol) and HATU (667 mg, 1.76 mmol). After stirring at room temperature overnight, the mixture was poured into water (160 mL) and extracted with ethyl acetate (20 mL×4). The combined organic layers were washed with water (30 mL) and brine (30 mL), dried over sodium sulfate and concentrated in vacuo. The residue was purified by reverse-phase flash chromatography (65% acetonitrile in water) to afford 60 as a yellow oil (980 mg, 65%). ¹H NMR (400 MHz, CDCl₃) δ (ppm): 8.45 (s, 1H), 8.09-8.02 (m, 1H), 7.63 (s, 1H), 7.42 (s, 1H), 7.02 (d, J=8.2 Hz, 1H), 6.94 (d, J=8.2 Hz, 1H), 6.82-6.70 (m, 1H), 6.31 (d, J=17.6 Hz, 1H), 5.88 (d, J=10.8 Hz, 1H), 5.53-5.41 (m, 2H), 5.17 (dd, 1H), 5.08-4.99 (m, 3H), 4.26-4.14 (m, 2H), 3.89-3.78 (m, 2H), 3.68-3.58 (m, 29H), 3.51-3.45 (m, 2H), 3.42-3.32 (m, 6H), 2.94 (s, 5H), 2.87 (s, 1H), 2.84-2.78 (m, 4H), 2.71 (t, 2H), 2.41 (s, 4H), 2.20 (s, 3H), 2.08 (d, J=9.2 Hz, 6H), 2.05-2.04 (m, 3H), 1.44 (s, 9H). ESI-LCMS: m/z=1280.6 [M+H]⁺. calculated for C₆₁H₉₄N₅O₂₄=1280.6.

A solution of 60 (100 mg, 78.15 μmol) in 4 N hydrochloric acid in 1,4-dioxane (2 mL) and acetonitrile (2 mL) was stirred at 0° C for 1 h. The mixture was concentrated in vacuo to afford crude 61 as a yellow oil (100 mg, crude), which was used to next step directly. ESI-LCMS: m/z=1180.8 [M+H]⁺. calculated for C₅₆H₈₆N₅O₂₂=1180.6.

To a solution of tert-butyl (S)-1-(chloromethyl)-5-(((4-nitrophenoxy)carbonyl)oxy)-1,2-dihydro-3H-benzo[e]indole-3-carboxylate (15) (269 mg, 0.539 mmol) in DMF (10 mL) was added 61 (922 mg, 0.781 mmol) and DIPEA (202 mg, 272 μL, 1.56 mmol). The mixture was stirred at room temperature overnight. The mixture was diluted with water (60 mL) and extracted with ethyl acetate (2×60 mL). The combined organic layers were washed with brine, dried over sodium sulfate, filtered and concentrated. The residue obtained was purified by flash chromatography (silica gel; 0-6% methanol in dichloromethane) to afford 62 as a yellow oil (650 mg, 78%). ESI-LCMS: m/z=770.9 [M+2H]²⁺. calculated for C₇₅H₁₀₅ClN₆O₂₆=1540.7.

To a solution of 62 (550 mg, 0.357 mmol) in acetonitrile (10 mL) at 0° C was added a solution of 4 N hydrochloric acid in 1,4-dioxane (10 mL). The reaction mixture was stirred at 0° C for 1 h. The solvent was removed under vacuum to afford crude (2R,3S,4S,5R,6S)-2-(acetoxymethyl)-6-(4-((((2-(((((S)-1-(chloromethyl)-2,3-dihydro-1H-benzo[e]indol-5-yl)oxy)carbonyl)(methyl)amino)ethyl)(methyl)carbamoyl)oxy)methyl)-2-(1-(6-methyl-4-vinylpyridin-2-yl)-3-oxo-7,10,13,16,19,22,25,28-octaoxa-4-azahentriacontan-31-amido)phenoxy)tetrahydro-2H-pyran-3,4,5-triyl triacetate (63) as a brown oil (560 mg, crude), which was used directly in next step without further purification.

To 5-((S)-1-(chloromethyl)-5-((methyl(2-(methyl(((4-(((2S,3R,4S,5S,6R)-3,4,5-triacetoxy-6-(acetoxymethyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoic acid (53) (75 mg, 0.079 mmol) and 63 (229 mg, 0.159 mmol) in anhydrous DMF (5 mL) was added EDCl (76 mg, 0.398 mmol). The reaction mixture stirred at room temperature overnight. The mixture was diluted with water (60 mL) and extracted with ethyl acetate (3×50 mL). The combined organic layers were washed with brine (30 mL), dried over sodium sulfate, filtered and concentrated. The residue obtained was purified by preparative TLC (dichloromethane:methanol=15:1) to afford (2R,3S,4S,5R,6S)-2-(acetoxymethyl)-6-(4-((((2-(((((S)-1-(chloromethyl)-3-(5-((S)-1-(chloromethyl)-5-((methyl(2-(methyl(((3-(1-(6-methyl-4-vinylpyridin-2-yl)-3-oxo-7,10,13,16,19,22,25,28-octaoxa-4-azahentriacontan-31-amido)-4-(((2S,3R,4S,5S,6R)-3,4,5-triacetoxy-6-(acetoxymethyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)phenoxy)tetrahydro-2H-pyran-3,4,5-triyl triacetate (64) as a yellow oil (175 mg, 47%). ESI-LCMS: m/z=1183 [M+2H]²⁺. calculated for C₁₁₅H₁₄₇Cl₂N₉O₄₀=2363.9.

To a solution of 64 (100 mg, 0.042 mmol) in acetonitrile (1.5 mL) and water (1.5 mL) at 0° C was added lithium hydroxide hydrate (36 mg, 0.846 mmol). The mixture was stirred at 0° C for 2 h, acidified with 2 N hydrochloric acid to pH~3, purified by preparative HPLC to afford (S)-1-(chloromethyl)-3-(5-((S)-1-(chloromethyl)-5-((methyl(2-(methyl(((3-(1-(6-methyl-4-vinylpyridin-2-yl)-3-oxo-7,10,13,16,19,22,25,28-octaoxa-4-azahentriacontan-31-amido)-4-(((2S,3R,4S,5R,6R)-3,4,5-trihydroxy-6-(hydroxymethyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoyl)-2,3-dihydro-1H-benzo[e]indol-5-yl (4-(((2S,3R,4S,5R,6R)-3,4,5-trihydroxy-6-(hydroxymethyl)tetrahydro-2H-pyran-2-yl)oxy)benzyl) ethane-1,2-diylbis(methylcarbamate) (3) as a white solid (16.1 mg, 19%). ¹H NMR (400 MHz, DMSO-d₆) δ (ppm): 9.17-9.09 (m, 1H), 8.32-8.16 (m, 3H), 7.99-7.71 (m, 5H), 7.63-7.53 (m, 2H), 7.49-7.19 (m, 4H), 7.17-6.86 (m, 5H), 6.65 (dd, J=17.8, 10.8 Hz, 1H), 6.05 (d, J=17.6 Hz, 1H), 5.68 (s, 1H), 5.46 (d, J=10.8 Hz, 1H), 5.17-4.73 (m, 8H), 4.70-4.16 (m, 11H), 4.09-3.83 (m, 4H), 3.79-3.62 (m, 8H), 3.61-3.42 (m, 42H), 3.41-3.36 (m, 7H), 3.21-3.08 (m, 5H), 3.00-2.83 (m, 10H), 2.76-2.58 (m, 5H), 2.48-2.45 (m, 1H), 2.42-2.40 (m, 2H), 2.04-1.88 (m, 2H). ESI-LCMS: m/z=1014.7 [M+2H]²⁺. calculated for C₉₉H₁₃₁Cl₂N₉O₃₂=2027.8.

### Preparation of Compound 4

N—((S)-1-(((S)-1-((4-((((S)-1-(chloromethyl)-3-(5-((S)-1-(chloromethyl)-5-(((2S,3R,4S,5R,6R)-3,4,5-trihydroxy-6-(hydroxymethyl)tetrahydro-2H-pyran-2-yl)oxy)-1,2-dihydro-3H-benzo[e]indol-3-yl)-5-oxopentanoyl)-2,3-dihydro-1H-benzo[e]indol-5-yl)oxy)methyl)phenyl)amino)-1-oxo-5-ureidopentan-2-yl)amino)-3-methyl-1-oxobutan-2-yl)-1-(3-(6-methyl-4-vinylpyridin-2-yl)propanamido)-3,6,9,12,15,18,21,24-octaoxaheptacosan-27-amide (4), shown above, may be synthesised according to methods known in the art.

### Preparation of Compound 5

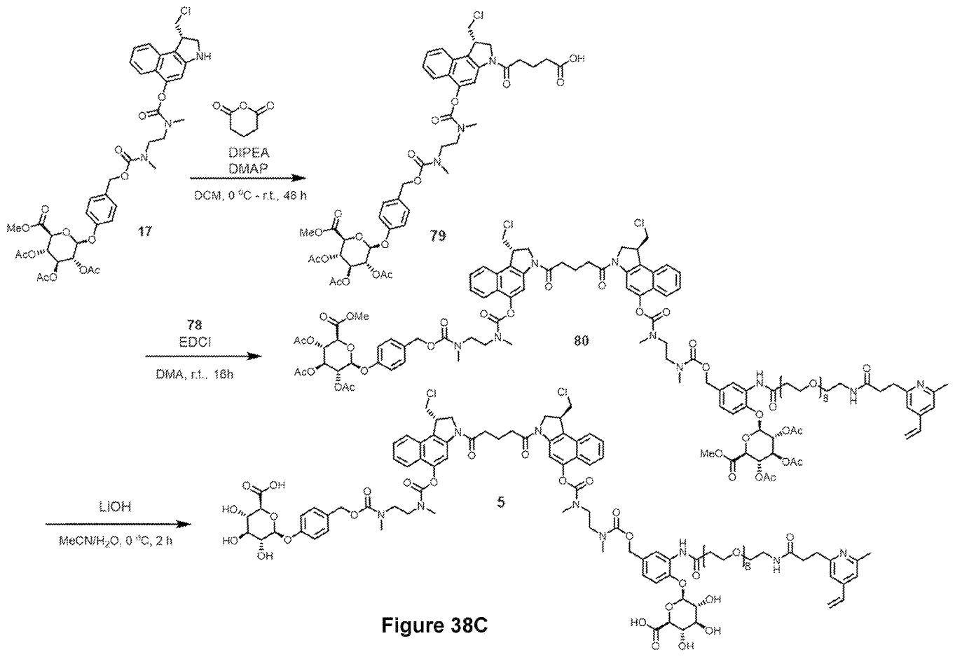

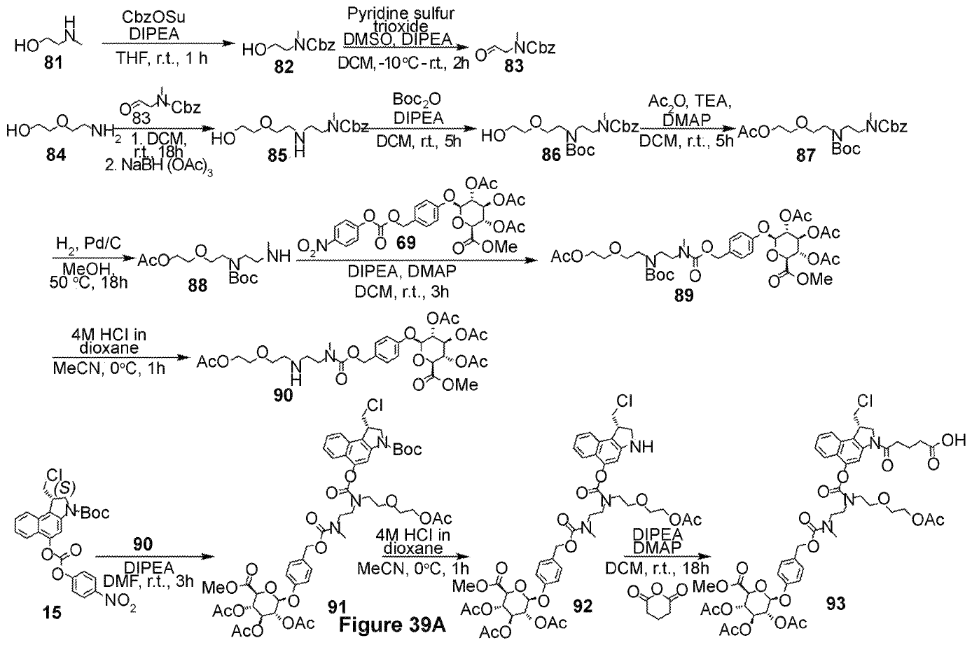

(2S,3S,4S,5R,6S)-6-(4-((((2-(((((S)-3-(5-((S)-5-(((2-((((4-(((2S,3R,4S,5S,6S)-6-carboxy-3,4,5-trihydroxytetrahydro-2H-pyran-2-yl)oxy)-3-(1-(6-methyl-4-vinylpyridin-2-yl)-3-oxo-7,10,13,16,19,22,25,28-octaoxa-4-azahentriacontan-31-amido)benzyl)oxy)carbonyl)(methyl)amino)ethyl)(methyl)carbamoyl)oxy)-1-(chloromethyl)-1,2-dihydro-3H-benzo[e]indol-3-yl)-5-oxopentanoyl)-1-(chloromethyl)-2,3-dihydro-1H-benzo[e]indol-5-yl)oxy)carbonyl)(methyl)amino)ethyl)(methyl)carbamoyl)oxy)methyl)phenoxy)-3,4,5-trihydroxytetrahydro-2H-pyran-2-carboxylic acid (5) was prepared according to the schemes shown in FIGS. 38A to 38C.

To a solution of 4-hydroxybenzaldehyde (44) (10.00 g, 81.89 mmol) in acetonitrile (300 mL) was added (2R,3R,4S,5S,6S)-2-bromo-6-(methoxycarbonyl)tetrahydro-2H-pyran-3,4,5-triyl triacetate (66) (35.78 g, 90.08 mmol) and silver (1) oxide (37.95 g, 163.78 mmol). The mixture was stirred at room temperature for 2 h. The mixture was filtrated and washed with dichloromethane (200 mL). The filtrate was concentrated. The residue was triturated with petroleum ether/ethyl acetate=(100 mL, 1:1) and the solid was collected by filtration and dried under reduced pressure to afford (2S,3R,4S,5S,6S)-2-(4-formylphenoxy)-6-(methoxycarbonyl)tetrahydro-2H-pyran-3,4,5-triyl triacetate (67) as a light-yellow solid (26.0 g, 72%). TLC: R_{f}=0.3 (petroleum ether/ethyl acetate=2:1). LCMS: Theoretical: C₂₀H₂₂O₁₁ 438.12, Observed: m/z 456.10 [M+NH₄]⁺. ¹H NMR (400 MHz, DMSO-d₆) δ (ppm): 9.92 (s, 1H), 7.95-7.90 (m, 2H), 7.24-7.17 (m, 2H), 5.86 (d, J=7.8 Hz, 1H), 5.49 (t, J=9.6 Hz, 1H), 5.21-5.07 (m, 2H), 4.77 (d, J=9.8 Hz, 1H), 3.64 (s, 3H), 2.05-1.99 (m, 9H).

To a solution of 67 (20.00 g, 45.62 mmol) and silica-gel (10 g) in dichloromethane/isopropanol (200 mL/100 mL) at −10° C was added NaBH₄ (1.90 g, 50.18 mmol) slowly. The mixture was stirred at 0° C for 30 min. The mixture was diluted with ethyl acetate and filtrated. The filtrate was poured into water (400 mL) and extracted with ethyl acetate (200 mL×3). The combined organic phases were washed with brine (1 L), dried over Na₂SO₄ and concentrated. The residue was purified by column chromatography on silica gel (eluent: petroleum ether/ethyl acetate=2:1) to afford (2S,3R,4S,5S,6S)-2-(4-(hydroxymethyl)phenoxy)-6-(methoxycarbonyl)tetrahydro-2H-pyran-3,4,5-triyl triacetate (9) as a white solid (18.8 g, 52%). TLC: R_{f}=0.3 (petroleum ether/ethyl acetate=1:1). LCMS: Theoretical: C₂₀H₂₄O₁₁ 440.13, Observed: m/z 458.10 [M+NH₄]⁺. ¹H NMR (400 MHz, DMSO-d₆) δ (ppm): 7.20-7.15 (d, 2H, J=8.8 Hz), 6.89-6.83 (d, 2H, J=8.8 Hz), 5.53 (d, J=7.8 Hz, 1H), 5.37 (t, J=9.6 Hz, 1H), 5.06-4.93 (m, 3H), 4.60 (d, J=9.8 Hz, 1H), 4.34 (d, J=5.6 Hz, 2H), 3.55 (s, 3H), 1.94-1.90 (m, 9H).

To solution of 9 (18.80 g, 42.69 mmol) in THF (300 mL) was added bis(4-nitrophenyl) carbonate (26.00 g, 85.36 mmol) and DIPEA (16.60 g, 22.4 mL, 128.07 mmol). The mixture was stirred at room temperature for 16 h. The mixture was diluted with ethyl acetate (1.5 L) and aq. NaHCO₃ (10%, 1.5 L). The ethyl acetate layer was washed with brine (1 L) and dried over MgSO₄. The filtrate was concentrated and dried under vacuum. The obtained residue was purified by column chromatography on silica gel (eluent: petroleum ether/ethyl acetate=5:1 to dichloromethane/petroleum ether=2:1) to afford (2S,3S,4S,5R,6S)-2-(Methoxycarbonyl)-6-(4-((((4-nitrophenoxy)carbonyl)oxy)methyl)phenoxy)tetrahydro-2H-pyran-3,4,5-triyl triacetate (69) as a white solid (22.2 g, 86%). TLC: R_{f}=0.5 (petroleum ether/ethyl acetate=1:1). LCMS: Theoretical: C₂₇H₂₇NO₁₅ 605.14, Observed: m/z 623.05 [M+NH₄]⁺. ¹H NMR (400 MHz, DMSO-d₆) δ (ppm): 8.33-8.29 (d, 2H, J=8.4 Hz), 7.60-7.53 (d, 2H, J=8.4 Hz), 7.48-7.42 (d, 2H, J=6.8 Hz), 7.07-7.02 (d, 2H, J=6.8 Hz), 5.70 (d, J=7.8 Hz, 1H), 5.47 (t, J=9.6 Hz, 1H), 5.25 (s, 2H), 5.13-5.04 (m, 2H), 4.72 (d, J=9.8 Hz, 1H), 3.64 (s, 3H), 2.02-1.99 (m, 9H).

To a solution of 69 (22.20 g, 36.66 mmol) in dichloromethane (300 mL) was added tert-butyl methyl(2-(methylamino)ethyl)carbamate (11) (13.80 g, 73.32 mmol) and DMAP (448 mg, 3.67 mmol). The mixture was stirred at room temperature for 2 h. The mixture was diluted with water (1.5 L) and extracted with ethyl acetate (1.5 L). The ethyl acetate layer was washed with brine (1 L) and dried over MgSO₄. The filtrate was concentrated and dried under vacuum. The obtained residue was purified by column chromatography on silica gel (eluent petroleum ether/ethyl acetate=10:1 to dichloromethane/methanol=50:1) to afford (2S,3S,4S,5R,6S)-2-(methoxycarbonyl)-6-(4-(4,7,10,10-tetramethyl-3,8-dioxo-2,9-dioxa-4,7-diazaundecyl)phenoxy)tetrahydro-2H-pyran-3,4,5-triyl triacetate (12) as a light-yellow solid (13.50 g, 56%). TLC: R, =0.3 (petroleum ether/ethyl acetate=1:1). LCMS: Theoretical: C₃₀H₄₂N₂O₁₄654.26, Observed: m/z 672.20 [M+NH₄]. ¹H NMR (400 MHz, Chloroform-d) δ (ppm): 7.29 (d, J=8.6 Hz, 2H), 6.99-6.94 (m, 2H), 5.36-5.30 (m, 2H), 5.26 (td, J=7.0, 2.8 Hz, 1H), 5.13 (d, J=7.2 Hz, 1H), 5.05 (s, 2H), 4.20-4.13 (m, 1H), 3.72 (s, 3H), 3.37-3.30 (m, 4H), 2.93 (d, J=6.7 Hz, 3H), 2.86-2.78 (m, 3H), 2.06-2.02 (m, 9H), 1.43 (s, 9H).

A solution of 12 (2.0 g, 3.05 mmol) in acetonitrile (10 mL) at 0° C was added 4 N hydrochloric acid/1,4-dioxane (10.0 mL). The mixture was stirred at 0° C for 1 h. The mixture was concentrated under vacuum and the residue was triturated with diethyl ether to afford (2S,3S,4S,5R,6S)-2-(methoxycarbonyl)-6-(4-(((methyl(2-(methylamino)ethyl)carbamoyl)oxy)methyl)phenoxy)tetrahydro-2H-pyran-3,4,5-triyl triacetate (13) as a white solid (1.8 g, crude). LCMS: Theoretical: C₂₅H₃₄N₂O₁₂ 554.21, Observed: m/z 555.35 [M+H]⁺.

To a solution of tert-butyl(S)-5-(benzyloxy)-1-(chloromethyl)-1,2-dihydro-3H-benzo[e]indole-3-carboxylate (31) (500 mg, 1.18 mmol) in dichloromethane (8.0 mL) and methanol (8.0 mL) was added 10% Pd/C (150 mg). After stirring at room temperature for 20 h under H₂ atmosphere, the mixture was filtered and the filtrate was concentrated under vacuum. The crude product was purified by column chromatography on silica gel (eluent: petroleum ether/ethyl acetate=100:1 to 30:1) to obtain tert-butyl (S)-1-(chloromethyl)-5-hydroxy-1,2-dihydro-3H-benzo[e]indole-3-carboxylate (14) as a white solid (330 mg, 84%). LCMS: Theoretical: C₁₈H₂₀ClNO₃ 333.11, Observed: m/z 334.25 [M+H]⁺. ¹H NMR (400 MHz, DMSO-d₆) δ (ppm): 10.34 (s, 1H), 8.06 (d, J=8.2 Hz, 1H), 7.83-7.52 (m, 2H), 7.47 (t, J=8.0 Hz, 1H), 7.28 (m, 1H), 4.18-3.91 (m, 4H), 3.79-3.70 (m, 1H), 1.54 (s, 9H).

To a solution of 14 (200 mg, 599 μmol) and DIPEA (231 mg, 331 μL, 1.79 mmol) in dichloromethane (5.0 mL) was added 4-nitrophenyl chloroformate (181 mg, 898 μmol). The mixture was stirred at room temperature for 1 h. The mixture was poured into water (20 mL) and extracted with dichloromethane (20 mL×3). The combined organic layers were dried over Na₂SO₄ and concentrated under vacuum to afford tert-butyl (S)-1-(chloromethyl)-5-(((4-nitrophenoxy)carbonyl)oxy)-1,2-dihydro-3H-benzo[e]indole-3-carboxylate (15) as a light-yellow oil (298 mg, crude), which was used to next step directly.

The crude residue of 15 (298 mg, 0.599 mmol) was dissolved in DMF (5 mL), then 13 (727 mg, 1.3 mmol) and DIPEA (233 mg, 314 μL, 1.8 mmol) were added. The mixture was stirred at room temperature for overnight, diluted with water (200 mL) and extracted with ethyl acetate (60 mL×3). The combined organic layers were washed with brine (40 mL), dried over Na₂SO₄, filtered and concentrated. The residue obtained was purified by prep-TLC (dichloromethane/methanol=15:1) to afford (2S,3R,4S,5S,6S)-2-(4-((((2-(((((S)-3-(tert-butoxycarbonyl)-1-(chloromethyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (16) as a white solid (450 mg, 82%). LCMS: Theoretical: C₄₄H₅₂ClN₃O₁₆ 913.30, Observed: m/z 931.6 [M+NH₄]. ¹H NMR (400 MHz, DMSO-d₆) δ (ppm): 8.13-7.17 (m, 7H), 7.04-6.77 (m, 2H), 5.66-5.52 (m, 1H), 5.50-5.40 (m, 1H), 5.13-4.94 (m, 4H), 4.73-4.62 (m, 1H), 4.28-4.14 (m, 2H), 4.12-3.99 (m, 2H), 3.95-3.84 (m, 1H), 3.79-3.68 (m, 1H), 3.63 (s, 3H), 3.59-3.40 (m, 3H), 3.20-3.10 (m, 1H), 3.00-2.82 (m, 5H), 2.02-1.97 (m, 10H), 1.55-1.48 (m, 9H).

To a solution of 16 (450 mg, 0.49 mmol) in acetonitrile (5 mL) at 0° C was added 4 N hydrochloric acid/1,4-dioxane (5 mL). The mixture was stirred at 0° C for 1 h. The mixture was concentrated under vacuum and the residue was triturated with diethyl ether to afford (2S,3R,4S,5S,6S)-2-(4-((((2-(((((S)-1-(chloromethyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (17) as a brown solid (400 mg, crude). LCMS: Theoretical: C₃₉H₄₄ClN₃O₁₄ 813.25, Observed: m/z 814.45 [M+H]⁺.

To a solution of 4-hydroxy-3-nitrobenzaldehyde (54) (10.0 g, 59.8 mmol) in acetonitrile (100 mL) was added 66 (28.5 g, 71.8 mmol) and silver (1) oxide (27.7 g, 119.7 mmol). The mixture was stirred at room temperature overnight. The reaction mixture was filtered and concentrated. The residue obtained was triturated with petroleum ether to afford (2S,3R,4S,5S,6S)-2-(4-formyl-2-nitrophenoxy)-6-(methoxycarbonyl)tetrahydro-2H-pyran-3,4,5-triyl triacetate (70) as a white solid (24 g, 83%). TLC: R, =0.4 (petroleum ether/ethyl acetate=1:1). LCMS: Theoretical: C₂₀H₂₁NO₁₃ 483.10, Observed m/z 501.20 [M+NH₄]⁺. ¹H NMR (400 MHz, DMSO-d₆) δ (ppm): 9.98 (s, 1H), 8.44 (d, J=2.0 Hz, 1H), 8.22 (dd, J=8.6 Hz, 1H), 7.64 (d, J=8.6 Hz, 1H), 5.93 (d, J=7.6 Hz, 1H), 5.47 (t, J=9.4 Hz, 1H), 5.23-5.09 (m, 2H), 4.80 (d, J=9.6 Hz, 1H), 3.63 (s, 3H), 2.07-1.96 (m, 9H).

To solution of 70 (28 g, 57.9 mmol) in dichloromethane (55 mL) was added silica gel (14 g) and NaBH₄ (2.4 g, 63.7 mmol). The reaction mixture was stirred at 0° C for 30 min. The mixture was quenched with water (400 mL) and extracted with dichloromethane (100 mL×3). The combined organic layers were dried over Na₂SO₄, filtered and concentrated. The residue obtained was purified by column chromatography on silica gel (eluent petroleum ether/ethyl acetate=15:1 to 1:1) to afford (2S,3R,4S,5S,6S)-2-(4-(hydroxymethyl)-2-nitrophenoxy)-6-(methoxycarbonyl)tetrahydro-2H-pyran-3,4,5-triyl triacetate (71) as a white solid (23 g, 82%). TLC: R_{f}=0.3 (petroleum ether/ethyl acetate=1:2). LCMS: Theoretical: C₂₀H₂₃NO₁₃ 485.12, Observed: m/z 503.30 [M+NH₄]. ¹H NMR (400 MHz, DMSO-d₆) δ (ppm): 7.80 (d, J=2.0 Hz, 1H), 7.62 (dd, J=8.6, 2.1 Hz, 1H), 7.38 (d, J=8.6 Hz, 1H), 5.71 (d, J=7.8 Hz, 1H), 5.52-5.37 (m, 2H), 5.17-5.06 (m, 2H), 4.73 (d, J=9.8 Hz, 1H), 4.51 (d, J=5.6 Hz, 2H), 3.65 (s, 3H), 2.00 (d, J=8.4 Hz, 9H).

To a solution of 71 (23 g, 47.4 mmol) in THF (100 mL) was added bis(4-nitrophenyl) carbonate (28.8 g, 94.8 mmol), DIPEA (12.2 g, 127.2 mL, 94.8 mmol) and the mixture stirred at room temperature overnight under N₂ atmosphere. The mixture was diluted with ethyl acetate (300 mL) and washed with 10% NaHSO₃ (100 mL×3) and brine (100 mL), dried over Na₂SO₄, filtered and concentrated. The residue obtained was purified by column chromatography on silica gel (eluent: petroleum ether/ethyl acetate/dichloromethane=10:1:1 to 1:1:1) to afford (2S,3S,4S,5R,6S)-2-(methoxycarbonyl)-6-(2-nitro-4-((((4-nitrophenoxy)carbonyl)oxy)methyl)phenoxy)tetrahydro-2H-pyran-3,4,5-triyl triacetate (72) as a white solid (21 g, 68%). TLC: R_{f}=0.6 (petroleum ether/ethyl acetate=1:1). LCMS: Theoretical: C₂₇H₂₆N₂O₁₇ 650.12, m/z 668.30 [M+NH₄]⁺. ¹H NMR (400 MHz, DMSO-d₆) δ (ppm): 8.32 (dd, J=9.2, 2.8 Hz, 2H), 8.05 (s, 1H), 7.81 (dd, J=8.6, 2.4 Hz, 1H), 7.58 (dd, J=10.0, 3.2 Hz, 2H), 7.48 (d, J=8.4 Hz, 1H), 5.85-5.76 (m, 1H), 5.51-5.44 (m, 1H), 5.33 (s, 2H), 5.20-5.01 (m, 2H), 4.76 (dd, J=9.8, 2.4 Hz, 1H), 3.65 (s, 3H), 2.12-1.92 (m, 9H).

To a solution of 72 (5.0 g, 7.7 mmol) in dichloromethane (30 mL) was added tert-butyl methyl(2-(methylamino)ethyl)carbamate (11) (2.9 g, 15.4 mmol) and DMAP (94.0 mg, 0.77 mmol). The mixture was stirred at room temperature for 2 h. The reaction mixture was diluted with dichloromethane (200 mL), washed with 2 N hydrochloric acid (70 mL×2) and brine (50 mL). The organic phase was dried over Na₂SO₄, filtered and concentrated. The residue was purified by column chromatography on silica gel (eluent: petroleum ether/ethyl acetate=5:1 to 1:2) to afford (2S,3S,4S,5R,6S)-2-(methoxycarbonyl)-6-(2-nitro-4-(4,7,10,10-tetramethyl-3,8-dioxo-2,9-dioxa-4,7-diazaundecyl)phenoxy)tetrahydro-2H-pyran-3,4,5-triyl triacetate (73) as a yellowish solid (4.0 g, 74%). TLC: R_{f}=0.3 (petroleum ether/ethyl acetate=1:2). ¹H NMR (400 MHz, DMSO-d₆) δ (ppm): 7.95-7.85 (m, 1H), 7.74-7.64 (m, 1H), 7.47-7.37 (m, 1H), 5.74 (d, J=7.8 Hz, 1H), 5.46 (t, J=9.6 Hz, 1H), 5.16-5.05 (m, 3H), 4.74 (d, J=9.8 Hz, 1H), 3.64 (s, 3H), 3.38-3.34 (m, 2H), 3.31-3.25 (m, 2H), 2.85 (d, J=15.8 Hz, 3H), 2.79-2.68 (m, 3H), 2.03-2.00 (m, 6H), 2.00-1.99 (m, 2H), 1.38-1.29 (m, 9H).

To solution of 73 (4.0 g, 5.7 mmol) in ethyl acetate (30 mL) was added 10% Pd/C (800 mg). The mixture was stirred under a H₂ atmosphere for 24 h. The mixture was filtered and concentrated. The residue obtained was purified by column chromatography on silica gel (eluent petroleum ether/ethyl acetate=2:1 to 1:2) to afford (2S,3R,4S,5S,6S)-2-(2-amino-4-(4,7,10,10-tetramethyl-3,8-dioxo-2,9-dioxa-4,7-diazaundecyl)phenoxy)-6-(methoxycarbonyl)tetrahydro-2H-pyran-3,4,5-triyl triacetate (74) as a white solid (2.5 g, 65%). TLC: R_{f}=0.3 (petroleum ether/ethyl acetate=1:2). LCMS: Theoretical: C₃₀H₄₃N₃O₁₄ 669.27, Observed: m/z 670.30 [M+H]⁺. ¹H NMR (400 MHz, DMSO-d₆) δ (ppm): 6.84 (d, J=8.2 Hz, 1H), 6.72-6.63 (m, 1H), 6.59-6.45 (m, 1H), 5.53-5.39 (m, 2H), 5.17-5.01 (m, 2H), 4.86 (s, 2H), 4.72-4.63 (m, 3H), 3.65 (s, 3H), 3.38-3.31 (m, 2H), 2.90-2.69 (m, 6H), 2.04 (s, 3H), 2.00 (s, 6H), 1.36 (s, 9H).

To a solution of 1-(6-methyl-4-vinylpyridin-2-yl)-3-oxo-7,10,13,16,19,22,25,28-octaoxa-4-azahentriacontan-31-oic acid (24) (2.02 g, 3.29 mmol) in DMF (30 mL) was added HATU (1.70 g, 4.48 mmol), DIPEA (770.1 mg, 1038 μL, 5.97 mmol) and the mixture stirred at room temperature for 10 min, then 74 (2.0 g, 2.99 mmol) was added. The mixture was stirred at room temperature for 4 h under N₂ atmosphere. The mixture was diluted with water (100 mL) and extracted with ethyl acetate (20 mL×4). The combined organic layers were washed with brine (50 mL), dried over Na₂SO₄, filtered and concentrated. The residue was purified by column chromatography on silica gel (dichloromethane/methanol=100:1 to 40:1) to afford (2S,3S,4S,5R,6S)-2-(methoxycarbonyl)-6-(2-(1-(6-methyl-4-vinylpyridin-2-yl)-3-oxo-7,10,13,16,19,22,25,28-octaoxa-4-azahentriacontan-31-amido)-4-(4,7,10,10-tetramethyl-3,8-dioxo-2,9-dioxa-4,7-diazaundecyl)phenoxy)tetrahydro-2H-pyran-3,4,5-triyl triacetate (75) as a white solid (2.06 g, 54%). TLC: R_{f}=0.5 (dichloromethane/methanol=10:1). LCMS: Theoretical: C₆₀H₉₁N₅O₂₄ 1265.61, Observed: m/z 1266.65 [M+H]. ¹H NMR (400 MHz, DMSO-d₆) δ (ppm): 8.70 (s, 1H), 8.02-7.92 (m, 2H), 7.51-7.38 (m, 1H), 7.14-7.01 (m, 2H), 6.83-6.71 (m, 1H), 6.32-6.18 (m, 1H), 5.73-5.63 (m, 1H), 5.60 (d, J=7.8 Hz, 1H), 5.50 (t, J=9.6 Hz, 1H), 5.23-5.14 (m, 1H), 5.07 (t, J=9.6 Hz, 1H), 4.96 (s, 2H), 4.72 (d, J=10.0 Hz, 1H), 3.69 (t, J=6.2 Hz, 2H), 3.63 (s, 3H), 3.37 (t, J=5.8 Hz, 2H), 3.34-3.24 (m, 5H), 3.19 (q, J=5.6 Hz, 2H), 3.00 (t, J=7.2 Hz, 2H), 2.89-2.68 (m, 6H), 2.64-2.52 (m, 6H), 2.08-1.95 (m, 9H), 1.35 (s, 9H).

To a solution of 75 (1.2 g, 0.9 mmol) in acetonitrile (10.0 mL) at 0° C was added 4 N hydrochloric acid in 1,4-dioxane (10.0 mL). The mixture was stirred at 0° C for 1 h. The mixture was concentrated under vacuum and the residue was triturated with diethyl ether to afford (2S,3S,4S,5R,6S)-2-(Methoxycarbonyl)-6-(4-(((methyl(2-(methylamino)ethyl)carbamoyl)oxy)methyl)-2-(1-(6-methyl-4-vinylpyridin-2-yl)-3-oxo-7,10,13,16,19,22,25,28-octaoxa-4-azahentriacontan-31-amido)phenoxy)tetrahydro-2H-pyran-3,4,5-triyl triacetate (76) as a white solid (1.0 g, crude). LCMS: Theoretical: C₅₅H₈₃N₅O₂₂ 1165.55, Observed: m/z 584.40 [M+2H]²⁺.

To a solution of 15 (300 mg, 0.6 mmol) in DMF (10.0 mL) was added 76 (1.0 g, 0.9 mmol) and DIPEA (233 mg, 314 μL, 1.8 mmol). The mixture was stirred at room temperature overnight. The mixture was diluted with water (200 mL) and extracted with ethyl acetate (60 mL×3). The combined organic layers were washed with brine (60 mL), dried over Na₂SO₄, filtered and concentrated. The residue was purified by prep-TLC (dichloromethane/methanol=15:1) to afford (2S,3R,4S,5S,6S)-2-(4-((((2-(((((S)-3-(tert-butoxycarbonyl)-1-(chloromethyl)-2,3-dihydro-1H-benzo[e]indol-5-yl)oxy)carbonyl)(methyl)amino)ethyl)(methyl)carbamoyl)oxy)methyl)-2-(1-(6-methyl-4-vinylpyridin-2-yl)-3-oxo-7,10,13,16,19,22,25,28-octaoxa-4-azahentriacontan-31-amido)phenoxy)-6-(methoxycarbonyl)tetrahydro-2H-pyran-3,4,5-triyl triacetate (77) as a yellow solid (500 mg, 54%). LCMS: Theoretical: C₇₄H₁₀₁ClN₆O₂₆ 1524.65, Observed: m/z 763.80 [M+2H]²⁺.

To a solution of 77 (120 mg, 0.078 mmol) in acetonitrile (5 mL) at 0° C was added 4 N hydrochloric acid in 1,4-dioxane (5.0 mL). The mixture was stirred at 0° C for 1 h. The mixture was concentrated under vacuum and the residue was triturated with diethyl ether to afford (2S,3R,4S,5S,6S)-2-(4-((((2-(((((S)-1-(chloromethyl)-2,3-dihydro-1H-benzo[e]indol-5-yl)oxy)carbonyl)(methyl)amino)ethyl)(methyl)carbamoyl)oxy)methyl)-2-(1-(6-methyl-4-vinylpyridin-2-yl)-3-oxo-7,10,13,16,19,22,25,28-octaoxa-4-azahentriacontan-31-amido)phenoxy)-6-(methoxycarbonyl)tetrahydro-2H-pyran-3,4,5-triyl triacetate (78) as a red solid (100 mg, crude). LCMS: Theoretical: C₆₉H₉₃ClN₆O₂₄ 1424.59, Observed: m/z 714.55 [M+2H]²⁺.

To a solution of 17 (200 mg, 0.25 mmol) in dichloromethane (10 mL) was added dihydro-2H-pyran-2,6(3H)-dione (140.1 mg, 1.23 mmol), DIPEA (158.7 mg, 214 μL, 1.23 mmol) and DMAP (45.0 mg, 0.37 mmol.). The mixture was stirred at room temperature for 48 h. The mixture was diluted with dichloromethane (50 mL) and washed with water (20 mL) and brine (20 mL), dried over Na₂SO₄, filtered and concentrated. The residue was purified by prep-TLC (dichloromethane/methanol=15:1) to afford 5-((S)-1-(chloromethyl)-5-((methyl(2-(methyl(((4-(((2S,3R,4S,5S,6S)-3,4,5-triacetoxy-6-(methoxycarbonyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoic acid (79) as a yellow solid (50 mg, 22%). LCMS: Theoretical: C₄₄H₅₀ClN₃O₁₇ 927.28, Observed: m/z 950.55 [M+Na]⁺. ¹H NMR (400 MHz, DMSO-d₆) δ (ppm): 12.04 (s, 1H), 8.20 (s, 1H), 8.01-7.89 (m, 1H), 7.88-7.70 (m, 1H), 7.65-7.54 (m, 1H), 7.49-7.22 (m, 3H), 7.01-6.78 (m, 2H), 5.66-5.55 (m, 1H), 5.50-5.40 (m, 1H), 5.13-4.92 (m, 4H), 4.72-4.61 (m, 1H), 4.42-4.28 (m, 2H), 4.24-4.16 (m, 1H), 4.10-4.00 (m, 1H), 3.95-3.88 (m, 1H), 3.78-3.57 (m, 6H), 3.51-3.46 (m, 2H), 3.21-3.09 (m, 1H), 3.00-2.80 (m, 4H), 2.36-2.31 (m, 2H), 2.28-2.21 (m, 2H), 2.00 (d, J=5.4 Hz, 9H), 1.89-1.77 (m, 2H).

To a solution of 79 (50 mg, 0.054 mmol) and 78 (95 mg, 0.067 mmol) in dry DMA (5 mL) was added EDCl (42 mg, 0.271 mmol). The reaction mixture stirred at room temperature overnight. The mixture was diluted with water (100 mL) and extracted with ethyl acetate (50 mL×3). The combined organic layers were washed with brine (30 mL), dried over Na₂SO₄, filtered and concentrated. The residue was purified by reverse-phase column chromatography (acetonitrile/water=6:4) to afford (2S,3R,4S,5S,6S)-2-(4-((((2-(((((S)-1-(chloromethyl)-3-(5-((S)-1-(chloromethyl)-5-((methyl(2-(methyl(((3-(1-(6-methyl-4-vinylpyridin-2-yl)-3-oxo-7,10,13,16,19,22,25,28-octaoxa-4-azahentriacontan-31-amido)-4-(((2S,3R,4S,5S,6S)-3,4,5-triacetoxy-6-(methoxycarbonyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (80) as a yellowish solid (30 mg, 24%). LCMS: Theoretical: C₁₁₃H₁₄₁Cl₂N₉O₄₀ 2333.87, Observed: m/z 1169.35 [M+2H]²⁺.

To a solution of 80 (30 mg, 0.013 mmol) in acetonitrile (1 mL) and water (1 mL) at 0° C was added LiOH·H₂O (6.1 mg, 0.153 mmol). The mixture was stirred at 0° C for 2 h, and then acidified with 2 N hydrochloric acid to pH~3. After concentration, the residue was purified by prep-HPLC (eluent 5-80% acetonitrile in water containing 0.1% HCOOH) to afford (2S,3S,4S,5R,6S)-6-(4-((((2-(((((S)-3-(5-((S)-5-(((2-((((4-(((2S,3R,4S,5S,6S)-6-carboxy-3,4,5-trihydroxytetrahydro-2H-pyran-2-yl)oxy)-3-(1-(6-methyl-4-vinylpyridin-2-yl)-3-oxo-7,10,13,16,19,22,25,28-octaoxa-4-azahentriacontan-31-amido)benzyl)oxy)carbonyl)(methyl)amino)ethyl)(methyl)carbamoyl)oxy)-1-(chloromethyl)-1,2-dihydro-3H-benzo[e]indol-3-yl)-5-oxopentanoyl)-1-(chloromethyl)-2,3-dihydro-1H-benzo[e]indol-5-yl)oxy)carbonyl)(methyl)amino)ethyl)(methyl)carbamoyl)oxy)methyl)phenoxy)-3,4,5-trihydroxytetrahydro-2H-pyran-2-carboxylic acid (5) as a white solid (4.6 mg, 17%). LCMS: Theoretical: C₉₉H₁₂₅Cl₂N₉O₃₄ 2053.77, Observed: m/z 1028.95 [M+2H]²⁺.

### Preparation of Compound 6

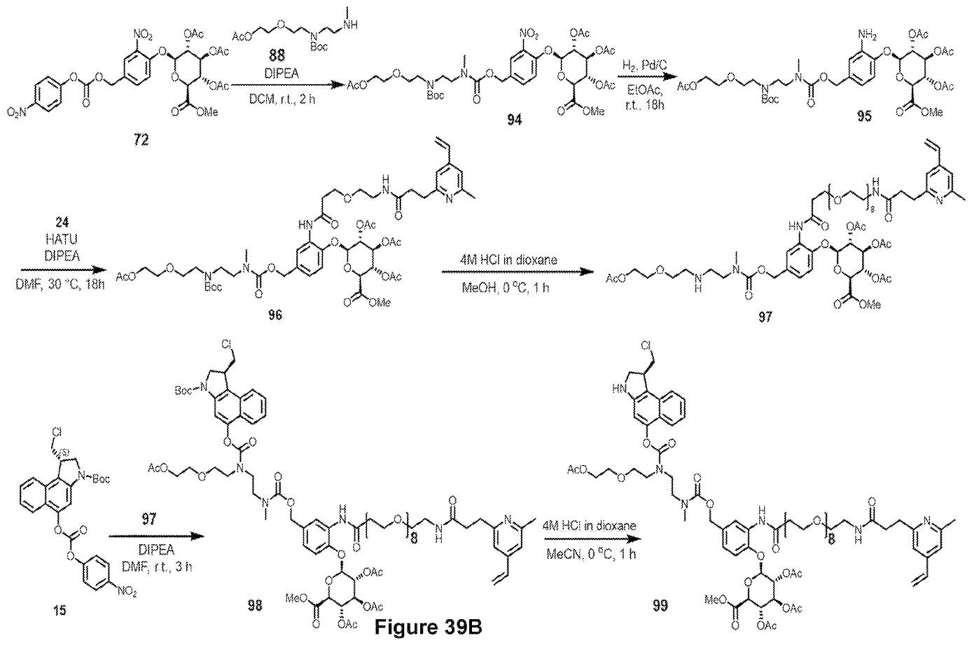

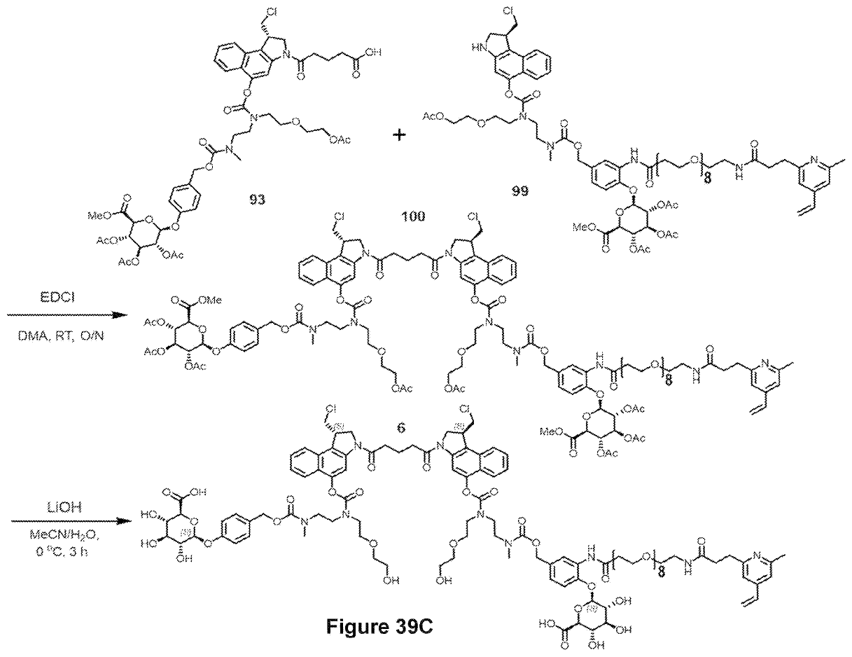

(2S,3S,4S,5R,6S)-6-(4-(7-((((S)-3-(5-((S)-5-(((2-((((4-(((2S,3R,4S,5S,6S)-6-carboxy-3,4,5-trihydroxytetrahydro-2H-pyran-2-yl)oxy)-3-(1-(6-methyl-4-vinylpyridin-2-yl)-3-oxo-7,10,13,16,19,22,25,28-octaoxa-4-azahentriacontan-31-amido)benzyl)oxy)carbonyl)(methyl)amino)ethyl)(2-(2-hydroxyethoxy)ethyl)carbamoyl)oxy)-1-(chloromethyl)-1,2-dihydro-3H-benzo[e]indol-3-yl)-5-oxopentanoyl)-1-(chloromethyl)-2,3-dihydro-1H-benzo[e]indol-5-yl)oxy)carbonyl)-12-hydroxy-4-methyl-3-oxo-2,10-dioxa-4,7-diazadodecyl)phenoxy)-3,4,5-trihydroxytetrahydro-2H-pyran-2-carboxylic acid (6) was prepared according to the schemes shown in FIGS. 39A to 39C.

To a solution of 2-(methylamino)ethan-1-ol (81) (50.0 g, 665.7 mmol) and N-(benzyloxycarbonyloxy)succinimide (165.9 g, 665.7 mmol) in THF (1.0 L) was added DIPEA (258.1 g, 2.0 mol). The mixture was stirred at room temperature for 1 h. The reaction mixture was diluted with aq. citric acid (2 L) and extracted with dichloromethane (200 mL×4). The combined organic layers were washed with aq. NaHCO₃ (200 mL) and brine (200 mL), dried over Na₂SO₄ and concentrated under vacuum. The residue was triturated with petroleum ether to afford benzyl (2-hydroxyethyl)(methyl)carbamate (82) as yellow oil (138.0 g, 99%). TLC: R_{f}=0.2 (petroleum ether/ethyl acetate=1:1). ¹H NMR (400 MHz, DMSO-d₆) δ (ppm): 7.39-7.27 (m, 5H), 5.08 (s, 2H), 4.82-4.68 (m, 1H), 3.59-3.51 (m, 2H), 3.32 (t, J=6.0 Hz, 2H), 2.92 (d, J=7.6 Hz, 3H).

To a solution of 82 (80.0 g, 0.38 mol) in dichloromethane (1000 mL) was added DMSO (298.9 g, 3.18 mol), DIPEA (247.1 g, 333.0 mL, 1.91 mol) and pyridine sulfur trioxide (304.2 g, 1.91 mol). The mixture was stirred at −10° C for 2 h. The reaction mixture was diluted with 1N hydrochloric acid (2 L) and extracted with dichloromethane (300 mL×3). The combined organic layers were washed with aq. NaHCO₃ (150 mL) and brine (100 mL), dried over Na₂SO₄ and concentrated under vacuum. The residue was purified by column chromatography on silica gel (eluent: petroleum ether/ethyl acetate=10:1 to 3:1) to afford benzyl methyl(2-oxoethyl)carbamate (83) as yellow oil (60 g, 76%). TLC: R_{f}=0.5 (petroleum ether/ethyl acetate=1:1). LCMS: Theoretical: C₁₁H₁₃NO₃ 207.09, Observed: m/z 208.10 [M+H]⁺. ¹H NMR (400 MHz, DMSO-d₆) δ (ppm): 9.51 (s, 1H), 7.51-7.23 (m, 5H), 5.20-5.01 (m, 2H), 4.17 (d, J=21.0 Hz, 2H), 2.89 (d, J=18.0 Hz, 3H).

To a solution of 2-(2-aminoethoxy)ethan-1-ol (84) (20.00 g, 190 mmol) in dichloromethane (300 mL) was added 83 (35.4 g, 171 mmol). The mixture was stirred at room temperature overnight. After cooling to 0° C, NaBH(OAc)₃ (36.0 g, 170 mmol) was added and the mixture was warmed to room temperature and stirred for 3 h. To the crude reaction mixture of benzyl (2-((2-(2-hydroxyethoxy)ethyl)amino)ethyl)(methyl)carbamate (85) was added di-tert-butyl dicarbonate (38.3 g, 175 mmol) and DIPEA (49 g, 66 mL, 380 mmol). The mixture was stirred at room temperature for 5 h. The mixture was diluted with dichloromethane (300 mL), washed with water (100 mL) and brine (100 mL). The organic layer was dried over Na₂SO₄ and concentrated under vacuum. The residue was purified by column chromatography on silica gel (eluent petroleum ether/ethyl acetate=2:1) to afford benzyl (2-((tert-butoxycarbonyl)(2-(2-hydroxyethoxy)ethyl)amino)ethyl)(methyl)carbamate (86) as a yellow oil (25.6 g, 38%). TLC: R_{f}=0.3 (petroleum ether/ethyl acetate=1:2). LCMS: Theoretical: C₂₀H₃₂N₂O₆ 396.23, Observed: m/z 397.15 [M+H]⁺. ¹H NMR (400 MHz, Chloroform-d) δ (ppm): 7.38-7.27 (m, 5H), 5.10 (s, 2H), 3.76-3.70 (m, 2H), 3.59-3.50 (m, 5H), 3.43-3.28 (m, 5H), 2.98-2.91 (m, 3H), 1.43 (s, 9H).

To solution of 86 (30.0 g, 75.7 mmol) in dichloromethane (300 mL) was added acetic anhydride (10.2 g, 9.4 mL, 99.9 mmol), TEA (15.22 g, 21.0 mL, 150.4 mmol) and DMAP (923 mg, 7.57 mmol). The mixture was stirred at room temperature for 5 h. The mixture was diluted with dichloromethane (150 mL), washed with water (100 mL) and brine (100 mL). The organic layer was dried over Na₂SO₄ and concentrated under vacuum. The residue was purified by column chromatography on silica gel (eluent petroleum ether/ethyl acetate=5:1) to afford 7-(tert-butoxycarbonyl)-4-methyl-3-oxo-1-phenyl-2,10-dioxa-4,7-diazadodecan-12-yl acetate (87) as a yellow oil (8.0 g, 24%). TLC: R_{f}=0.3 (petroleum ether/ethyl acetate=1:2). LCMS: Theoretical: C₂₂H₃₄N₂O₇ 438.24, Observed: m/z 439.2 [M+H]⁺. ¹H NMR (400 MHz, Chloroform-d) δ (ppm): 7.40-7.27 (m, 5H), 5.11 (s, 2H), 4.24-4.14 (m, 2H), 3.65-3.50 (m, 4H), 3.43-3.23 (m, 6H), 3.01-2.90 (m, 3H), 2.03 (s, 3H), 1.43 (s, 9H).

To a solution of 87 (22.0 g, 50 mmol) in acetic acid (3.0 mL) and methanol (300.0 mL) was added 10% Pd/C (2.0 g). After stirring at 50° C overnight under H₂ atmosphere, the mixture was filtered. The filtrate was concentrated under vacuum to afford 2-(2-((tert-butoxycarbonyl)(2-(methylamino)ethyl)amino)ethoxy)ethyl acetate (88) as a colorless oil (20 g, crude). LCMS: Theoretical C₁₄H₂₈N₂O₅ 304.20, Observed: m/z=305.16 [M+H]⁺. ¹H NMR (400 MHz, Chloroform-d) δ (ppm): 4.21 (dq, J=5.0, 2.4 Hz, 2H), 3.68-3.30 (m, 8H), 3.12 (s, 1H), 2.64 (s, 2H), 2.07 (s, 3H), 2.01 (s, 4H), 1.44 (s, 9H).

To a solution of 88 (12.0 g, 39.6 mmol) in dichloromethane (200 mL) was added DIPEA (5.1 g, 6.87 mL, 39.6 mmol) and DMAP (644 mg. 5.28 mmol). The mixture was stirred at room temperature for 3 h. The mixture was poured into water (200 mL) and extracted with dichloromethane (200 mL×3). The combined organic layers were dried over Na₂SO₄ and concentrated under vacuum. The residue was purified by column chromatography on silica gel (eluent: petroleum ether/ethyl acetate/dichloromethane=5:1:1) and reverse-phase column chromatography (eluent: acetonitrile/water (0.1% HCOOH)=3:1) to afford (2S,3R,4S,5S,6S)-2-(4-(7-(tert-butoxycarbonyl)-4-methyl-3,14-dioxo-2,10,13-trioxa-4,7-diazapentadecyl)phenoxy)-6-(methoxycarbonyl)tetrahydro-2H-pyran-3,4,5-triyl triacetate (89) as a light-yellow solid (9.8 g, 48%). TLC: R_{f}=0.3 (dichloromethane/methanol=20:1). LCMS: Theoretical: C₃₅H₅₀N₂O₁₇ 770.31, Observed: m/z 771.25 [M+H]⁺. ¹H NMR (400 MHz, DMSO-d₆) δ (ppm): 7.32 (d, J=8.4 Hz, 2H), 6.98 (d, J=8.4 Hz, 2H), 5.65 (d, J=8.0 Hz, 1H), 5.47 (t, J=9.6 Hz, 1H), 5.13-5.03 (m, 2H), 4.98 (s, 2H), 4.70 (d, J=10.0 Hz, 1H), 4.09 (s, 2H), 3.63 (s, 3H), 3.61-3.32 (m, 7H), 3.30-3.16 (m, 3H), 2.84 (d, J=7.0 Hz, 3H), 2.04-1.97 (m, 12H), 1.35 (s, 9H).

To a solution of 89 (3.0 g, 3.89 mmol) in acetonitrile (20 mL) at 0° C was added 4 N hydrochloric acid/1,4-dioxane (20.0 mL). The mixture was stirred at 0° C for 1 h and concentrated under vacuum. The residue was triturated with diethyl ether to afford (2S,3S,4S,5R,6S)-2-(Methoxycarbonyl)-6-(4-(4-methyl-3,14-dioxo-2,10,13-trioxa-4,7-diazapentadecyl)phenoxy)tetrahydro-2H-pyran-3,4,5-triyl triacetate (90) as a white solid (2.6 g, crude), which was used in next step without further purification. LCMS: Theoretical: C₃₀H₄₂N₂O₁₅ 670.26, Observed: m/z=671.20 [M+H]⁺.

The crude residue of 15 (298 mg, 599 μmol) was dissolved into DMF (5 mL), then 90 (602 mg, 898 μmol) and DIPEA (231 mg, 1.79 mmol) were added. The mixture was stirred at room temperature for 3 h. The mixture was diluted with ethyl acetate (50 mL), washed with water (20 mL) and brine (20 mL), dried over Na₂SO₄, filtered and concentrated. The residue was purified by column chromatography (eluent petroleum ether/ethyl acetate=1:1) and reverse-phase column chromatography (eluent acetonitrile/water=7:3) to afford (2S,3R,4S,5S,6S)-2-(4-(7-((((S)-3-(tert-Butoxycarbonyl)-1-(chloromethyl)-2,3-dihydro-1H-benzo[e]indol-5-yl)oxy)carbonyl)-4-methyl-3,14-dioxo-2,10,13-trioxa-4,7-diazapentadecyl)phenoxy)-6-(methoxycarbonyl)tetrahydro-2H-pyran-3,4,5-triyl triacetate (91) as a light-yellow oil (200 mg, 22%). LCMS: Theoretical: C₄₉H₆₀ClN₃O₁₉ 1029.35, Observed: m/z 1052.55 [M+Na]⁺. ¹H NMR (400 MHz, DMSO-d₆) δ (ppm): 7.96-7.89 (m, 1H), 7.88-7.77 (m, 1H), 7.59-7.52 (m, 1H), 7.42-7.34 (m, 1H), 7.32-6.91 (m, 3H), 5.66-5.54 (m, 1H), 5.48-5.41 (m, 1H), 5.11-4.92 (m, 4H), 4.72-4.62 (m, 1H), 4.29-4.07 (m, 4H), 3.90 (s, 1H), 3.80-3.36 (m, 14H), 2.94-2.83 (m, 3H), 2.03-1.95 (m, 12H), 1.51 (s, 9H).

To a solution of 91 (300 mg, 291 μmol) in acetonitrile (5 mL) at 0° C was added 4 N hydrochloric acid/1,4-dioxane (5 mL). The mixture was stirred at 0° C for 1 h and concentrated under vacuum. The residue was triturated with diethyl ether to afford (2S,3R,4S,5S,6S)-2-(4-(7-((((S)-1-(chloromethyl)-2,3-dihydro-1H-benzo[e]indol-5-yl)oxy)carbonyl)-4-methyl-3,14-dioxo-2,10,13-trioxa-4,7-diazapentadecyl)phenoxy)-6-(methoxycarbonyl)tetrahydro-2H-pyran-3,4,5-triyl triacetate (92) as a white solid (300 mg, crude), which was used to next step directly. LCMS: Theoretical: C₄₄H₅₂ClN₃O₁₇ 929.30, Observed: m/z=930.20 [M+H]⁺.

To a solution of 92 (300 mg crude, 291 μmol) and dihydro-2H-pyran-2,6(3H)-dione (184 mg, 1.61 mmol) in dichloromethane (5.0 mL) was added DIPEA (124 mg, 168 μL, 966 μmol) and DMAP (19.6 mg, 161 μmol). The mixture was stirred at room temperature overnight. The mixture was diluted with dichloromethane (50 mL), washed with water (20 mL) and brine (20 mL), dried over Na₂SO₄, filtered and concentrated. The residue was purified by prep-TLC (dichloromethane/methanol=15:1) to afford 5-((S)-5-(((2-(2-Acetoxyethoxy)ethyl)(2-(methyl(((4-(((2S,3R,4S,5S,6S)-3,4,5-triacetoxy-6-(methoxycarbonyl)tetrahydro-2H-pyran-2-yl)oxy)benzyl)oxy)carbonyl)amino)ethyl)carbamoyl)oxy)-1-(chloromethyl)-1,2-dihydro-3H-benzo[e]indol-3-yl)-5-oxopentanoic acid (93) as a light-yellow oil (150 mg, 49%). LCMS: Theoretical: C₄₉H₅₈ClN₃O₂₀ 1043.33, Observed: m/z 1044.60 [M+H]⁺. ¹H NMR (400 MHz, DMSO-d₆) δ (ppm): 8.39-8.26 (m, 1H), 7.95-7.68 (m, 2H), 7.52 (s, 1H), 7.43-7.27 (m, 3H), 7.04-6.89 (m, 2H), 5.40-5.24 (m, 3H), 5.19-4.99 (m, 3H), 4.37-4.08 (m, 6H), 4.00-3.91 (m, 1H), 3.80 (s, 1H), 3.77-3.46 (m, 14H), 3.03-2.91 (m, 3H), 2.75-2.35 (m, 5H), 2.06-2.02 (m, 12H).

To a solution of 88 (4.2 g, 13.8 mmol) and DIPEA (2.4 g, 3.23 mL, 18.4 mmol). The mixture was stirred at room temperature for 2 h. The reaction mixture was diluted with water (100 mL) and extracted with ethyl acetate (20 mL×4). The combined organic layers were washed with brine (100 mL), dried over Na₂SO₄, filtered and concentrated. The residue was purified by column chromatography on silica gel (eluent: petroleum ether/ethyl acetate=3:1 to 1:1) to afford (2S,3R,4S,5S,6S)-2-(4-(7-(tert-butoxycarbonyl)-4-methyl-3,14-dioxo-2,10,13-trioxa-4,7-diazapentadecyl)-2-nitrophenoxy)-6-(methoxycarbonyl)tetrahydro-2H-pyran-3,4,5-triyl triacetate (94) as a yellowish oil (5.0 g, 66%). TLC: R_{f}=0.4 (petroleum ether/ethyl acetate=1:2). LCMS: Theoretical: C₃₅H₄₉N₃O₁₉ 815.30, Observed: m/z 816.25 [M+H]⁺. ¹H NMR (400 MHz, DMSO-d₆) δ (ppm): 7.88 (s, 1H), 7.73-7.64 (m, 1H), 7.47-7.33 (m, 1H), 5.79-5.73 (m, 1H), 5.46 (t, J=9.4 Hz, 1H), 5.15-5.01 (m, 4H), 4.74 (d, J=9.8 Hz, 1H), 4.13-4.03 (m, 2H), 3.64 (s, 3H), 3.59-3.41 (m, 4H), 3.39-3.33 (m, 2H), 3.31-3.15 (m, 4H), 2.91-2.79 (m, 3H), 2.05-1.94 (m, 12H), 1.35 (s, 9H).

To solution of 94 (4.5 g, 5.5 mmol) in ethyl acetate (30 mL) was added 10% Pd/C (900 mg). The reaction mixture was stirred under a H₂ atmosphere for 24 h. The mixture was filtered and concentrated. The residue obtained was purified by column chromatography on silica gel (eluent petroleum ether/ethyl acetate=5:1 to 1:1) to afford (2S,3R,4S,5S,6S)-2-(2-amino-4-(7-(tert-butoxycarbonyl)-4-methyl-3,14-dioxo-2,10,13-trioxa-4,7-diazapentadecyl)phenoxy)-6-(methoxycarbonyl)tetrahydro-2H-pyran-3,4,5-triyl triacetate (95) as a white solid (3.5 g, 81%). TLC: R_{f}=0.3 (petroleum ether/ethyl acetate=1:2). LCMS: Theoretical: C₃₅H₅₁N₃O₁₇ 785.32, Observed: m/z 786.25 [M+H]. ¹H NMR (400 MHz, DMSO-d₆) δ (ppm): 6.87-6.81 (m, 1H), 6.66 (m, 1H), 6.59-6.45 (m, 1H), 5.54-5.38 (m, 2H), 5.16-5.01 (m, 2H), 4.86 (s, 2H), 4.74-4.61 (m, 3H), 4.15-4.04 (m, 2H), 3.64 (s, 3H), 3.60-3.40 (m, 4H), 3.31-3.19 (m, 4H), 2.84 (d, J=8.6 Hz, 3H), 2.03 (s, 3H), 2.02-1.97 (m, 9H), 1.37 (s, 9H).

To a solution of 24 (3.0 g, 4.90 mmol) in DMF (40 mL) was added HATU (2.54 g, 6.68 mmol), DIPEA (1.15 g, 1.55 mL, 8.91 mmol) and the mixture stirred at room temperature for 10 min, then (2S,3R,4S,5S,6S)-2-(2-amino-4-(7-(tert-butoxycarbonyl)-4-methyl-3,14-dioxo-2,10,13-trioxa-4,7-diazapentadecyl)phenoxy)-6-(methoxycarbonyl)tetrahydro-2H-pyran-3,4,5-triyl triacetate (95) (3.5 g, 4.45 mmol) was added and the mixture was stirred at 30° C overnight under N₂ atmosphere. The mixture was diluted with water (100 mL) and extracted with ethyl acetate (20 mL×4). The combined organic layers were washed with brine (100 mL), dried over Na₂SO₄, filtered and concentrated. The residue was purified by reverse-phase column chromatography (eluent acetonitrile/water=11:9) to afford (2S,3R,4S,5S,6S)-2-(4-(7-(tert-butoxycarbonyl)-4-methyl-3,14-dioxo-2,10,13-trioxa-4,7-diazapentadecyl)-2-(1-(6-methyl-4-vinylpyridin-2-yl)-3-oxo-7,10,13,16,19,22,25,28-octaoxa-4-azahentriacontan-31-amido)phenoxy)-6-(methoxycarbonyl)tetrahydro-2H-pyran-3,4,5-triyl triacetate (96) as a white solid (2.03 g, 33%). TLC: R, =0.5 (dichloromethane/methanol=10:1). LCMS: Theoretical: C₆₅H₉₉N₅O₂₇ 1381.65, Observed: m/z 1382.55 [M+H]. ¹H NMR (400 MHz, DMSO-d₆) δ (ppm): 8.69 (s, 1H), 8.10-7.92 (m, 2H), 7.82 (d, J=11.2 Hz, 2H), 7.18-7.00 (m, 2H), 6.88 (dd, J=17.6, 10.8 Hz, 1H), 6.47 (d, J=17.6 Hz, 1H), 5.91 (d, J=10.8 Hz, 1H), 5.60 (d, J=7.8 Hz, 1H), 5.50 (t, J=9.6 Hz, 1H), 5.25-5.14 (m, 1H), 5.07 (t, J=9.6 Hz, 1H), 4.96 (s, 2H), 4.72 (d, J=10.0 Hz, 1H), 4.14-4.02 (m, 2H), 3.70 (t, J=6.4 Hz, 2H), 3.64 (s, 3H), 3.61-3.39 (m, 33H), 3.37 (t, J=5.6 Hz, 2H), 3.35-3.10 (m, 10H), 2.84 (d, J=11.2 Hz, 3H), 2.69-2.64 (m, 4H), 2.63-2.54 (m, 2H), 2.08-1.91 (m, 12H), 1.36 (s, 9H).

To a solution of 96 (1.0 g, 724 μmol) in acetonitrile (20 mL) at 0° C was added 4 N hydrochloric acid/1,4-dioxane (20 mL). The mixture was stirred at 0° C for 1 h and concentrated under vacuum. The residue was triturated with diethyl ether to afford (2S,3S,4S,5R,6S)-2-(methoxycarbonyl)-6-(4-(4-methyl-3,14-dioxo-2,10,13-trioxa-4,7-diazapentadecyl)-2-(1-(6-methyl-4-vinylpyridin-2-yl)-3-oxo-7,10,13,16,19,22,25,28-octaoxa-4-azahentriacontan-31-amido)phenoxy)tetrahydro-2H-pyran-3,4,5-triyl triacetate (97) as a white solid (1.0 g, crude). LCMS: Theoretical: C₆₀H₉₁N₅O₂₅ 1281.30, Observed: m/z=642.20 [M+2H]²⁺.

97 (574 mg, 448 μmol) and DIPEA (115 mg, 156 μL, 897 mmol) were dissolved in DMF (5.0 mL) and added to crude mixture of 15 (148 mg, 299 μmol) in DMF (2.0 mL).

The mixture was stirred at room temperature for 3 h. The mixture was diluted with ethyl acetate (50 mL), washed with water (20 mL) and brine (20 mL), dried over Na₂SO₄, filtered and concentrated. The residue was purified by column chromatography on silica gel (eluent petroleum ether/ethyl acetate=1:1) to afford (2S,3R,4S,5S,6S)-2-(4-(7-((((S)-3-(tert-butoxycarbonyl)-1-(chloromethyl)-2,3-dihydro-1H-benzo[e]indol-5-yl)oxy)carbonyl)-4-methyl-3,14-dioxo-2,10,13-trioxa-4,7-diazapentadecyl)-2-(1-(6-methyl-4-vinylpyridin-2-yl)-3-oxo-7,10,13,16,19,22,25,28-octaoxa-4-azahentriacontan-31-amido)phenoxy)-6-(methoxycarbonyl)tetrahydro-2H-pyran-3,4,5-triyl triacetate (98) (150 mg, 30%) as a light-yellow oil. LCMS: Theoretical: C₇₉H₁₀₉ClN₆O₂₉ 1640.69, Observed: m/z 1641.95 [M+H]. ¹H NMR (400 MHz, DMSO-d₆) δ (ppm): 8.73-8.60 (m, 1H), 8.01-7.74 (m, 4H), 7.59-7.47 (m, 1H), 7.43-7.31 (m, 1H), 7.13 (d, J=64.1 Hz, 4H), 6.68 (dd, J=17.6, 10.9 Hz, 1H), 6.11 (d, J=17.7 Hz, 1H), 5.64-5.42 (m, 3H), 5.24-4.90 (m, 4H), 4.74-4.65 (m, 1H), 4.28-3.99 (m, 5H), 3.94-3.85 (m, 1H), 3.78-3.55 (m, 14H), 3.54-3.43 (m, 36H), 3.22-3.15 (m, 3H), 2.95-2.84 (m, 5H), 2.61-2.55 (m, 2H), 2.45 (s, 3H), 2.05-1.96 (m, 12H), 1.56-1.44 (m, 8H).

To a solution of 98 (300 mg, 183 μmol) in acetonitrile (5 mL) at 0° C was added 4 N hydrochloric acid/1,4-dioxane (5 mL). The mixture was stirred at 0° C for 1 h and concentrated under vacuum. The residue was triturated with diethyl ether to afford (2S,3R,4S,5S,6S)-2-(4-(7-((((S)-1-(chloromethyl)-2,3-dihydro-1H-benzo[e]indol-5-yl)oxy)carbonyl)-4-methyl-3,14-dioxo-2,10,13-trioxa-4,7-diazapentadecyl)-2-(1-(6-methyl-4-vinylpyridin-2-yl)-3-oxo-7,10,13,16,19,22,25,28-octaoxa-4-azahentriacontan-31-amido)phenoxy)-6-(methoxycarbonyl)tetrahydro-2H-pyran-3,4,5-triyl triacetate (99) (300 mg, crude) as a white solid, which was used directly in the next step without further purification. LCMS: Theoretical: C₇₄H₁₀₁ClN₆O₂₇ 1540.64, Observed: m/z 772.20 [M+2H]²⁺.

To a solution of 93 (70 mg, 67.1 μmol) and 99 (154 mg, 100 μmol) in DMA (2.0 mL) was added EDCl (63 mg, 406 μmol). The mixture was stirred at room temperature overnight. The mixture was diluted with ethyl acetate (50 mL), washed with water (20 mL) and brine (20 mL), dried over Na₂SO₄, filtered and concentrated. The residue was purified by prep-TLC (dichloromethane/methanol=15:1) and prep-HPLC (eluent 5-80% acetonitrile in water containing 0.1% HCOOH) to afford (2S,3R,4S,5S,6S)-2-(4-(7-((((S)-3-(5-((S)-5-(((2-(2-acetoxyethoxy)ethyl)(2-(methyl(((3-(1-(6-methyl-4-vinylpyridin-2-yl)-3-oxo-7,10,13,16,19,22,25,28-octaoxa-4-azahentriacontan-31-amido)-4-(((2S,3R,4S,5S,6S)-3,4,5-triacetoxy-6-(methoxycarbonyl)tetrahydro-2H-pyran-2-yl)oxy)benzyl)oxy)carbonyl)amino)ethyl)carbamoyl)oxy)-1-(chloromethyl)-1,2-dihydro-3H-benzo[e]indol-3-yl)-5-oxopentanoyl)-1-(chloromethyl)-2,3-dihydro-1H-benzo[e]indol-5-yl)oxy)carbonyl)-4-methyl-3,14-dioxo-2,10,13-trioxa-4,7-diazapentadecyl)phenoxy)-6-(methoxycarbonyl)tetrahydro-2H-pyran-3,4,5-triyl triacetate (100) as a white solid (20 mg, 1%). LCMS: Theoretical: C₁₂₃H₁₅₇Cl₂N₉O₄₆ 2565.96, Observed: m/z 1285.10 [M+2H]². ¹H NMR (400 MHz, DMSO-d₆) δ (ppm): 8.73-8.45 (m, 2H), 8.26-8.20 (m, 2H), 8.05-7.75 (m, 7H), 7.62-7.52 (m, 2H), 7.41 (q, J=7.6 Hz, 3H), 7.29 (s, 1H), 7.22-6.91 (m, 6H), 6.65 (dd, J=17.8, 10.8 Hz, 1H), 6.04 (d, J=17.6 Hz, 1H), 5.60 (dd, J=19.0, 7.8 Hz, 2H), 5.51-5.42 (m, 3H), 5.22-5.15 (m, 1H), 5.11-4.93 (m, 8H), 4.73-4.63 (m, 2H), 4.47-3.84 (m, 17H), 3.75-3.60 (m, 22H), 3.51-3.44 (m, 34H), 3.21-3.15 (m, 2H), 2.97-2.82 (m, 9H), 2.60-2.55 (m, 3H), 2.48-2.44 (m, 2H), 2.41 (s, 2H), 2.03-1.96 (m, 24H).

To a solution of 100 (20 mg, 7.8 μmol) in acetonitrile (1.0 mL) was added a solution of LiOH·H₂O (13 mg, 312 μmol) in water (1 mL) at 0 °C. The mixture was stirred at 0° C for 3 h. The pH was adjusted with 3 N hydrochloric acid to pH 2-3 and was purified by prep-HPLC (eluent: 45% acetonitrile in water) to afford (2S,3S,4S,5R,6S)-6-(4-(7-((((S)-3-(5-((S)-5-(((2-((((4-(((2S,3R,4S,5S,6S)-6-carboxy-3,4,5-trihydroxytetrahydro-2H-pyran-2-yl)oxy)-3-(1-(6-methyl-4-vinylpyridin-2-yl)-3-oxo-7,10,13,16,19,22,25,28-octaoxa-4-azahentriacontan-31-amido)benzyl)oxy)carbonyl)(methyl)amino)ethyl)(2-(2-hydroxyethoxy)ethyl)carbamoyl)oxy)-1-(chloromethyl)-1,2-dihydro-3H-benzo[e]indol-3-yl)-5-oxopentanoyl)-1-(chloromethyl)-2,3-dihydro-1H-benzo[e]indol-5-yl)oxy)carbonyl)-12-hydroxy-4-methyl-3-oxo-2,10-dioxa-4,7-diazadodecyl)phenoxy)-3,4,5-trihydroxytetrahydro-2H-pyran-2-carboxylic acid (6) as a white solid (4.7 mg, 27%). LCMS: Theoretical: C₁₀₅H₁₃₇Cl₂N₉O₃₈ 2201.84, Observed: m/z 1102.15 [M+2H]²⁺. ¹H NMR (400 MHz, DMSO-d₆) δ (ppm): 9.21 (s, 1H), 8.27-8.17 (m, 4H), 7.98-7.89 (m, 4H), 7.88-7.79 (m, 2H), 7.61-7.48 (m, 3H), 7.45-7.32 (m, 3H), 7.26-7.22 (m, 1H), 7.18-7.07 (m, 4H), 6.97 (t, J=8.0 Hz, 2H), 6.05 (d, J=17.8 Hz, 1H), 5.79 (br s, 1H), 5.46 (d, J=10.8 Hz, 1H), 5.11-4.86 (m, 7H), 4.48-4.17 (m, 8H), 4.10-3.99 (m, 3H), 3.96-3.87 (m, 3H), 3.80-3.59 (m, 21H), 3.58-3.50 (m, 21H), 3.37 (t, J=5.8 Hz, 8H), 3.31-3.22 (m, 13H), 3.21-3.15 (m, 5H), 2.97-2.83 (m, 12H), 2.71-2.59 (m, 6H), 2.41 (s, 3H), 1.95 (br s, 3H).

### Preparation of Compound 7

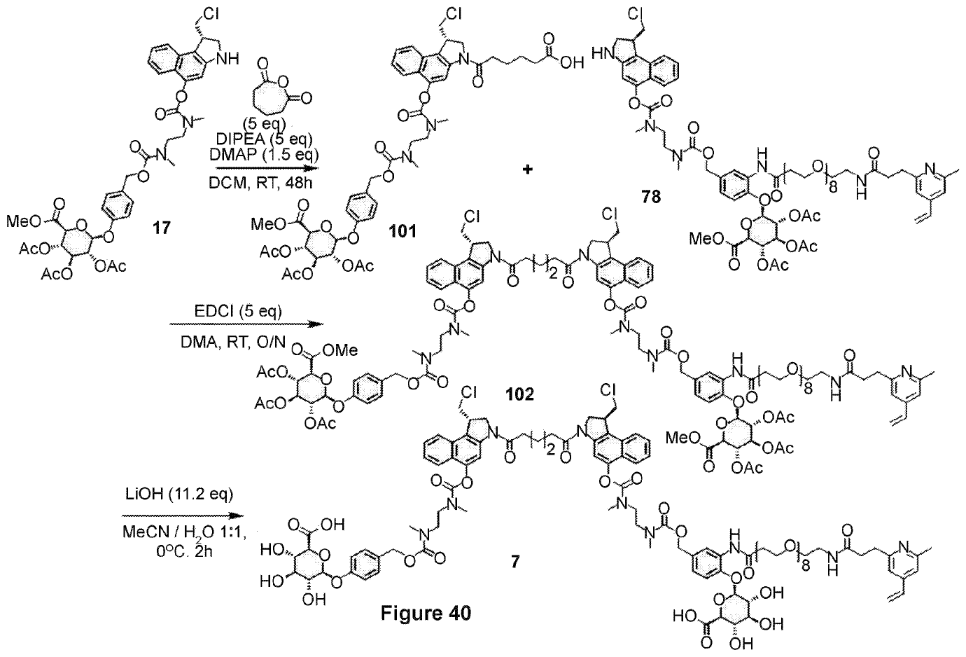

(2S,3S,4S,5R,6S)-6-(4-((((2-(((((S)-3-(6-((S)-5-(((2-((((4-(((2S,3R,4S,5S,6S)-6-carboxy-3,4,5-trihydroxytetrahydro-2H-pyran-2-yl)oxy)-3-(1-(6-methyl-4-vinylpyridin-2-yl)-3-oxo-7,10,13,16,19,22,25,28-octaoxa-4-azahentriacontan-31-amido)benzyl)oxy)carbonyl)(methyl)amino)ethyl)(methyl)carbamoyl)oxy)-1-(chloromethyl)-1,2-dihydro-3H-benzo[e]indol-3-yl)-6-oxohexanoyl)-1-(chloromethyl)-2,3-dihydro-1H-benzo[e]indol-5-yl)oxy)carbonyl)(methyl)amino)ethyl)(methyl)carbamoyl)oxy)methyl)phenoxy)-3,4,5-trihydroxytetrahydro-2H-pyran-2-carboxylic acid (7) was prepared according to the scheme shown in FIG. 40.

A solution of 17 (280 mg, 0.34 mmol) in dichloromethane (10 mL) was added oxepane-2,7-dione (220.3 mg, 1.72 mmol), DIPEA (221.9 mg, 298 μL, 1.72 mmol) and DMAP (66.5 mg, 0.52 mmol). The mixture was stirred at room temperature for 48 h. The mixture was diluted with dichloromethane (50 mL) and washed with water (20 mL) and brine (20 mL), dried over Na₂SO₄, filtered and concentrated. The residue was purified by prep-TLC (dichloromethane/methanol=15:1) to afford 6-((S)-1-(chloromethyl)-5-((methyl(2-(methyl(((4-(((2S,3R,4S,5S,6S)-3,4,5-triacetoxy-6-(methoxycarbonyl)tetrahydro-2H-pyran-2-yl)oxy)benzyl)oxy)carbonyl)amino)ethyl)carbamoyl)oxy)-1,2-dihydro-3H-benzo[e]indol-3-yl)-6-oxohexanoic acid (101) as a yellowish solid (130 mg, 40%). LCMS: Theoretical: C₄₅H₅₂ClN₃O₁₇ 941.30, Observed: m/z 964.55 [M+Na]⁺. ¹H NMR (400 MHz, DMSO-d₆) δ (ppm): 12.00 (s, 1H), 8.20 (s, 1H), 8.00-7.91 (m, 1H), 7.88-7.71 (m, 1H), 7.61-7.54 (m, 1H), 7.47-7.19 (m, 3H), 7.03-6.78 (m, 2H), 5.66-5.56 (m, 1H), 5.49-5.37 (m, 1H), 5.11-4.96 (m, 4H), 4.77-4.62 (m, 1H), 4.45-4.15 (m, 3H), 4.11-3.98 (m, 1H), 3.96-3.86 (m, 1H), 3.84-3.66 (m, 1H), 3.65-3.57 (m, 4H), 3.53-3.44 (m, 2H), 3.21-3.09 (m, 1H), 3.02-2.82 (m, 5H), 2.68-2.51 (m, 2H), 2.30-2.22 (m, 2H), 2.00 (d, J=5.4 Hz, 9H), 1.61 (s, 4H).

To a solution of 101 (100 mg, 0.11 mmol) and 78 (226.9 mg, 0.16 mmol) in dry DMA (5 mL) was added EDCl (81.4 mg, 0.52 mmol). The reaction mixture stirred at room temperature overnight. The mixture was diluted with water (100 mL) and extracted with ethyl acetate (50 mL×3). The combined organic layers were washed with brine (30 mL), dried over Na₂SO₄, filtered and concentrated. The residue was purified by reverse-phase column chromatography (eluent acetonitrile/water=2:3) to afford (2S,3R,4S,5S,6S)-2-(4-((((2-(((((S)-1-(chloromethyl)-3-(6-((S)-1-(chloromethyl)-5-((methyl(2-(methyl(((3-(1-(6-methyl-4-vinylpyridin-2-yl)-3-oxo-7,10,13,16,19,22,25,28-octaoxa-4-azahentriacontan-31-amido)-4-(((2S,3R,4S,5S,6S)-3,4,5-triacetoxy-6-(methoxycarbonyl)tetrahydro-2H-pyran-2-yl)oxy)benzyl)oxy)carbonyl)amino)ethyl)carbamoyl)oxy)-1,2-dihydro-3H-benzo[e]indol-3-yl)-6-oxohexanoyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (102) as a yellowish solid (60 mg, 24%). LCMS: Theoretical: C₁₁₄C₁₁₄₃Cl₂N₉O₄₀ 2347.88, Observed: m/z 1175.60 [M+2H]²⁺.

To a solution of 102 (60 mg, 0.026 mmol) in acetonitrile (1 mL) and water (1 mL) at 0° C was added LiOH·H₂O (12.2 mg, 0.291 mmol). The mixture was stirred at 0° C for 2 h, then acidified with 2 N hydrochloric acid to pH~3. After concentration, the residue was purified by prep-HPLC (eluent 5-80% acetonitrile in water containing 0.1% HCOOH) to afford (2S,3S,4S,5R,6S)-6-(4-((((2-(((((S)-3-(6-((S)-5-(((2-((((4-(((2S,3R,4S,5S,6S)-6-carboxy-3,4,5-trihydroxytetrahydro-2H-pyran-2-yl)oxy)-3-(1-(6-methyl-4-vinylpyridin-2-yl)-3-oxo-7,10,13,16,19,22,25,28-octaoxa-4-azahentriacontan-31-amido)benzyl)oxy)carbonyl)(methyl)amino)ethyl)(methyl)carbamoyl)oxy)-1-(chloromethyl)-1,2-dihydro-3H-benzo[e]indol-3-yl)-6-oxohexanoyl)-1-(chloromethyl)-2,3-dihydro-1H-benzo[e]indol-5-yl)oxy)carbonyl)(methyl)amino)ethyl)(methyl)carbamoyl)oxy)methyl)phenoxy)-3,4,5-trihydroxytetrahydro-2H-pyran-2-carboxylic acid (7) as a white solid (14.6 mg, 28%). LCMS: Theoretical: C₁₀₀H₁₂₇Cl₂N₉O₃₄ 2067.79, Observed: m/z 1036.10 [M+2H]²⁺.

### Preparation of Compound 8

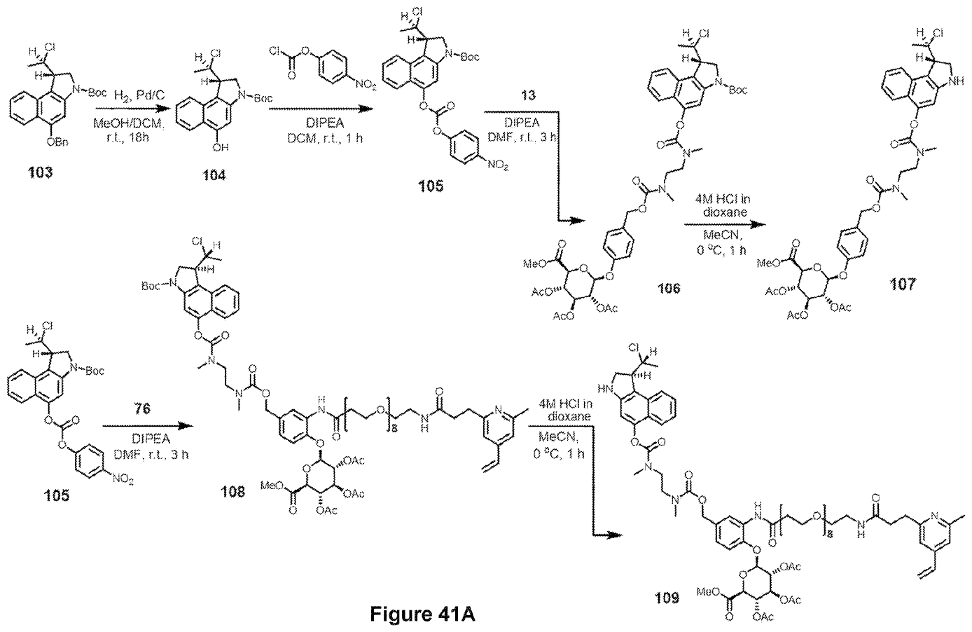

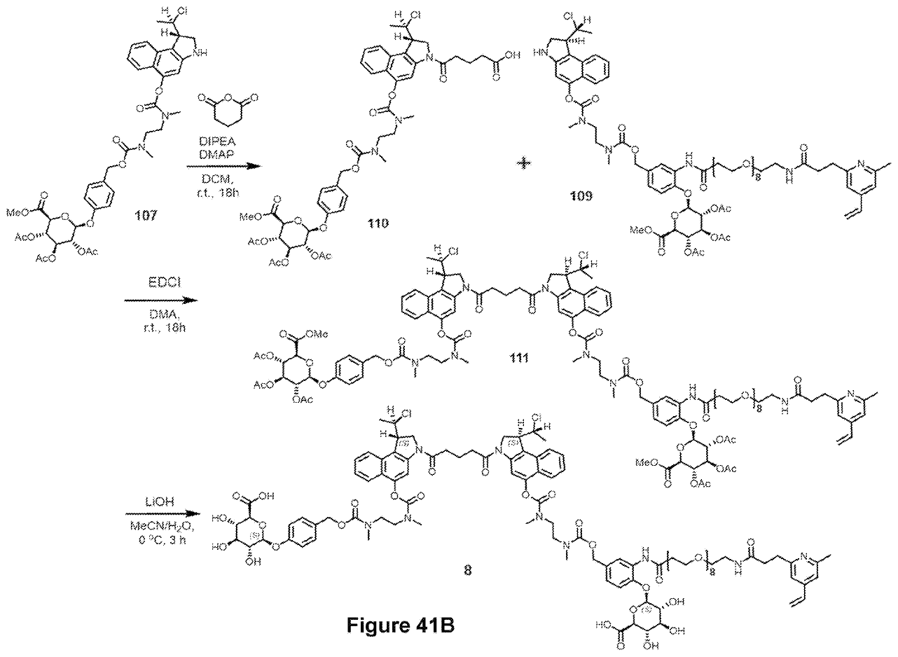

(2S,3S,4S,5R,6S)-6-(4-((((2-(((((S)-3-(5-((S)-5-(((2-((((4-(((2S,3R,4S,5S,6S)-6-carboxy-3,4,5-trihydroxytetrahydro-2H-pyran-2-yl)oxy)-3-(1-(6-methyl-4-vinylpyridin-2-yl)-3-oxo-7,10,13,16,19,22,25,28-octaoxa-4-azahentriacontan-31-amido)benzyl)oxy)carbonyl)(methyl)amino)ethyl)(methyl)carbamoyl)oxy)-1-((R)-1-chloroethyl)-1,2-dihydro-3H-benzo[e]indol-3-yl)-5-oxopentanoyl)-1-((R)-1-chloroethyl)-2,3-dihydro-1H-benzo[e]indol-5-yl)oxy)carbonyl)(methyl)amino)ethyl)(methyl)carbamoyl)oxy)methyl)phenoxy)-3,4,5-trihydroxytetrahydro-2H-pyran-2-carboxylic acid (8) was prepared according to the schemes shown in FIGS. 41A to 41B.

To a solution of tert-butyl (S)-5-(benzyloxy)-1-((R)-1-chloroethyl)-1,2-dihydro-3H-benzo[e]indole-3-carboxylate (103) (0.5 g, 1.14 mmol) in dichloromethane (10 mL) and methanol (10 mL) was added 10% Pd/C (100 mg). After stirring at room temperature for 20 h under H₂ atmosphere, the mixture was filtered. The filtrate was concentrated under vacuum. The crude product was purified by column chromatography on silica gel (eluent petroleum ether/ethyl acetate=10:1) to afford tert-butyl (S)-1-((R)-1-chloroethyl)-5-hydroxy-1,2-dihydro-3H-benzo[e]indole-3-carboxylate (104) as a white solid (300 mg, 76%). LCMS: Theoretical: C₁₉H₂₂ClNO₃ 347.13, Observed: m/z=348.05 [M+H]⁺. ¹H NMR (400 MHz, DMSO-d₆) δ (ppm): 10.28 (s, 1H), 8.08-8.06 (m, 1H), 7.78 (d, J=8.4 Hz, 1H), 7.47-7.45 (m, 1H), 7.44-7.42 (m, 1H), 7.30-7.26 (m, 1H), 4.76-4.74 (m, 1H), 4.11-4.01 (m, 3H), 1.59-1.49 (m, 12H).

To a solution of 104 (300 mg, 864 μmol) and DIPEA (332 mg, 447 μL, 2.57 mmol) in dichloromethane (5.0 mL) was added 4-nitrophenyl chloroformate (260 mg, 1.29 mmol). The mixture was stirred at room temperature for 1 h. The mixture was poured into water (20 mL) and extracted with dichloromethane (20 mL×3). The combined organic layers were dried over Na₂SO₄ and concentrated under vacuum to afford tert-butyl (S)-1-((R)-1-chloroethyl)-5-(((4-nitrophenoxy)carbonyl)oxy)-1,2-dihydro-3H-benzo[e]indole-3-carboxylate (105) as a light-yellow oil (442 mg, crude), which was used directly without further purification.

To a solution of 13 (714 mg, 1.29 mmol) and DIPEA (331 mg, 446 μL, 2.56 mmol) in DMF (5 mL) was added a solution of 105 (442 mg, 864 μmol) in DMF (2 mL). The mixture was stirred at room temperature for 3 h. The mixture was diluted with ethyl acetate (50 mL), washed with water (50 mL) and brine (50 mL), dried over Na₂SO₄, filtered and concentrated. The residue was purified by column chromatography (eluent: petroleum ether/ethyl acetate=1:1) and then by reverse-phase column chromatography (eluent acetonitrile/water=7:3) to afford (2S,3R,4S,5S,6S)-2-(4-((((2-(((((S)-3-(tert-butoxycarbonyl)-1-((R)-1-chloroethyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (106) as light-yellow oil (200 mg, 25%). LCMS: Theoretical: C₄₅H₅₄ClN₃O₁₆ 927.32, Observed: m/z=945.50 [M+NH4]⁺. ¹H NMR (400 MHz, DMSO-d₆) δ 7.96-7.94 (m, 1H), 7.85-7.73 (m, 2H), 7.53 (t, J=7.8 Hz, 1H), 7.38-7.27 (m, 3H), 6.95-6.84 (m, 2H), 5.62-5.57 (m, 1H), 5.45 (s, 1H), 5.10-4.98 (m, 4H), 4.84-4.82 (m, 1H), 4.69-4.65 (m, 1H), 4.18-4.15 (m, 2H), 3.73-3.49 (m, 7H), 3.19-2.86 (m, 6H), 2.01-1.97 (m, 12H), 1.64-1.61 (m, 2H), 1.52-1.50 (m, 9H).

To a solution of 106 (600 mg, 647 μmol) in acetonitrile (10 mL) at 0° C was added 4 N hydrochloric acid/dioxane (10 mL). The mixture was stirred at 0° C for 1 h. The mixture was concentrated under vacuum and triturated with diethyl ether to afford (2S,3R,4S,5S,6S)-2-(4-((((2-(((((S)-1-((R)-1-chloroethyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (107) as a red solid (300 mg, crude), which was used directly without further purification. LCMS: Theoretical: C₄₀H₄₆ClN₃O₁₄ 827.27, Observed: m/z=828.40 [M+H]⁺.

To a solution of 76 (1.0 g, 864 μmol) and DIPEA (222 mg, 299 μL, 1.72 mmol) in DMF (5 mL) was added a solution of 105 (292 mg, 576 μmol) in DMF (2 mL). The mixture was stirred at room temperature for 3 h. The mixture was diluted with EtOAc (100 mL), washed with water (50 mL) and brine (50 mL), dried over Na₂SO₄, filtered and concentrated under vacuum. The residue was purified by column chromatography on silica gel (eluent: petroleum ether/EtOAc=1:1) to afford (2S,3R,4S,5S,6S)-2-(4-((((2-(((((S)-3-(tert-butoxycarbonyl)-1-((R)-1-chloroethyl)-2,3-dihydro-1H-benzo[e]indol-5-yl)oxy)carbonyl)(methyl)amino)ethyl)(methyl)carbamoyl)oxy)methyl)-2-(1-(6-methyl-4-vinylpyridin-2-yl)-3-oxo-7,10,13,16,19,22,25,28-octaoxa-4-azahentriacontan-31-amido)phenoxy)-6-(methoxycarbonyl)tetrahydro-2H-pyran-3,4,5-triyl triacetate (108) as light-yellow oil (288 mg, 33%). LCMS: Theoretical: C₇₅H₁₀₃ClN₆O₂₆ 1538.66, Observed: m/z 779.60 [M+H+Na]²⁺. ¹H NMR (400 MHz, DMSO-d₆) δ (ppm): 8.70-8.65 (m, 1H), 7.97-7.73 (m, 5H), 7.55-7.35 (m, 3H), 7.05 (s, 2H), 6.81-6.74 (m, 1H), 5.58-5.46 (m, 2H), 5.20-5.16 (m, 1H), 5.09-5.13 (m, 3H), 4.84-4.80 (m, 1H), 4.70-4.67 (m, 1H), 4.24-4.14 (m, 3H), 3.65-3.32 (m, 44H), 3.20-2.86 (m, 11H), 2.56 (q, J=5.6, 4.6 Hz, 5H), 2.03-1.98 (m, 9H), 1.66-1.48 (m, 12H).

To a solution of 108 (300 mg, 194 μmol) in acetonitrile (10 mL) at 0° C was added 4 N hydrochloric acid/dioxane (10 mL). The mixture was stirred at 0° C for 1 h. The mixture was concentrated under vacuum and triturated with diethyl ether to afford (2S,3R,4S,5S,6S)-2-(4-((((2-(((((S)-1-((R)-1-chloroethyl)-2,3-dihydro-1H-benzo[e]indol-5-yl)oxy)carbonyl)(methyl)amino)ethyl)(methyl)carbamoyl)oxy)methyl)-2-(1-(6-methyl-4-vinylpyridin-2-yl)-3-oxo-7,10,13,16,19,22,25,28-octaoxa-4-azahentriacontan-31-amido)phenoxy)-6-(methoxycarbonyl)tetrahydro-2H-pyran-3,4,5-triyl triacetate (109) as a red solid (300 mg, crude), which was used directly in next step without further purification. LCMS: Theoretical: C₇₀H₉₅ClN₆O₂₄ 1438.61, Observed: m/z=721.10 [M+2H]²⁺.

To a solution of 107 (500 mg, 604 μmol) and dihydro-2H-pyran-2,6(3H)-dione (374 mg, 3.28 mmol) in dichloromethane (10 mL) was added DIPEA (389 mg, 524 μL, 3.02 mmol) and DMAP (16.8 mg, 138 μmol). The mixture was stirred at room temperature overnight. The mixture was diluted with dichloromethane (100 mL), washed with water (100 mL) and brine (100 mL), dried over Na₂SO₄, filtered and concentrated. The residue was purified by reverse-phase column chromatography (eluent acetonitrile/water (0.1% HCOOH)=9:11) to afford 5-((S)-1-((R)-1-chloroethyl)-5-((methyl(2-(methyl(((4-(((2S,3R,4S,5S,6S)-3,4,5-triacetoxy-6-(methoxycarbonyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoic acid (110) as a light-yellow solid (300 mg, 53%). LCMS: Theoretical: C₄₅H₅₂ClN₃O₁₇ 941.30, Observed: m/z 942.30 [M+H]⁺. ¹H NMR (400 MHz, DMSO-d₆) δ (ppm): 12.16 (s, 1H), 8.20-8.13 (m, 1H), 8.00-7.98 (m, 1H), 7.85-7.80 (m, 1H), 7.56-7.52 (m, 1H), 7.42-7.25 (m, 3H), 7.01-6.84 (m, 2H), 5.65-5.61 (m, 1H), 5.49-5.44 (m, 1H), 5.12-4.99 (m, 4H), 4.86-4.84 (m, 1H), 4.72-4.68 (m, 2H), 4.29 (s, 2H), 3.71-3.63 (m, 5H), 3.48 (s, 2H), 3.19-2.86 (m, 5H), 2.67-2.60 (m, 1H), 2.41-2.23 (m, 2H), 2.01-1.98 (m, 11H), 1.85-1.82 (m, 2H), 1.67 (d, J=6.6 Hz, 3H).

To a solution of 110 (70 mg, 74.3 μmol) and 109 (160 mg, 111 μmol) in DMA (2 mL) was added EDCl (70.9 mg, 457 μmol). The mixture was stirred at room temperature overnight. The mixture was diluted with EtOAc (100 mL), washed with water (50 mL) and brine (50 mL), dried over Na₂SO₄, filtered and concentrated. The residue was purified by prep-TLC (dichloromethane/methanol=20:1) to afford (2S,3R,4S,5S,6S)-2-(4-((((2-(((((S)-1-((R)-1-chloroethyl)-3-(5-((S)-1-((R)-1-chloroethyl)-5-((methyl(2-(methyl(((3-(1-(6-methyl-4-vinylpyridin-2-yl)-3-oxo-7,10,13,16,19,22,25,28-octaoxa-4-azahentriacontan-31-amido)-4-(((2S,3R,4S,5S,6S)-3,4,5-triacetoxy-6-(methoxycarbonyl)tetrahydro-2H-pyran-2-yl)oxy)benzyl)oxy)carbonyl)amino)ethyl)carbamoyl)oxy)-1,2-dihydro-3H-benzo[e]indol-3-yl)-5-oxopentanoyl)-2,3-dihydro-1H-benzo[e]indol-5-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (111) as a yellow solid (25 mg, 14%). LCMS: Theoretical: C₁₁₅H₁₄₅Cl₂NO₄₀ 2361.90, Observed: m/z 1183.15 [M+2H]²⁺.

To a solution of 111 (40 mg, 16.9 μmol) in acetonitrile (1 mL) at 0° C was added a solution of LiOH·H₂O (12.1 mg, 288 μmol) in water (1 mL). The mixture was stirred at 0° C for 3 h. The mixture was acidified with 3 N hydrochloric acid to pH 2-3 and purified by prep-HPLC (eluent 5-80% acetonitrile in water containing 0.1% HCOOH) to obtain (2S,3S,4S,5R,6S)-6-(4-((((2-(((((S)-3-(5-((S)-5-(((2-((((4-(((2S,3R,4S,5S,6S)-6-carboxy-3,4,5-trihydroxytetrahydro-2H-pyran-2-yl)oxy)-3-(1-(6-methyl-4-vinylpyridin-2-yl)-3-oxo-7,10,13,16,19,22,25,28-octaoxa-4-azahentriacontan-31-amido)benzyl)oxy)carbonyl)(methyl)amino)ethyl)(methyl)carbamoyl)oxy)-1-((R)-1-chloroethyl)-1,2-dihydro-3H-benzo[e]indol-3-yl)-5-oxopentanoyl)-1-((R)-1-chloroethyl)-2,3-dihydro-1H-benzo[e]indol-5-yl)oxy)carbonyl)(methyl)amino)ethyl)(methyl)carbamoyl)oxy)methyl)phenoxy)-3,4,5-trihydroxytetrahydro-2H-pyran-2-carboxylic acid (8) (17.0 mg, 49%) as a white solid. LCMS: Theoretical: C₁₀₁H₁₂₉Cl₂N₉O₃₄ 2081.80, Observed: m/z 1042.45 [M+2H]²⁺. ¹H NMR (400 MHz, DMSO-d₆) δ (ppm): 9.11 (s, 1H), 8.23-8.20 (m, 3H), 7.99-7.74 (m, 5H), 7.54 (t, J=7.8 Hz, 2H), 7.46-7.21 (m, 4H), 7.11-6.85 (m, 6H), 6.65 (dd, J=17.6, 10.8 Hz, 1H), 6.05 (d, J=17.8 Hz, 1H), 5.84 (s, 1H), 5.47-5.20 (m, 4H), 5.06-4.77 (m, 8H), 4.33-4.28 (m, 6H), 3.91-3.45 (m, 45H), 3.41-3.37 (m, 6H), 3.20-3.11 (m, 6H), 2.98-2.93 (m, 5H), 2.90-2.86 (m, 5H), 2.71-2.56 (m, 6H), 2.49-2.47 (m, 2H), 2.41 (s, 3H), 2.03-1.97 (m, 2H), 1.67 (d, J=6.6 Hz, 6H).

### Preparation of Antibody-Drug Conjugates (ADCs)

General Procedure for Conjugation of Antibody with Linker-Payload

Protocol A: Isumab01 (an anti-Folate receptor alpha antibody) was partially reduced with 2.2 to 3 molar equivalents of TCEP and conjugated to compounds to 4 in a 8- to 10-fold molar excess and in the presence of 20% (v/v) dimethylacetamide (DMA), or 28-29% (v/v) propylene glycol+1-2% (v/v) DMA, or 26 mM dodecyltrimethylammonium bromide (DTAB) at pH 7.4 for ≥18 h at 25 °C. The resulting ADCs were isolated using Sephadex G25. The ADCs summarised in Table 1 below were generated using Protocol A.

| | | | | DAR |
|---|---|---|---|---|
| ADC | Antibody | Linker-drug | Co-solvent | PLRP |
| Isumab01- | anti-FRα | Compound 1 | 20% (v/v) | 1.7 |
| C1a | | | DMA | |
| Isumab01- | anti-FRα | Compound 1 | 26 mM DTAB | 2.1 |
| C1b | | | | |
| Isumab01- | anti-FRα | Compound 2 | 26 mM DTAB | 1.8 |
| C2a | | | | |
| Isumab01- | anti-FRα | Compound 2 | 28% (v/v) | 2.1 |
| C2b | | | propylene | |
| | | | glycol and 2% | |
| | | | (v/v) DMA | |
| Isumab01- | anti-FRα | Compound 3 | 29% (v/v) | 2.1 |
| C3 | | | propylene | |
| | | | glycol and 1% | |
| | | | (v/v) DMA | |
| Isumab01- | anti-FRα | Compound 4 | 26 mM DTAB | 2.6 |
| C4 | | | | |

Protocol B: Isumab01 (anti-Folate receptor alpha antibody), or Isumab04 (anti-Can Ag antibody), was partially reduced with 2.2 molar equivalents of TCEP and conjugated to compounds 5 to 8 in a 4- or 6-fold molar excess in the presence 2% (v/v) dimethylacetamide (DMA), or without organic solvent, at pH 7.4 for 218 h at 25 °C. The resulting ADCs were isolated using Sephadex G25 or Superdex 200 pg chromatography.

The ADCs summarised in Tables 2 were generated using Protocol B.

| | | | | DAR |
|---|---|---|---|---|
| ADC | Antibody | Linker-drug | Co-solvent | PLRP |
| Isumab01-C5 | anti-FRα | Compound 5 | N/A | 2.3 |
| Isumab04-C5 | anti-CanAg | Compound 5 | N/A | 3.7 |
| Isumab01-C6 | anti-FRα | Compound 6 | 2% (v/v) DMA | 3.3 |
| Isumab04-C6 | anti-CanAg | Compound 6 | 2% (v/v) DMA | 3.7 |
| Isumab01-C7 | anti-FRα | Compound 7 | 2% (v/v) DMA | 3.5 |
| Isumab01-C8 | anti-FRα | Compound 8 | 2% (v/v) DMA | 3.0 |

### Determination of Drug Antibody Ratio (DAR) for ADCs

Determination of average drug-load and drug-load distribution is a crucial attribute as it effects the potency and pharmacokinetics of the ADC. Polymer-Linked Reverse-Phase (PLRP) chromatography is often applied to characterise average drug-load and drug-load distribution for cysteine-linked ADCs.

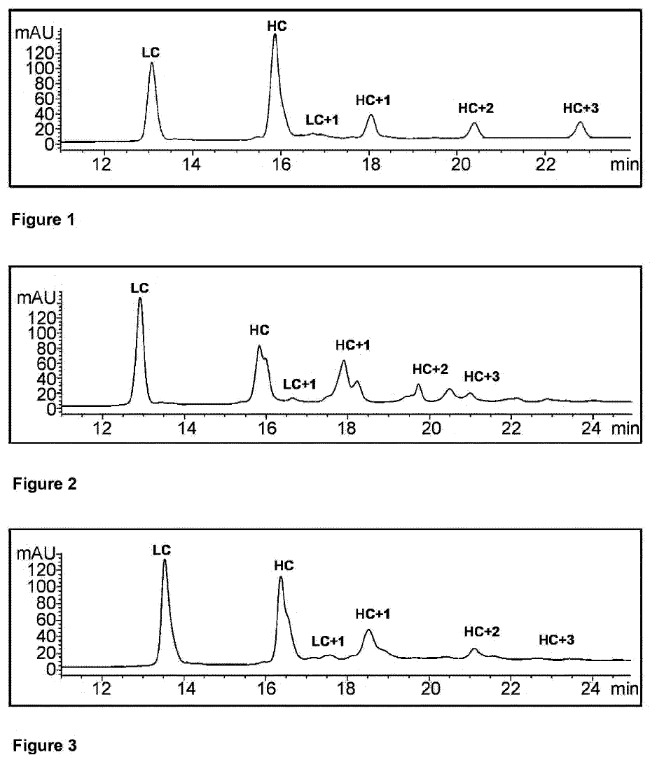

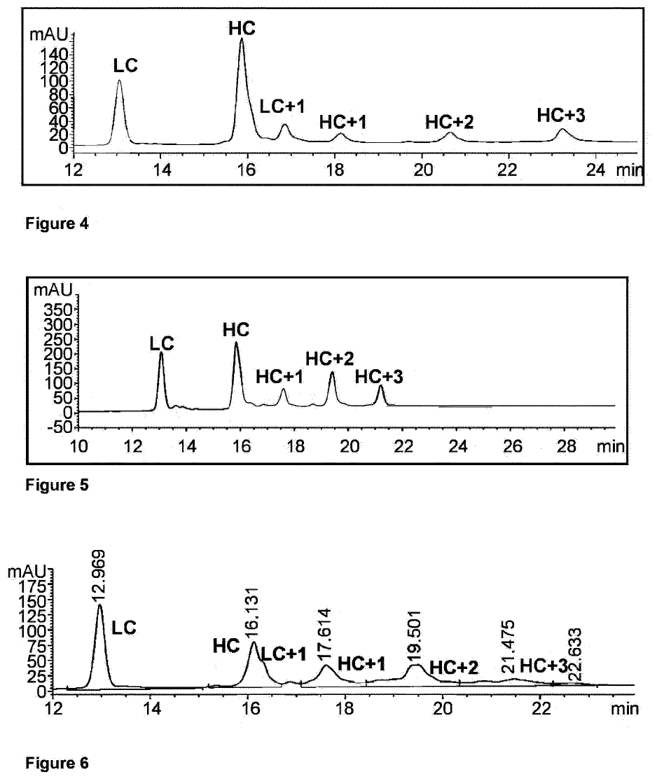

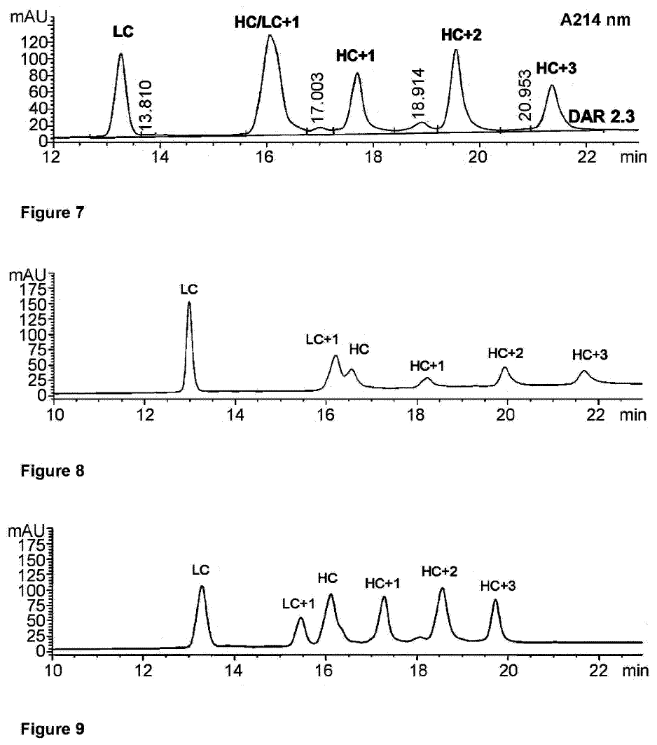

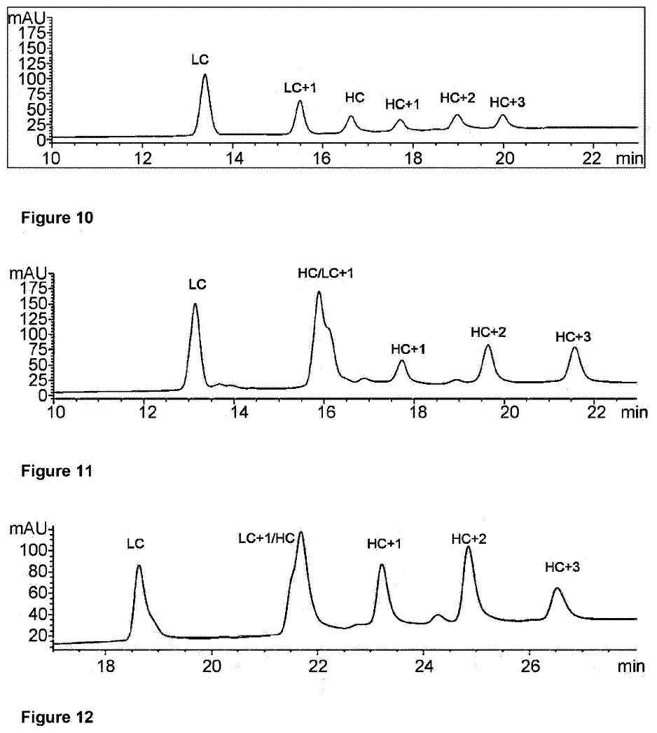

The ADCs of Tables 1 and 2 were subjected to the Polymer-Linked Reverse-Phase (PLRP) analysis. Analysis of the ADCs was accomplished by Polymer-Linked Reverse-Phase (PLRP) chromatography with an Agilent PLRP-S (1000 A, 2.1×50 mm, 5 μm) column. Separation of dithiothreitol (DTT) reduced conjugates via a PLRP column afforded well resolved peaks corresponding to unconjugated or drug conjugated antibody light and heavy chains. The results are shown in FIGS. 1-12.

### Determination of the Aggregation Content of ADCs

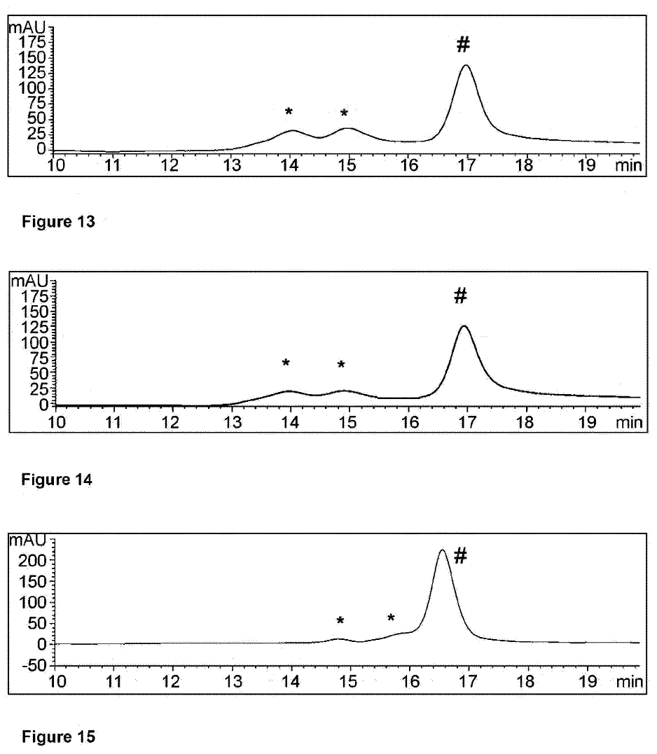

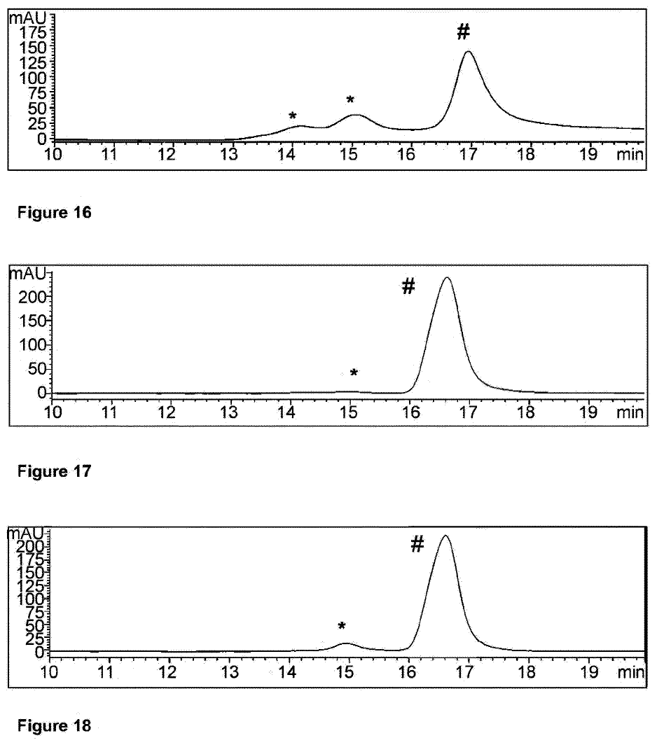

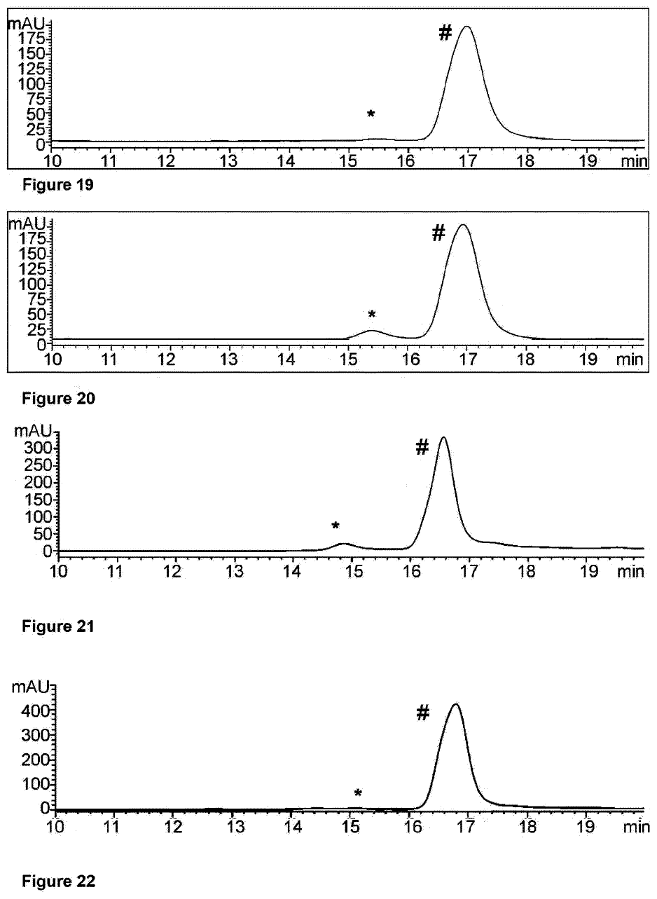

Size Exclusion chromatography (SEC) chromatography is often used to quantify the amount of monomeric and aggregated species in ADCs. Quantification of the aggregated species is important as they can have a significant impact on the pharmacokinetics and biodistribution of an ADC. Separation of aggregated and monomeric ADC species was achieved using a Thermo Scientific MAbPac SEC-1 (5 μm, 7.8×300 nm) column. The SEC analysis and aggregation content of selected ADCs is shown in FIGS. 13-22.

As can be seen, relatively high aggregation levels were observed with ADCs comprising compounds 1 to 4. In contrast, minimal aggregation was observed with ADCs comprising compounds 5 to 8. This indicates that the use of the more hydrophilic glucuronide enzyme cleavable linker instead of a dipeptide linkage, such as valine-citrulline, may reduce linker-drug hydrophobicity and, thus, aggregation of the ADC. Additionally or alternatively, modification of the ethylenediamine-carbamate spacers to include one or more hydrophilic substituents (see, for example, in compound 6) may reduce or further reduce linker-drug hydrophobicity and, thus, aggregation of the ADC.

### Cell Viability Procedure

JEG-3 (placental), OVCAR-3 (ovarian), OV-90 (ovarian), Colo-205 (colon) and NCI-H2110 (lung) cells (all American Type Culture Collection) were obtained by trypsinisation at 37° C/5% CO₂, diluted in an appropriate pre-warmed complete media (CM): JEG-3=EMEM+10% FBS; OVCAR-3=RPMI+20% FBS+0.01 mg/mL bovine insulin; OV-90=1:1 mix of medium 199 and MCDB media+15% FBS; Colo-205 and H2110=RPMI-1640+10% FBS).

After centrifugation (1000 rpm/5 min/4° C), the supernatant was aspirated, and pelleted cells were re-suspended in an appropriate CM. A cell count was taken by haemocytometer and Trypan Blue staining, and an appropriate volume of cells at 5×10⁴ cells/mL was made using the appropriate CM. To white, clear-bottomed microplates (Corning), 100 μL of cells were added to an appropriate number of wells, for a final density of 5000 cells/well, and plates were incubated at 37° C/5% CO₂ for 24 h. To three wells, only appropriate CM was added for subsequent normalisation.

After 24 h, media was removed from all wells and replaced with 100 μL fresh appropriate pre-warmed CM. Compounds were serially diluted in appropriate pre-warmed CM in a 96 well ELISA plate and 20 μL of each dilution was added to appropriate wells. Three wells of adhered cells had no compound added for subsequent normalisation.

For competition assays, the unconjugated antibody of interest was incubated with cells for 30 mins at 37° C/5% CO₂ prior to the addition of the serially diluted ADC. Plates were exposed for 144 h at 37° C/5% before luminescence measurement using Cell Titre Glo reagent (Promega). The reagent was pre-warmed for 30 min at room temperature (RT) before use and was reconstituted as per the manufacturer's instructions. To wells of interest, 100 μL of reagent was added and covered with an adhesive seal. Plates were orbitally shaken for 1 min and left to rest for 10 min within the plate reader before recording luminescence.

Data were normalised to cells alone (100%) and CM alone (0%) using GraphPad Prism. IC₅₀ values were estimated using non-linear regression ([Inhibitor] vs. response—Variable slope (four parameters)).

### Cell Viability Results

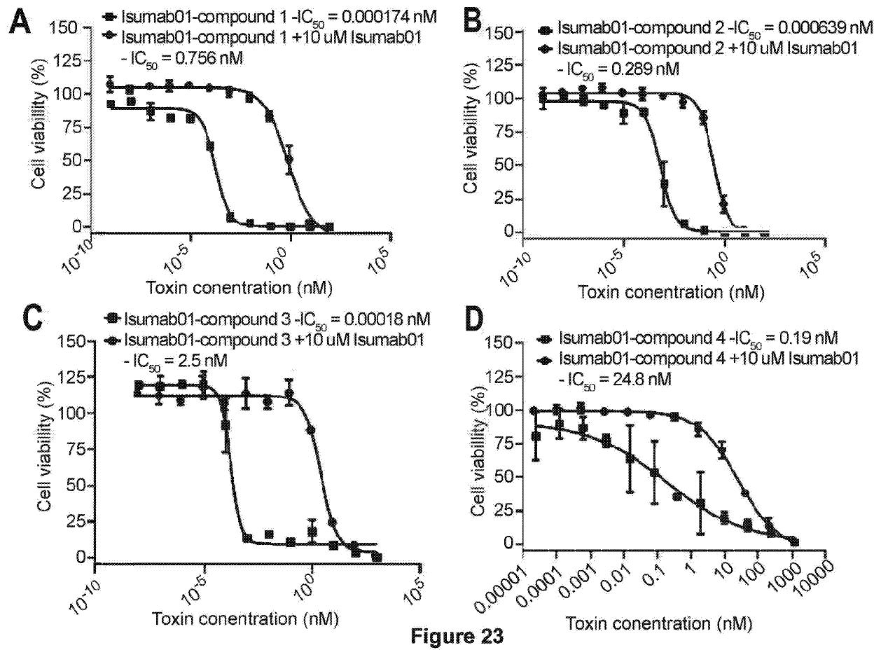

The viability results for selected ADCs of the present invention are shown in FIGS. 23-29. The IC50 data for selected ADCs of the present invention is summarised below in Tables 3 to 7.

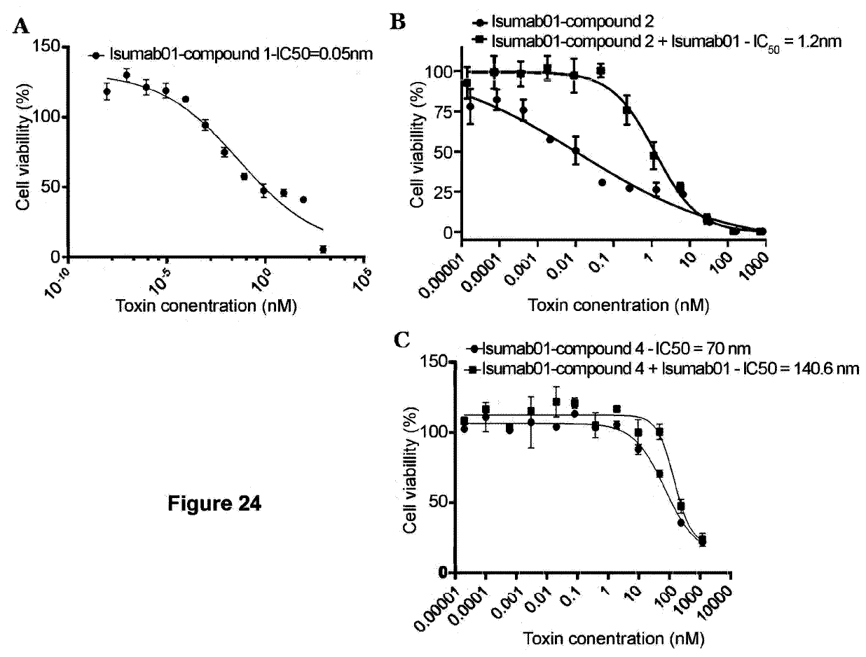

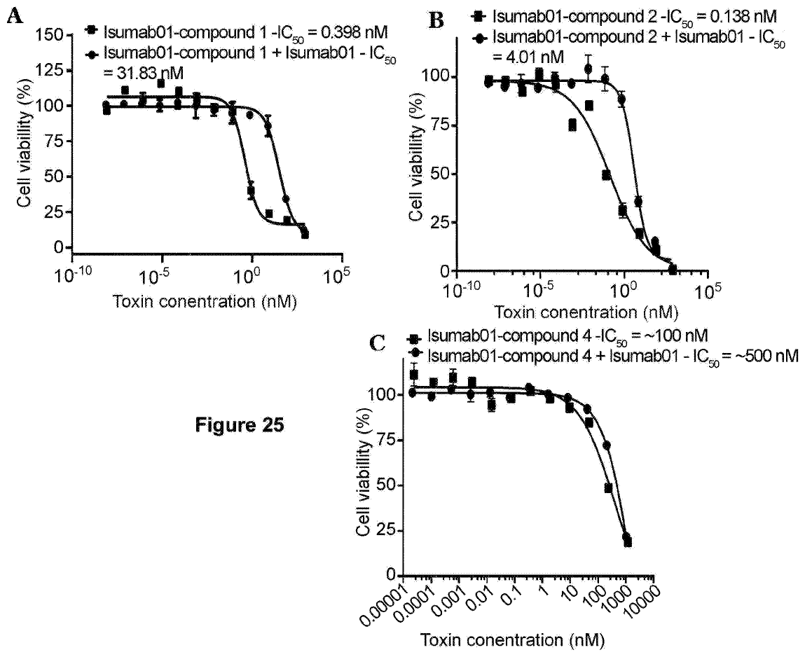

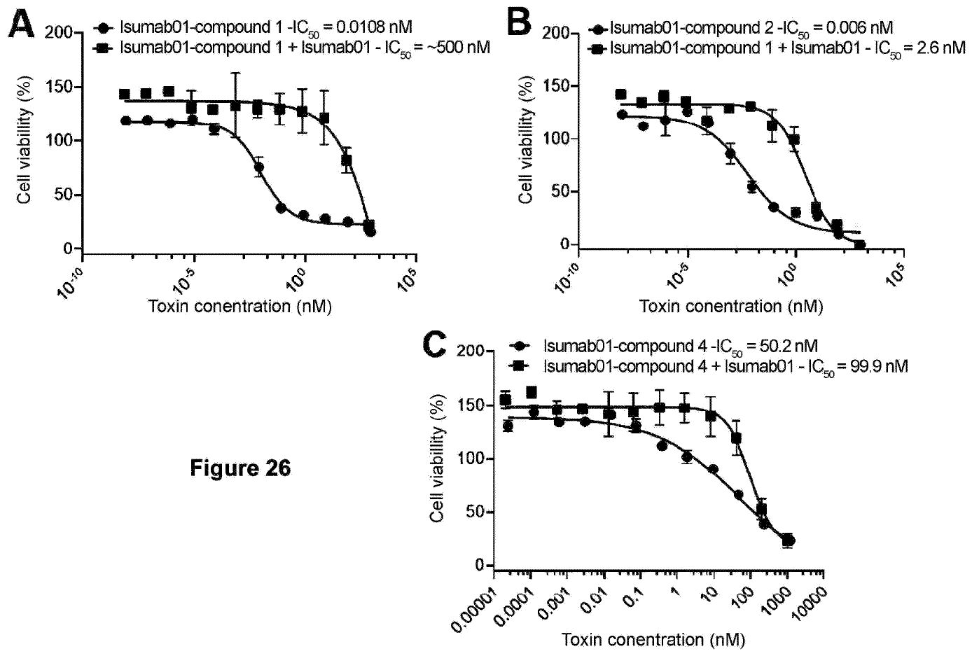

As shown in FIG. 23 and in Table 3, Isumab01-compound 1, Isumab01-compound 2 and Isumab01-compound 3 showed improved in vitro potency in comparison to Isumab01-compound 4 on Jeg-3 cells. This is reflected in the shift from fM IC50 values for Isumab01-compound 1, Isumab01-compound 2 and Isumab01-compound 3 to a pM IC50 value for Isumab01-compound 4. The same trend was maintained for other cancer cell lines. For example, Isumab01-compound 1 and Isumab01-compound 2 showed improved in vitro potency in comparison to Isumab01-compound 4 on Ovcar-3 (See FIG. 24, Table 4), on OV90 cells (See FIG. 25, Table 5) and H2110 cells (See FIG. 26, Table 6). This data highlights the importance of providing a self-immolative spacer between the cleavable sugar prodrug functional group and the first CBI unit.

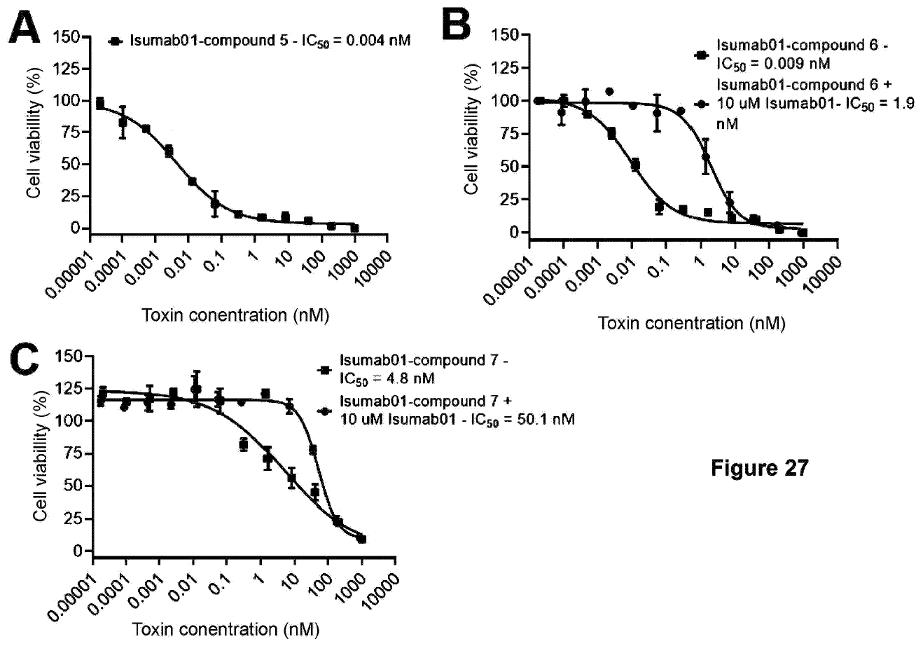

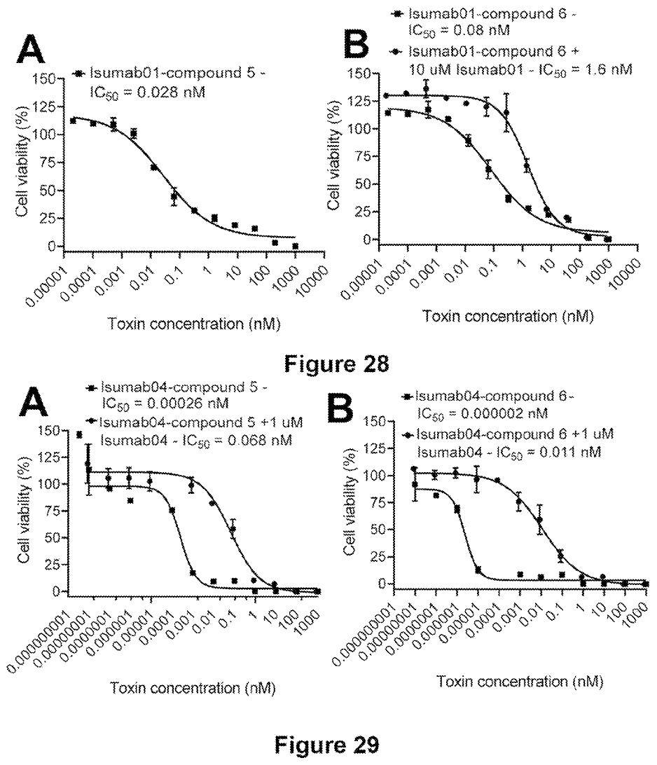

Isumab01-coupled to compound 5 and Isumab01-coupled to compound 6 showed Folate receptor alpha specific potent response on Jeg3 and H2110 cancer cells (see FIG. 27A and B and FIG. 28A and B). The potency of Isumab01-coupled to compound 7 decreased 1200-fold in comparison to Isumab01-coupled to compound 5 on Jeg-3 cells (see FIG. 27C, Table 3). Owing to its lower potency, variants of the linker-drug comprising compound 7 may find utility in ADCs targeting proteins that are overexpressed on cancer cells but also expressed on normal cells, albeit at lower levels. In other words, the lower potency of compound 7 may mitigate the on-target toxicity of ADCs.

Isumab04-coupled to compound 5 and Isumab04-coupled to compound 6 also showed CanAg specific and potent response on Colo205 (see FIG. 29A and B).

TABLE 3
Summary of IC50 for Jeg3 cell viability study

| ADC name | ADC IC50 | ADC + mAb IC50 | |
|---|---|---|---|
| Isumab01-compound 1 | 0.174 pM | 756 | pM |
| Isumab01-compound 2 | 0.639 pM | 289 | pM |
| Isumab01-compound 3 | 0.180 pM | 2500 | pM |
| Isumab01-compound 4 | 190 pM | 25000 | pM |
| Isumab01-compound 5 | 4 pM | N/D* | |
| Isumab01-compound 6 | 9 pM | 1900 | pM |
| Isumab01-compound 7 | 4800 pM | 50 000 | pM |

*N/D-not determined

TABLE 4
Summary of IC50 for OVCAR-3 cell viability study

| ADC name | ADC IC50 | ADC + mAb IC50 |
|---|---|---|
| Isumab01-compound 1 | 50 pM | N/D* |
| Isumab01-compound 2 | 10 pM (Approx) | 1200 pM |
| Isumab01-compound 4 | 70 000 pM | 141000 pM |

TABLE 5
Summary of IC50 for OV90 cell viability study

| ADC name | ADC IC50 | ADC + mAb IC50 |
|---|---|---|
| Isumab01-compound 1 | 398 pM | 32000 pM |
| Isumab01-compound 2 | 138 pM | 4000 pM |
| Isumab01-compound 4 | 100000 pM | 500000 pM |

TABLE 6
Summary of IC50 for H2110 cell viability study

| ADC name | ADC IC50 | ADC + mAb IC50 |
|---|---|---|
| Isumab01-compound 1 | 11 pM | 500000 pM |
| Isumab01-compound 2 | 6 pM | 2600 pM |
| Isumab01-compound 4 | 50000 pM | 100000 pM |
| Isumab01-compound 5 | 28 pM | N/D* |
| Isumab01-compound 6 | 8 pM | 1600 pM |

TABLE 7
Summary of IC50 for Colo205 cell viability study

| ADC name | ADC IC50 | ADC + mAb IC50 |
|---|---|---|
| Isumab04-compound 5 | 0.26 pM | 68 pM |
| Isumab01-compound 6 | 0.002 pM | 11 pM |

### Identification of SLFN11 Marker

The in vitro potency of free payload (compound 112, shown below) was tested across 248 cancer cell lines using Cell Titre Glo reagent (Promega). The compound 112 was synthesised as described in Tietze at al., Angew. Chem. Int. Ed. 2010, 49, 7336-7339.

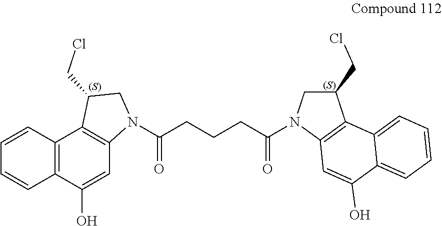

Cells are seeded in growth media in black 384-well tissue culture-treated plates at 500-1500 cells per well. Cells are placed at 37° C 5% CO2 for twenty-four hours before treatment. After 24 h, serially diluted compound 112 was added to appropriate wells. Assay plates are incubated with the compound for 3 days and are then analysed using CellTiter-Glo 2.0. All data points are collected via automated processes and are subject to quality control and analysed using Horizon's proprietary software.

Cell lines were ranked by drug sensitivity using GI50 and a single threshold (median) was determined to define sensitive (responder) and resistant (non-responder) cell lines. Associations of response with mutations, mRNA expression, copy number and tissue type were determined using Horizon's differential analysis software tool and genomic data from the Cancer Cell Line Encyclopedia (CCLE).

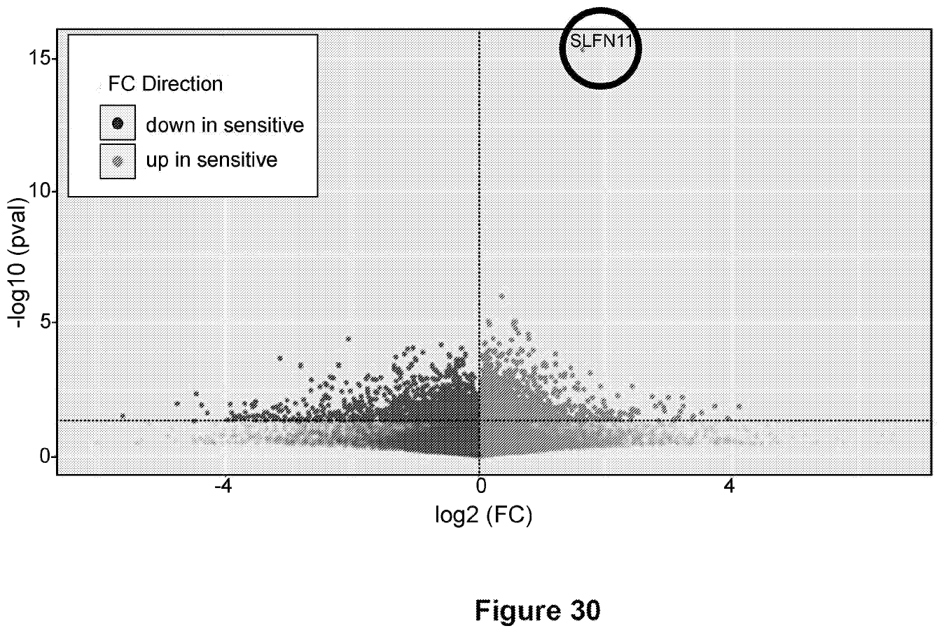

Associations with pathways were assessed by comparing differentially expressed genes to the Molecular Signature Database (MSigDB) and GO Biological Processes pathway sets using Horizon's Alchemy bioinformatics software. It was observed that SLFN11 was the most differentially expressed gene (p-value 2.96×10-16, FDR 1.09×10-11). As shown in FIG. 30, SLFN11 was up-regulated in responders.

### In Vitro Stability Study in IgG Deprived Plasma

1 mg/ml of Adcetris, Enhertu, Isumab01 coupled to compound 5 and Isumab01 coupled to compound 6 were incubated in human and mouse Ig-G depleted plasma.

U.S. Food and Drug Administration (FDA) approved ADCs, Adcetris and Enhertu, were used as control ADCs. 150 μl plasma samples were collected at the following time points 0, 24, 48 and 96 and 168 h and incubated with 75 μl streptavidin magnetic beads coated with Biotin anti-IgG Fc (Human) for 2 h at 22° C in thermomixer (1350 rpm). After incubation beads were washed 3× with 500 μl PBS and eluted with 100 μl 2 mM hydrochloric acid which was neutralized with 0.5 M ammonium bicarbonate pH 8.0. Purified ADCs were analysed on HIC chromatography (DIONEX Ultimate 3000 UHPLC). The analysis was performed on the TSKgel Butyl-NPR column (3.5 cm×4.6 mm, 2.5 μm part. Size) at 30 °C. Gradient: from 0% to 100% Buffer B in 10.5 min was performed at a flow rate of 1.35 mL/min. Buffer A was 50 mM sodium phosphate pH 7.0, 1.5 M (NH4)2SO4 and buffer B was 50 mM sodium phosphate pH 7.0, 20% isopropanol. Detection was done at 280 nm.

Plasma stability was calculated as % change in average drug antibody ratio values (DAR) for Adcetris, % change of intact DAR 8 for Enhertu or % change in average HIC eluted species number for Isumab01-compound 5 and Isumab01-compound 6 at different incubation time points in comparison to day 0.

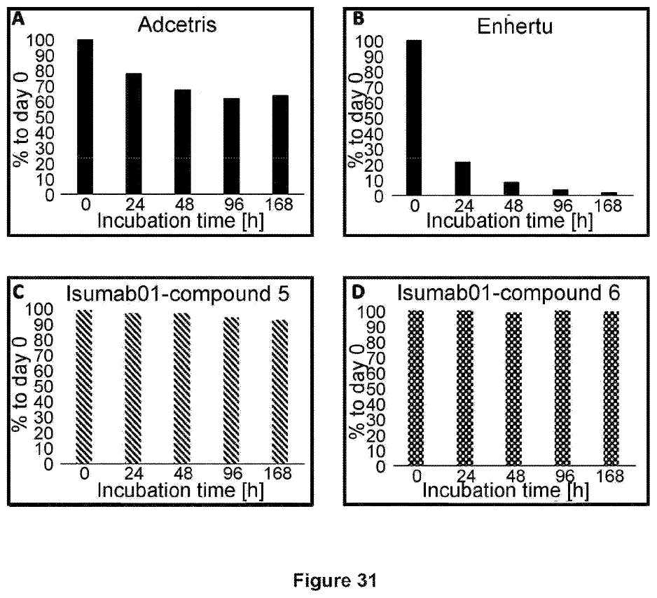

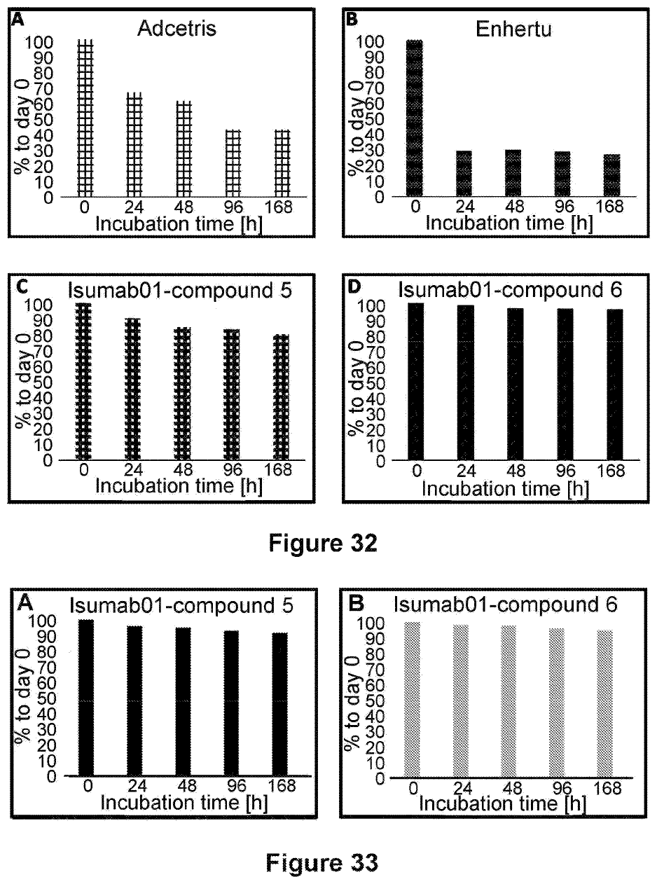

As shown in FIGS. 31C and D and FIGS. 32C and D, Isumab01-compound 5 and Isumab01-compound 6 showed remarkable stability both in human and mouse plasma. Stability in the mouse plasma was unexpected in view of literature reports indicating mouse plasma instability of linkers coupled to compounds containing cyclopropabenzindole (CBI) moieties (see Su et al, Antibody-drug Conjugates Derived From Cytotoxic seco-CBI-Dimer Payloads are Highly Efficacious in Xenograft Models and Form Protein Adducts In Vivo, Bioconjugate Chem. 2019, 30, 5, 1356-1370). In contrast, FIGS. 31A and B and FIGS. 32A and B show that Adcetris and Enhertu exhibit poor stability both in human and mouse plasma.

### In Vitro Stability Study in Whole Mouse Plasma

1 mg/ml of Isumab01 coupled to compound 5 and Isumab01 coupled to compound 6 were incubated in the mouse whole plasma. 100 μl plasma samples were collected at the following time points 0, 24, 48 and 96 and 168 h and incubated with 75 μl streptavidin magnetic beads coated with Biotin anti-IgG Fc (Human) for 2 h at 22° C in thermomixer (1350 rpm). After incubation beads were washed 3× with 500 μl PBS and eluted with 100 μl 2 mM hydrochloric acid which was neutralized with 0.5 M ammonium bicarbonate pH 8.0. Purified ADCs were analysed on HIC chromatography (DIONEX Ultimate 3000 UHPLC). The analysis was performed on the TSKgel Butyl-NPR column (3.5 cm×4.6 mm, 2.5 μm part. Size) at 30 °C. Gradient: from 0% to 100% Buffer B in 10.5 min was performed at a flow rate of 1.35 mL/min. Buffer A was 50 mM sodium phosphate pH 7.0, 1.5 M (NH4)2SO4 and buffer B was 50 mM sodium phosphate pH 7.0, 20% isopropanol. Detection was done at 280 nm.

Plasma stability was calculated as % change in average HIC eluted species number for Isumab01-compound 5 and Isumab01-compound 6. Both Isumab01-compound 5 and Isumab01-compound 6 showed remarkable stability in whole mouse plasma (See FIGS. 33A and B).

### In Vivo Efficacy Studies in Jeg-3 Xenograft Model

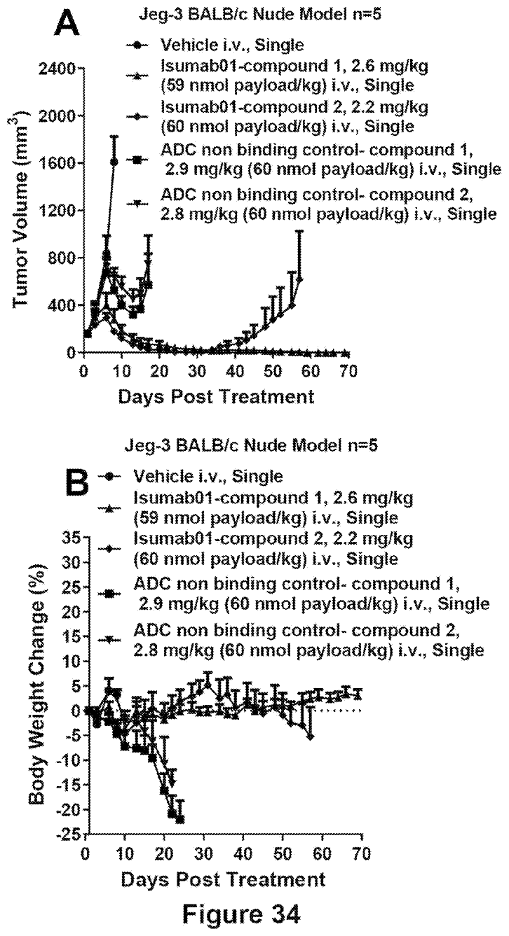

Isumab01-compound 5, Isumab01-compound 6, ADC non-binding control coupled to compound 5 and ADC non-binding control coupled to compound 6 were evaluated in female Balb/c nude mice bearing Jeg-3 xenograft. Mice were subcutaneously inoculated into the right flank with 2×10⁶ Jeg-3 cells in 0.2 mL of DPBS mixed 1:1 with BD Matrigel. Tumour-bearing mice were randomized into groups of 5 animals each and treated with a single intravenous dose of ADC or alternatively with a vehicle solution (PBS) when the average tumour volume reached approximately 160 mm³. Conjugate doses of 2.6 mg/kg (59 nmol payload/kg), 2.2 mg/kg (60 nmol payload/kg), 2.9 mg/kg (60 nmol payload/kg) and 2.8 mg/kg (60 nmol payload/kg) for Isumab01-compound 5, Isumab01-compound 6, ADC non-binding control coupled to compound 5 and ADC non-binding control coupled to compound 6, respectively, were used for the Jeg-3 xenograft study. Tumour size was measured thrice weekly in two dimensions using a calliper, and the volume was expressed in mm³ using the formula: V=0.5 a×b² where a and b are the long and short diameters of the tumour, respectively (see FIG. 34A). Animals were euthanized when tumour volumes reached 2000 mm³. Body weight was also measured thrice weekly as a measure of compound toxicity (see FIG. 34B).

The in vivo effect of ADCs on Jeg-3 tumour xenograft is shown in FIG. 34. Isumab01-compound 5 and Isumab01-compound 6 induced Folate receptor alpha specific complete tumour growth inhibition at 2.6 mg/kg (60 nmol of payload/kg) and 2.2 mg/kg (60 nmol of payload/kg) with no observable toxicity (see FIG. 38B).

Accordingly, ADCs of the present invention possess high therapeutic efficacy and high in-vivo stability.

### Assessment of ADC Tolerability in Cynomolgus Monkey

To assess the safety profile of Isumab04-compound 6, toxicity studies were conducted in naïve cynomolgus monkeys. Anti-CanAg Isumab04 antibody was selected as CanAg is not expressed in cynomolgus monkey normal tissues so any observed adverse effects will be derived from off-target toxicity of ADC. Male monkeys (2 per group) were administered a single dose of 3 mg/kg, 5 mg/kg and 7 mg/kg of Isumab04-compound-6 by intravenous injection.

Clinical signs, body weight, food consumption, clinical pathology and haematology were monitored throughout the study (50 days). On Day 50, all animals were euthanized.

Isumab04-compound-6 was very well tolerated in cynomolgus monkeys up to 7 mg/kg/dose, the highest dose tested. No changes were observed for haematology and clinical chemistry parameters, and body weights were also not affected by the ADC doses. No Isumab04-compound-6-related life-threatening toxicities, irreversible findings, or mortality were observed in the study.

It will be appreciated by persons skilled in the art that the above embodiment has been described by way of example only and not in any limitative sense, and that various alterations and modifications are possible without departing from the scope of the invention as defined by the appended claims.

1. An antibody conjugate or a pharmaceutically acceptable salt or solvate thereof having the formula:
Ab-(L-D)n
wherein:
Ab is an antibody or antigen-binding fragment thereof;
D is a prodrug;
L is a linker covalently connecting Ab to D; n is an integer from 1 to 20;
Wherein:
D is represented by the formula: 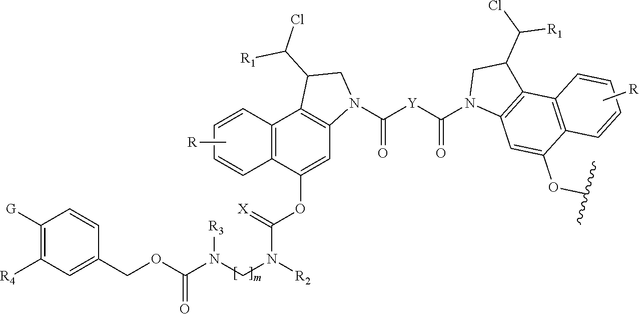
Wherein:
the wavy line indicates the covalent attachment to L;
R is H or an optionally substituted C₁-C₄ alkyl group, an optionally substituted C₁-C₄ alkoxy group, an optionally substituted aryl group, an optionally substituted heteroaryl group, an optionally substituted C₁-C₄ alkyl carboxy C₁-C₄ alkyl group, F, Cl, Br, or I, CN, an optionally substituted C₁-C₄ alkylsulfonyl group, an optionally substituted arylsulfonyl group, or an NR_{z} group, where NR_{z} is selected from H, optionally substituted C₁-C₄ alkyl group or optionally substituted C₁-C₄ acyl; R₁ is H, a C₁-C₄ alkyl group, or a C₁-C₄ alkoxy group;
Y is selected from an optionally substituted C1-C10 alkyl group, or a group having the formula:
^{O}[C(_{RA})₂]—X₃—[C(R_{A})₂]_{P}
where o and p are independently from one another selected from an integer of 1 to 20, whereby o and p may be the same integer or a different integer, X₃ is i) N, S or O, or ii) an aryl group or a heteroaryl group, wherein [C(R_{A})₂]O and [C(R_{A})₂]_{P} are present in the meta position of said aryl group or said heteroaryl group, and each RA is independently from one another selected from H or an optionally substituted C₁-C₄ alkyl group or an optionally substituted C₁-C₄ acyl group;
X is O or S;
m is an integer from 1 to 20;
R₂ and R₃ are independently selected from H, C₁-C₂₀ alkyl, and —C₁-C₈ heteroalkyl;
R₄ is selected from H or an electron-withdrawing group, and
G is a sugar moiety selected from β-D-galactoside, β-D-glucuronide, β-D-glucoside, α-D-mannoside, or fucoside.

2. The antibody conjugate of claim 1, wherein R₂ and R₃ are independently selected from —C₁-C₂₀ alkyl and —(CH₂CH₂O)ᵣH, where r is an integer from 1 to 4.

3. The antibody conjugate of claim 1, wherein L has the formula:
L^{A}-L^{B}-L^{C}-L^{D}
wherein:
L^{A} is connecting group or bond linking Ab to L^{B};
L^{B} is an enzyme cleavable linker;
L^{C} is a self-immolative spacer or absent; and
L^{D} is spacer group covalently bound to D.

4. The antibody conjugate of claim 3, wherein L_{B} is selected from a β-D-glucuronide linker or a β-D-galactoside linker and L_{C} is absent.

5. The antibody conjugate of claim 3, wherein L^{B} is a dipeptide linker selected from -Valine-Citrulline- (-Val-Cit-), -Valine-Alanine- (-Val-Ala-), -Valine-Lysine- (-Val-Lys-), -Valine-Arginine- (-Val-Arg-), -Phenylalanine Acid-Citrulline- (-Phe-Cit-), -Phenylalanine-Lysine- (-Phe-Lys-), or -Phenylalanine-Arginine- (-Phe-Arg-), wherein L^{C} is para-aminobenzyl, para-aminobenzyloxycarbonyl.

6. (canceled)

7. The antibody conjugate of claim 3, wherein L^{D} is selected from —C(O)N(R_{B})(R_{C}) or —C(S)N(R_{B})(R_{C}), wherein L^{D} is bonded to D at the carbon of the C(O) or C(S) group, wherein R_{B} is —C₁-C₂₀ alkylN(R_{D})—, and wherein R_{C} and R_{D} are independently selected from H, C₁-C₂₀ alkyl, and —C₁-C₈ heteroalkyl.

8. (canceled)

9. The antibody conjugate of claim 1, wherein the electron-withdrawing group is a nitro group, and G is β-D-galactoside or β-D-glucuronide.

10. (canceled)

11. The antibody conjugate of claim 1, comprising a structure selected from:

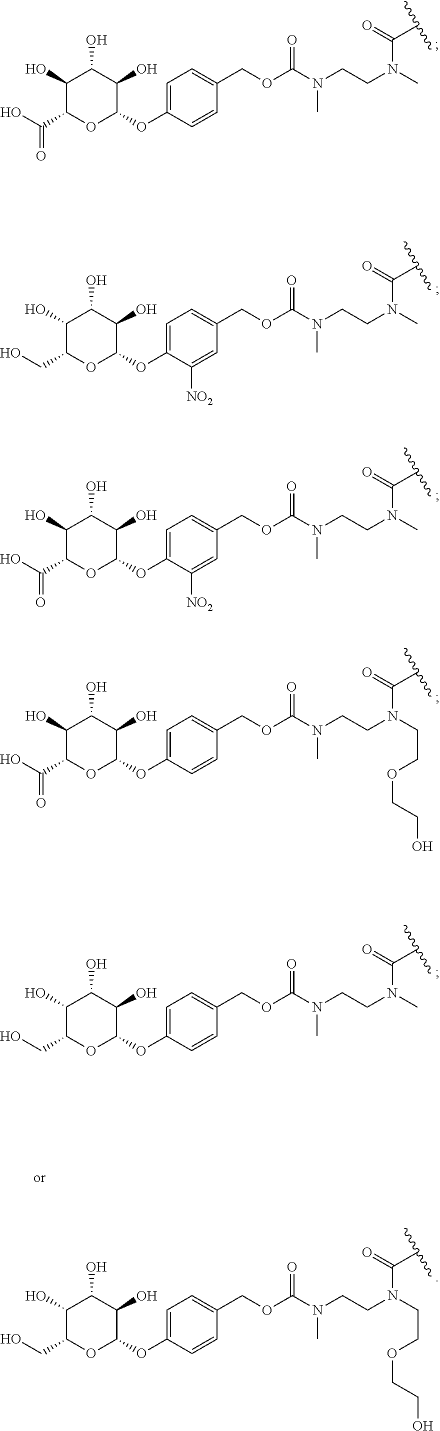

12. The antibody conjugate of claim 1, wherein L^{A} has the formula: 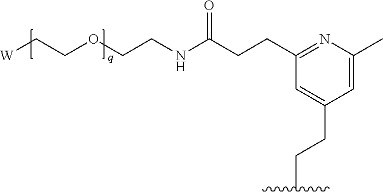
wherein:
the wavy line indicates a point of attachment to Ab;
W is an amide coupling group for connecting L^{A} with L^{B}; and
q is an integer from 1 to 20.

13. The antibody conjugate of claim 1, wherein L is covalently bound to Ab by a thioether bond, and the thioether bond comprises a sulfur atom of a cysteine of the Ab.

14. (canceled)

15. The antibody conjugate of claim 1, wherein the antibody is selected from the group consisting of an anti-folate receptor alpha antibody, an anti-CanAg antibody, anti-B7H3, anti-MSLN, anti-Trop2, anti-5T4, anti-CD20, anti-PSMA, anti-EGFR, anti-CD70, anti-DLL3, an anti-ROR1 antibody, an anti-c-MET antibody, an anti-Her3 antibody, an anti-EphA3 antibody, an anti-CD30 antibody, an anti-CD79 antibody, an anti-NaPi3 antibody an anti-CD22 antibody, an anti-CD19 antibody, an anti-CD33 antibody, an anti-Her2 antibody, and an anti-MUC16.

16. A linker-drug intermediate having the formula:
Lp-D
wherein:
Lₚ is a linker precursor comprising a thiol-reactive functional group; and
Wherein:
D is represented by the formula 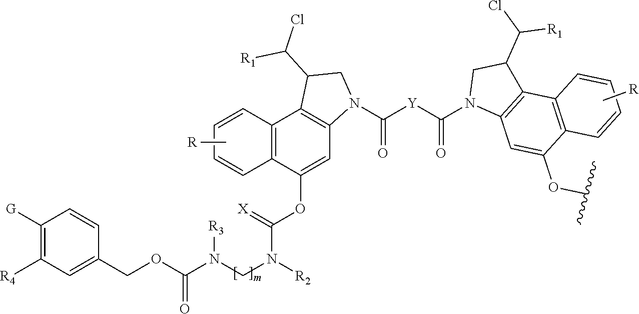
Wherein:
the wavy line indicates the covalent attachment to L;
R is H or an optionally substituted C₁-C₄ alkyl group, an optionally substituted C₁-C₄ alkoxy group, an optionally substituted aryl group, an optionally substituted heteroaryl group, an optionally substituted C₁-C₄ alkyl carboxy C₁-C₄ alkyl group, F, Cl, Br, or I, CN, an optionally substituted C₁-C₄ alkylsulfonyl group, an optionally substituted arylsulfonyl group, or an NR_{z} group, where NR_{z} is selected from H, optionally substituted C₁-C₄ alkyl group or optionally substituted C₁-C₄ acyl;
R₁ is H, a C₁-C₄ alkyl group, or a C₁-C₄ alkoxy group;
Y is selected from an optionally substituted C₁-C₁₀ alkyl group, or a group having the formula:
_{O}[C(R_{A})₂]—X₃—[C(R_{A})₂]_{P}
where o and p are independently from one another selected from an integer of 1 to 20, whereby o and p may be the same integer or a different integer, X₃ is i) N, S or O, or ii) an aryl group or a heteroaryl group, wherein [C(RA)2]O and [C(RA)2]_{P} are present in the meta position of said aryl group or said heteroaryl group, and each RA is independently from one another selected from H or an optionally substituted C₁-C₄ alkyl group or an optionally substituted C₁-C₄ acyl group;
X is O or S; m is an integer from 1 to 20;
R₂ and R₃ are independently selected from H, C₁-C₂₀ alkyl, and —C₁-C₈ heteroalkyl;
R₄ is selected from H or an electron-withdrawing group, and
G is a sugar moiety selected from β-D-galactoside, β-D-glucuronide, β-D-glucoside, α-D-mannoside, or fucose.

17. (canceled)

18. The linker-drug intermediate of claim 16, wherein Lₚ has the formula:
L^{A}-L^{B}-L^{C}-L^{D}
wherein:
L^{A} is a connecting group comprising the thiol-reactive functional group;
L^{B} is an enzyme cleavable linker;
L^{C} is a self-immolative spacer or absent; and
L^{D} is spacer group covalently bound to D.

19. The linker-drug intermediate of claim 18, wherein LB is selected from a p-glucuronide linker or a p-galactoside linker and Lc is absent.

20. The linker-drug intermediate of claim 18, wherein L^{B} is a dipeptide linker selected from -Valine-Citrulline- (-Val-Cit-), -Valine-Alanine- (-Val-Ala-), -Valine-Lysine- (-Val-Lys-), -Valine-Arginine- (-Val-Arg-), -Phenylalanine Acid-Citrulline- (-Phe-Cit-), -Phenylalanine-Lysine- (-Phe-Lys-), or -Phenylalanine-Arginine- (-Phe-Arg-).

21. (canceled)

22. The linker-drug intermediate of claim 16, wherein L^{D} is selected from —C(O)N(R_{B})(R_{C}) or —C(S)N(R_{B})(R_{C}), wherein L^{D} is bonded to D at the carbon of the C(O) or C(S) group, wherein RB is —C₁-C₂₀ alkylN(R_{O})—, and wherein R_{C} and R_{D} are independently selected from H, C₁-C₂₀ alkyl, and —C₁-C₈ heteroalkyl.

23. (canceled)

24. (canceled)

25. (canceled)

26. The linker-drug intermediate of claim 16, wherein L^{A} has the formula: 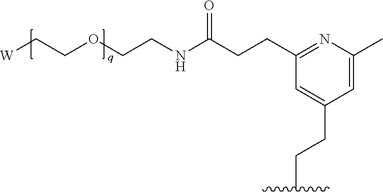
wherein:
W is an amide coupling group for connecting L^{A} with L^{B}; and
q is an integer from 1 to 20.

27. The linker-drug intermediate of claim 26, selected from:

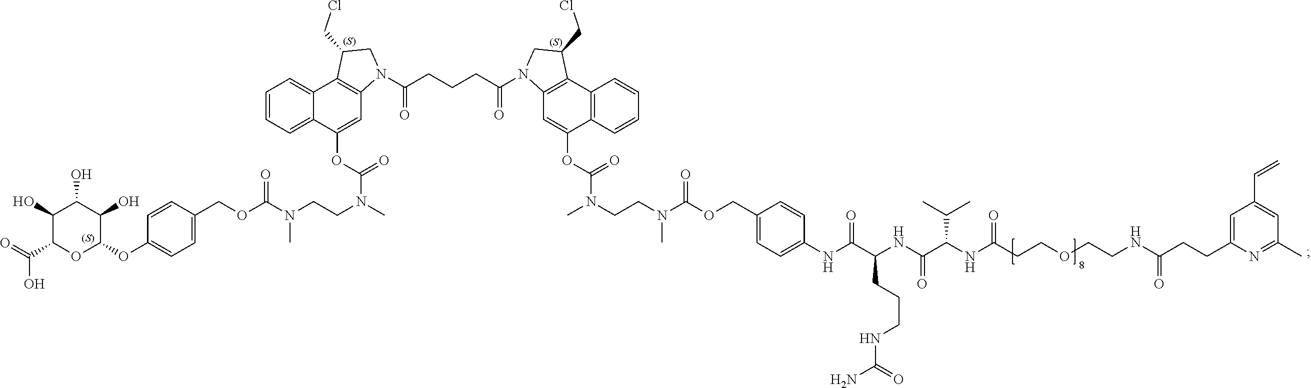

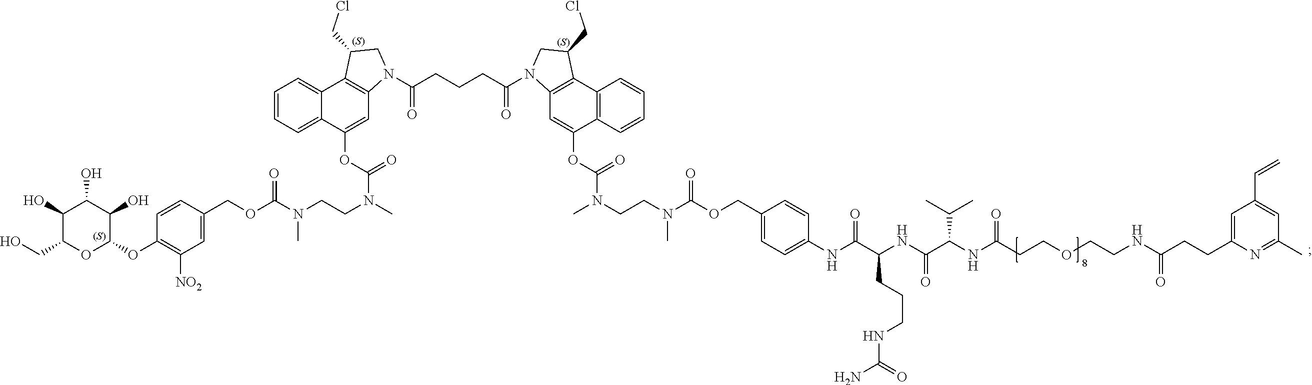

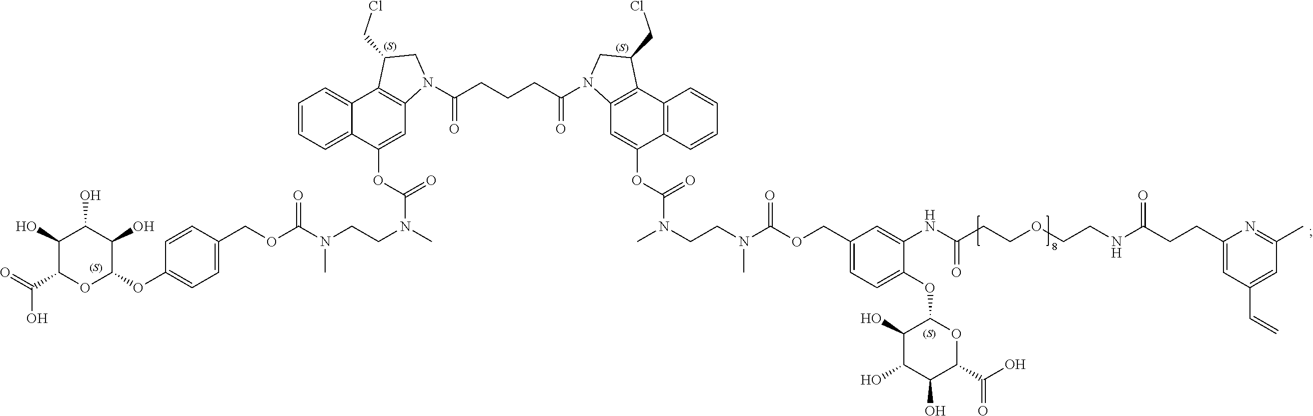

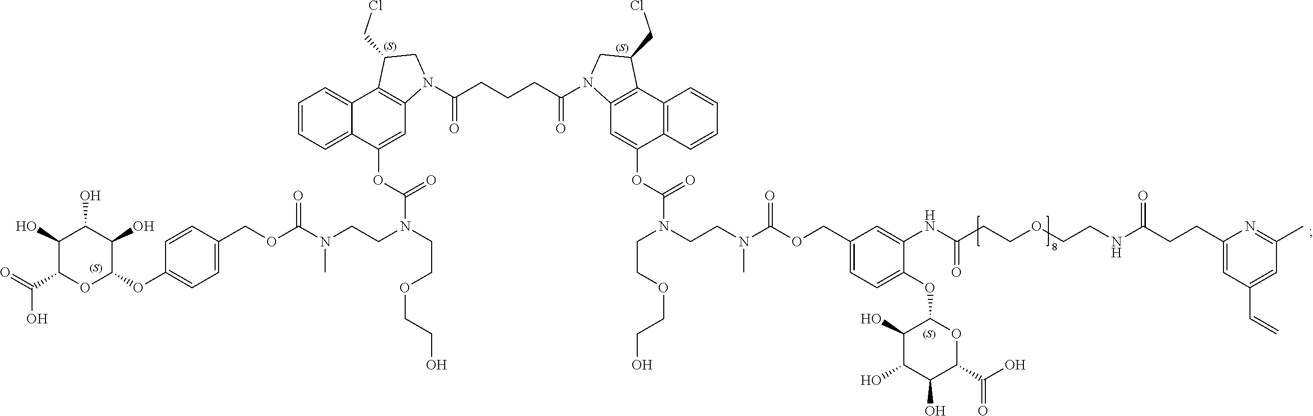

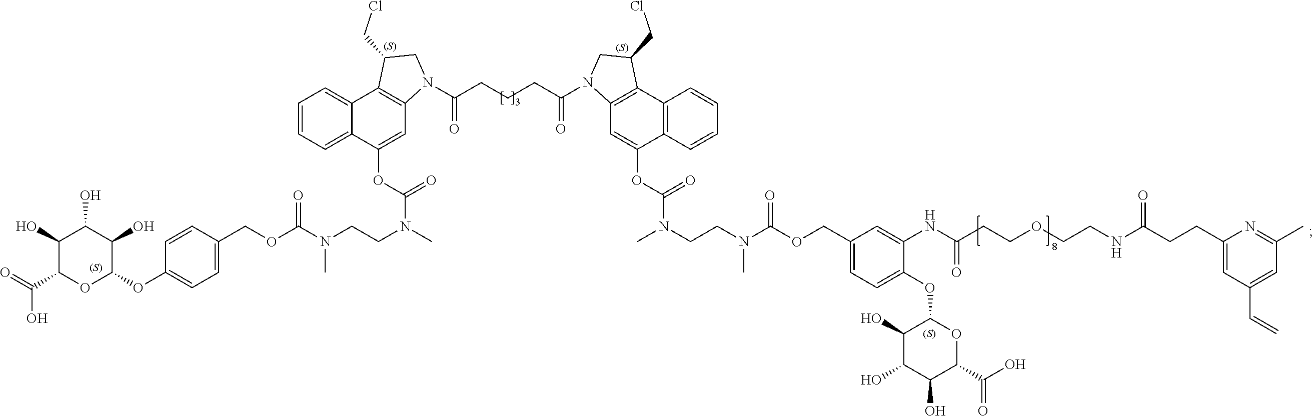

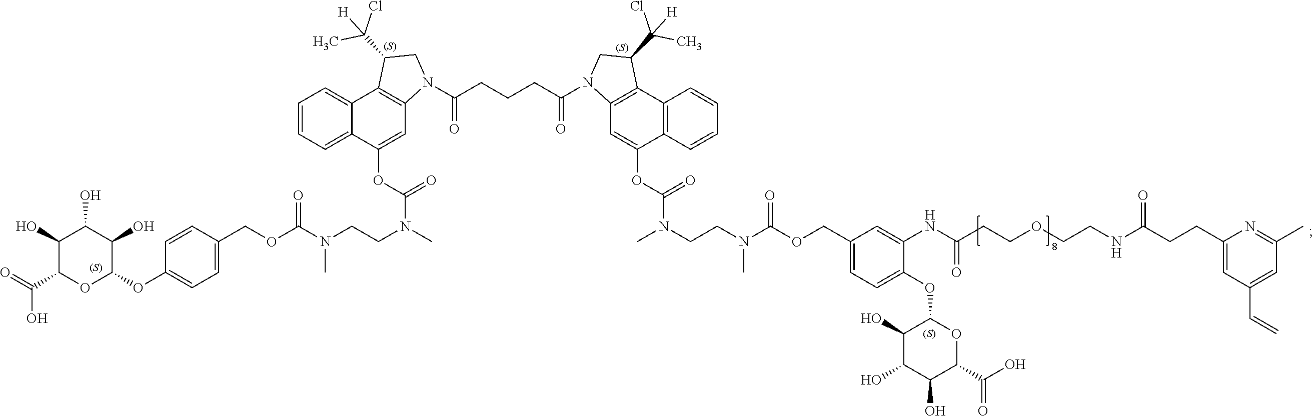

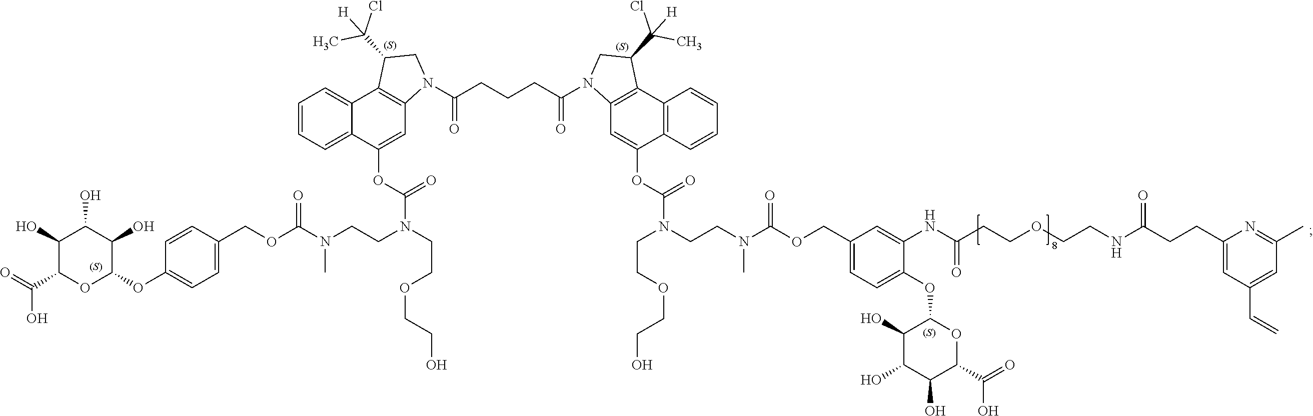

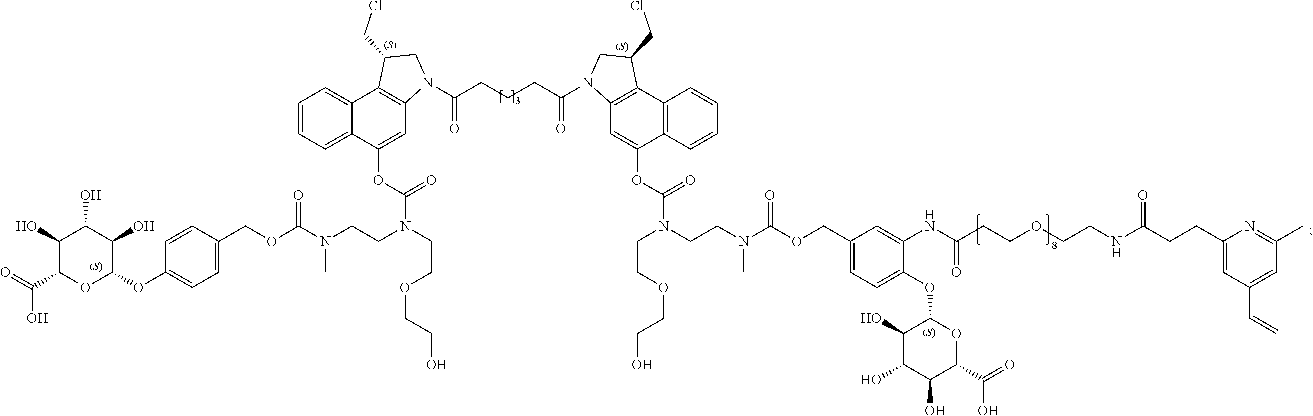

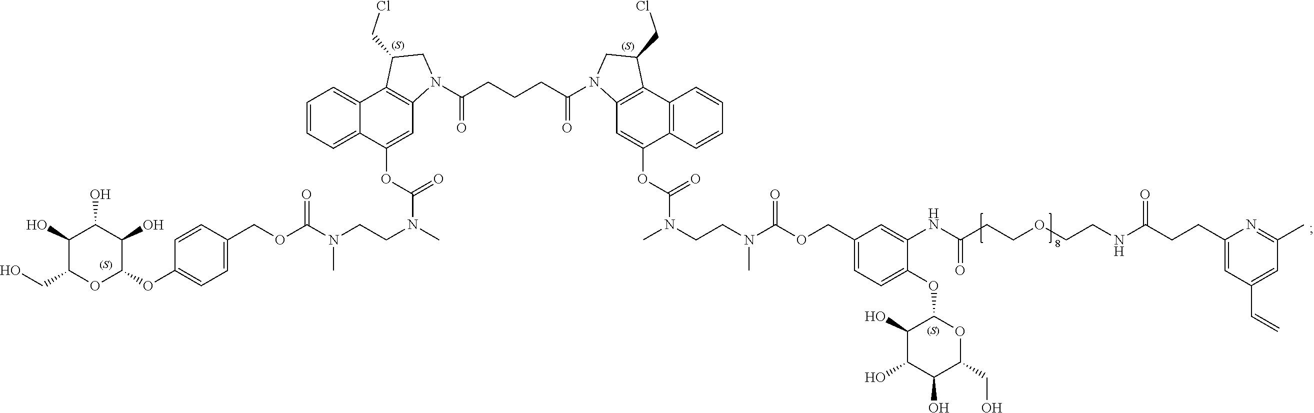

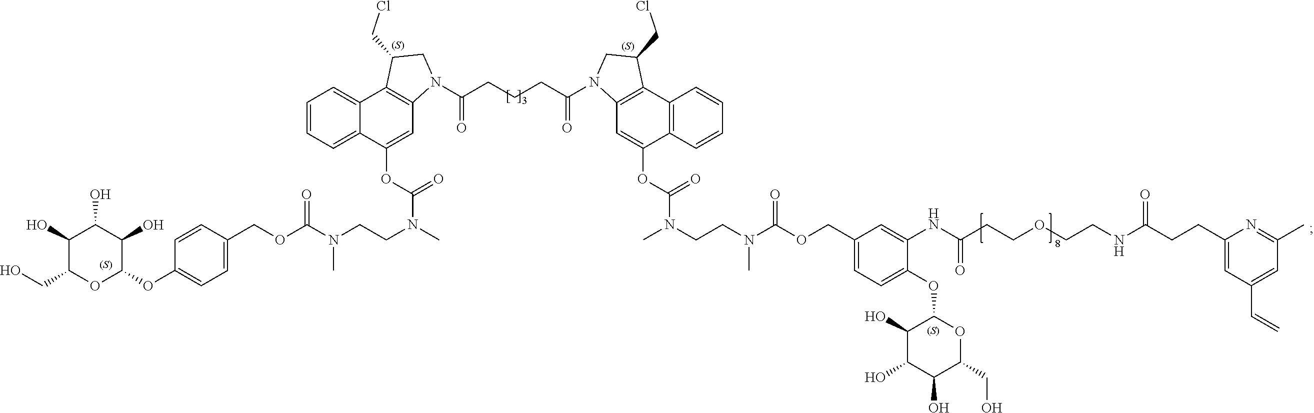

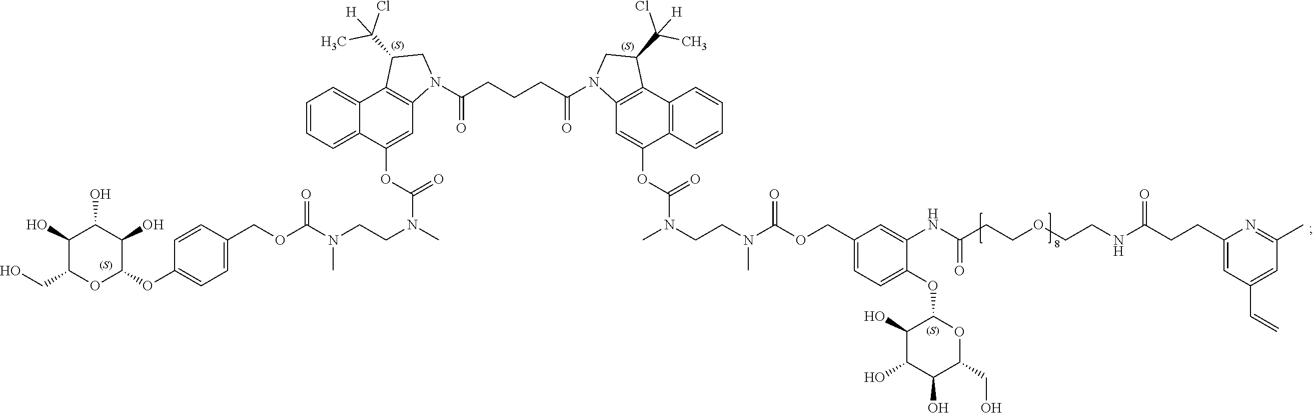

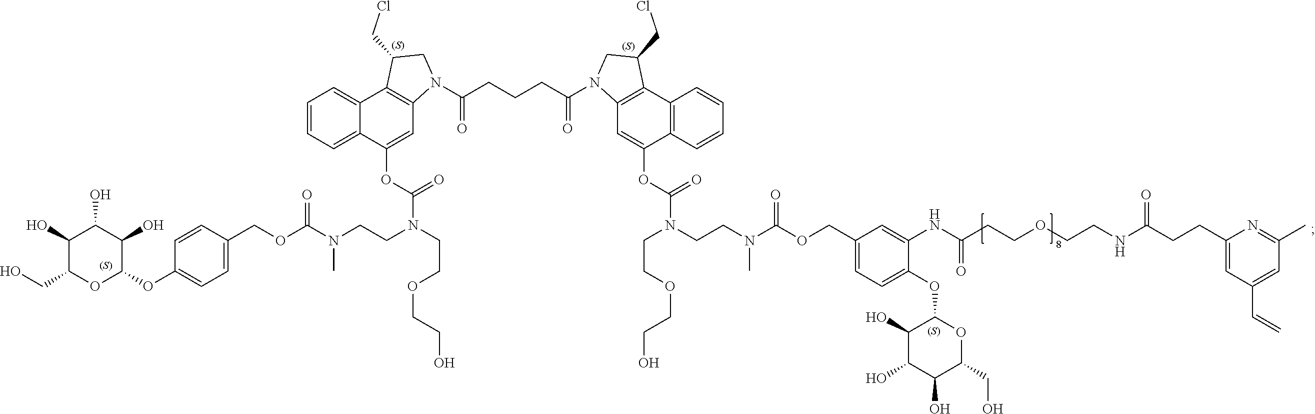

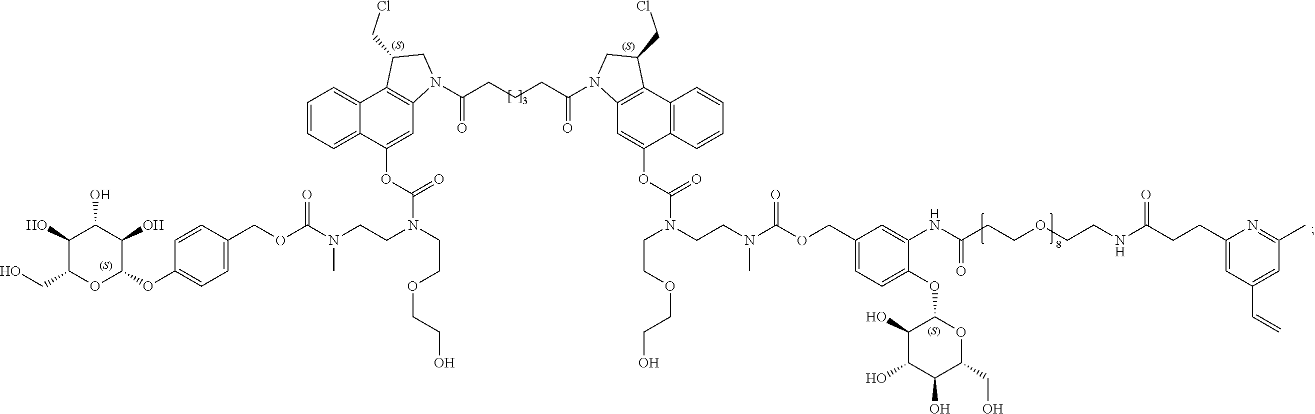

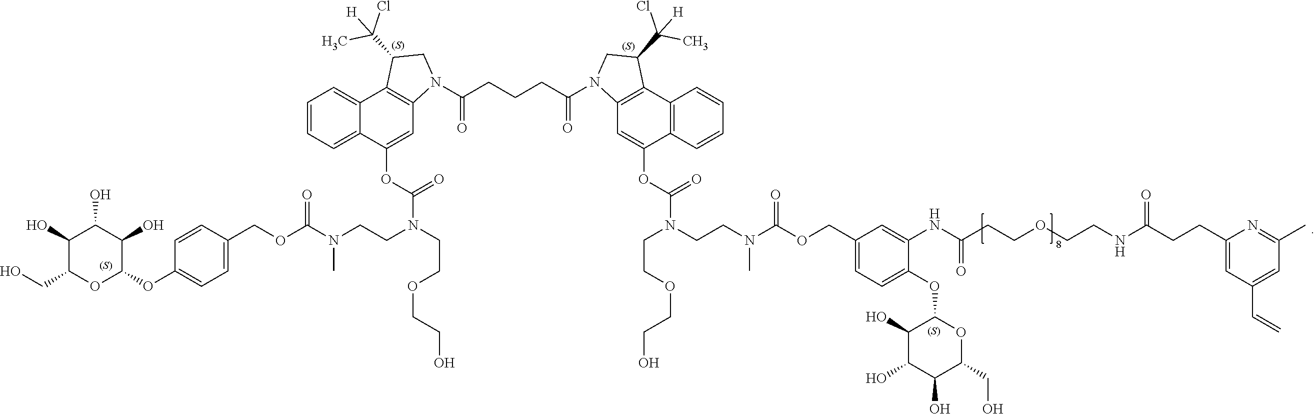

28. A pharmaceutical composition comprising an antibody conjugate according to claim 1; and one or more pharmaceutically acceptable excipients, diluents, or carriers, wherein the pharmaceutical composition is for use as a medicament.

29. (canceled)

30. (canceled)

31. (canceled)

32. A method of producing the antibody conjugate according to claim 1, comprising:
providing a thiol group at one or more desired positions on the antibody or antigen binding fragment thereof (Ab), and
contacting the antibody or antigen binding fragment thereof (Ab) with a linker-drug intermediate.

33. (canceled)
